(19) United States
(12) Patent Application Publication
(10) Pub. No.: US 2025/0275973 A1
(43) Pub. Date: Sep. 4, 2025

(54) USE OF ALC1 INHIBITORS AND SYNERGY WITH PARPI

(71) Applicant: EISBACH BIO GMBH, Martinsried (DE)

(72) Inventors: William M. MENZER, Martinsried (DE); Katharina SAHIRI, Martinsried (DE); Adrian SCHOMBURG, Martinsried (DE); Andreas LADURNER, Martinsried (DE); Peter SENNHENN, Martinsried (DE)

(21) Appl. No.: 18/862,525

(22) PCT Filed: May 2, 2023
(86) PCT No.: PCT/EP2023/061584

§ 371 Date: Nov. 1, 2024

(30) Foreign Application Priority Data
May 2, 2022 (EP) 22171217.7

(51) Int. Cl. A61K 31/517; A61K 31/454; A61K 31/502; A61K 31/5025; A61P 35/00

(52) U.S. Cl. CPC A61K 31/517; A61K 31/454; A61K 31/502; A61K 31/5025; A61P 35/00

(57) ABSTRACT

The present invention relates to the use of small molecule compounds that allosterically inhibit ALC1 (CHD1L) and which induce the trapping of PARP1, PARP2 and/or PARP3 on chromatin or at DNA damage sites for the treatment of proliferative diseases. Disruption of the chromatin remodeling forces of ALC1 through these agents enables a highly selective therapy for targeting the DNA damage functions of PARP enzymes in several proliferative diseases, notably BRCA-deficient cancers. Via inhibition of the enzymatic activity, the compounds engage the synthetic lethality between mutations in HRD pathways, including BRCA1/2 and ALC1. By trapping PARP enzymes, inhibitors of ALC1 potentiate the cancer cell killing properties of PARP inhibitors, enable therapeutic approaches where ALC1 is amplified as an oncogene, therapeutically make it possible to overcome PARP inhibitor resistance mechanisms and enable an alternative approach to the treatment of germline or acquired BRCA1/BRCA2 deficiency, including tumors defined by “BRCAness” or other changes in DNA repair networks.

The present invention relates to small molecule compounds that allosterically inhibit ALC1 (CHD1L) and are synergistic the inhibition of the PARP family of enzymes. These inhibitors possess anti-proliferative activity in cancers that acquired a BRCA1 or BRCA2 deficiency. Disruption of the chromatin remodeling forces of ALC1 through these agents enables a highly selective therapy for targeting the DNA damage functions of PARP enzymes in several proliferative diseases, notably BRCA-deficient cancers. Via inhibition of the enzymatic activity, the compounds engage the synthetic lethality between BRCA1/2 and ALC1. By being synergistic with PARP enzyme inhibitors, ALC1 inhibitors potentiate the cancer cell killing properties of these PARP inhibitors, enabling therapeutic approaches where ALC1 is amplified as an oncogene. Additionally, these ALC1 inhibitors make it possible to overcome PARP inhibitor resistance mechanisms and enable an alternative approach to the treatment of germline or acquired BRCA1/BRCA2 deficiency, including tumors defined by “BRCAness” or other changes in DNA repair networks.

### BACKGROUND OF THE INVENTION

For recognition of single-strand and double-strand breaks (SSBs/DSBs), the nuclear Poly-ADP-ribose polymerase (PARP) enzymes are early key factors in the DNA damage response (DDR). Using the metabolite NAD⁺, PARP-1 and -2 add poly-ADP-ribose (PAR) chains to chromatin components and to factors belonging to the DDR, while PARP-3 targets chromatin components via mono-ADP-ribosylation. PARPs get recruited to DNA lesions by recognizing specifically altered, DNA-damage induced structures, which activates their PARylation activity, which in turn regulates their activity and the activity of other DDR and chromatin proteins, facilitating the DDR (Ray Chaudhuri and Nussenzweig, 2017).

This catalytic activity can be inhibited by NAD⁺ analogues and has become of particular interest and clinically useful in genetically-defined cancers. Notably, by targeting synthetic lethality in the context of BRCA1 or BRCA2 deficiency, so-called PARP inhibitors (PARPi) are used to treat homologous-recombination (HR)-deficient and other cancers. This is thought to occur by lowering PARP activity and/or by biochemically “trapping” of PARP-1/2/3 on chromatin (Murai et al., 2012, 2014). While “trapping” remains molecularly ill-defined, the term defines an enhanced recruitment, association and/or retention of PARP-1/2/3 enzymes on damaged chromatin, typically induced by treatment of PARP-1 enzyme with PARP inhibitors (PARPi), or a decrease in the release of PARP-1/2/3 enzymes following their initial recruitment, which leads to a prolonged retention. This biochemically manifests itself in an enhanced steady-state association/retention/binding (“trapping”) of the PARP enzymes with damaged genome regions/loci.

Due to the clinical advent of PARPi, PARP-1 has emerged as a powerful target for an increasing list of cancers, including in combination with immuno-oncology therapies, such as the Lynparza/Keytruda trials, to give all but one of many examples. Moreover, first-line PARPi therapies and applications in contexts outside of germline BRCA-1/2 mutations are becoming possible.

Importantly, clinical PARPi compounds all bind essentially the same location at the catalytic center of the active site by blocking the binding of the substrate NAD⁺, thus preventing poly(ADP-ribose) synthesis, largely by virtue of their structural similarity to nicotinamide, a moiety of the NAD⁺ nucleotide.

Yet, PARPi exhibit greatly different clinical efficacy in tumor killing and patient outcomes in the clinic. One fundamental difference in the action of these PARPi is that they promote highly distinct levels of PARP-1/-2 trapping on chromatin. It is currently generally thought that the most powerful and clinically effective PARPi trap PARP-1 at the site of a DNA break much more strongly than clinically less useful PARPi. Upon PARP trapping, DNA lesions become more cytotoxic, especially in mutant tumor cells with genetic or epigenetic deficiencies in the repair of DNA strand breaks, such as functionally HR-deficient BRCA-1/2 mutant tumor cells. Further, it is currently not clear what relative or dominant contributions are carried out by PARP-1 vs. PARP-2 vs. PARP-3 enzymes, since all enzymes are involved in sensing DNA strand breaks and recruit to DNA damage sites, while PARP-1 and PARP-2 both promote PARylation of chromatin factors, while all existing clinical PARPi molecules barely distinguish between the two related PAR polymerase enzymes PARP-1 and PARP-2. In turn, PARP trapping is thought to lead to DNA replication stress, genomic instability and cell death in cancer cells (Lord and Ashworth, 2012). Enhanced trapping of PARP1, for example, is thought to lead to an increased ability to kill cancer cells, especially cancers with defective DNA repair pathways (Zandarashvili et al., 2020). FIG. 1 shows the cytotoxic mechanism of PARP trapping via PARPi.

“PARP trapping” is thus described as an (enhanced) association of PARP-1 and/or PARP-2 and/or PARP-3 with chromatin in living cells. Using PARPi, the allosteric mechanism that contributes to binding of PARP-1 to DNA can be disrupted (Zandarashvili et al., 2020), with some PARPi contributing to retention and others facilitating pro-release mechanisms based on in vitro PARP-1, PARPi and DNA interactions (Zandarashvili et al., 2020). U2OS cells treated with talazoparib show an enhanced retention of GFP-tagged PARP1 and PARP2 at induced DNA lesions, whereas cells treated with veliparib reveal overall less PARP1/PARP2 recruitment to the DNA lesions.

Either PARP-1 or PARP-2 are necessary for a sufficient recruitment of SSB repair proteins to the damage site. Via PARylation, chromatin remodeling or histone-modifying enzymes are activated at DNA damage sites, which leads to changes in chromatin compaction (Luijsterburg et al., 2016; Mehrotra et al., 2011; Sellou et al., 2016; Smeenk et al., 2013; Timinszky et al., 2009).

Activation of PARP-1 and/or PARP-2 enzymes leads to the recruitment of many proteins, notably also specific chromatin remodeling enzymes, notably including the macrodomain-containing nucleosome remodeler ALC1 (CHD1L) (Ahel et al., 2009; Gottschalk et al., 2009; Lehmann et al., 2017; Singh et al., 2017). Macrodomains generally bind ADP-ribose, oligo-ADP-ribose and poly-ADP-ribose (Karras et al., 2005), thus proteins containing macrodomains respond and recruit to PARP activation sites on the genome, including during DNA damage and with relevance for cancer. Importantly, PAR or oligo-ADP-ribose binding to the macrodomains of ALC1 robustly turns on chromatin remodeling activity (Ahel et al., 2009; Gottschalk et al., 2009; Lehmann et al., 2017; Singh et al., 2017), revealing ALC1 as an allosterically-regulated chromatin remodeling enzyme, the first of its kind, and one of the very few enzymes whose catalytic activity is directly regulated by PAR. Additionally, ALC1 is a validated oncogene and is often genetically amplified together with PARPI in BRCA1/2-deficient ovarian and breast cancer samples (see FIG. 2). ALC1 inhibitors, such as small molecules inhibiting the ATPase function and or nucleosome remodeling functions of ALC1, could potentiate the effect PARPi, lead to enhanced cancer cell killing and/or reduce off-target effects and thus lessen cellular toxicity in non-cancer cells. The fact that altering the expression level of ALC1 (by a CRISPR-based knockout) impacts the sensitivity of cancer cells to PARP inhibitors, also opens up the opportunity that altering the activity levels of ALC1 could overcome PARP inhibitor resistance, since removing ALC1 robustly potentiates the PARPi Olaparib to a level that may be sufficient to circumvent PARPi resistance, such as upon (but not limited to) reversion of the BRCA-deficiency status (e.g. by internal deletions or through loss of epigenetic BRCA1/2 gene silencing). Since the sensitivity of cancer cells to PARP inhibitors is generally accepted to (also) stem from the ability of PARPi to trap PARP1 on chromatin (and likely also trapping PARP2 enzymes—this has not been formally established in the field), we hypothesized that small molecule ALC1 inhibitors may mediate PARPi sensitization. Specifically, we hypothesized that inhibition of ALC1 with small molecules would promote DNA damage accumulation and cancer cell killing.

Since ALC1 is upregulated in several tumors and is a validated oncogene in hepatocellular carcinoma (Cheng et al., 2013; Li et al., 2019; Su et al., 2014), inhibition of ALC1 via small molecule inhibitors could drive robust changes in DNA damage responses.

The present inventors hypothesized that manipulation of ALC1 activity via small molecules could induce strong anti-proliferative effects and in addition be sufficient to bypass a low or high level of PARPi resistance. Thus, in addition to a potential use as a monotherapy, combined ALC1 and PARP-1/2 inhibition could be exploited to refine PARP-targeted therapies in oncology.

This hypothesis led to a part of the present invention that ALC1 manipulation via small molecule inhibitors impacts the response to DNA damage. These compounds, designed to inhibit the enzymatic activity of the ATP-dependent chromatin remodeler ALC1 (CHD1L), will potentiate accumulation of DNA damage and thus to mediate synthetic lethality upon BRCA deficiency or more generally, in HRD high tumors, since PARP inhibition has been well documented to be synthetic lethal with loss of BRCA-1/2 tumor suppressor function. Based on preclinical and clinical evidence, ALC1 inhibition could therefore serve as an additional therapeutic approach for cancers that have intact HR pathways, and/or allow the targeting of ALC1-amplified tumors by disrupting ALC1's oncogene function. Also, since the ALC1 gene is a key mediator of PARP-chromatin rearrangements upon induced DNA damage (Sellou et al., 2016), small molecules targeting ALC1 activity may impact the nuclear, DNA-damage relevant functions of PARP-1/2/3 on chromatin rearrangements without impacting other roles of PARP-1/2/3 inside or outside of the cell's nucleus or without impacting non-DNA-damage induced PARP enzymes, which may result in a “second-generation PARPi” with reduced off-target effects and/or reduced side-effects.

Given the above-described relevance of ALC1 for various proliferative diseases, in particular those that are BRCA1/2 deficient, the present invention provides a novel class of compounds to treat or ameliorate tumor diseases and in particular tumor diseases characterized by increased activity of ALC1, e.g. due to increased expression.

Furthermore, the present inventors determined that by using a combination of PARPi and an ALC1 inhibitor, preferably the allosteric ALC1 inhibitors of the present invention, the effect of PARPis can surprisingly be enhanced. Thus, the present invention provides inter alia (i) an efficient therapy of tumors that are HRD deficient and hence sensitive to PARPi, (ii) mediate PARPi sensitization, (iii) bypass PARPi resistance, (iv) and/or allow the reduction of the amount of PARPi that is administered.

### SUMMARY OF THE INVENTION

In a first aspect, the present invention is directed at an inhibitor of ALC1 (ALC1i) according to formula (I)

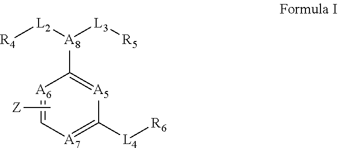

wherein
A5 and A8 are each independently selected from N or CH;
A6 is selected from N or CH, or when A6 takes part in the annulated carbo- or heterocycle Z, then A6 is C;
A7 is selected from N or CH, or when A7 takes part in the annulated carbo- or heterocycle Z, then A7 is C;
L2 is selected from the group consisting of —CH₂—R4, —CF₂—R4, —CH₂—CH₂—R4, —CH₂—CH₂—CH₂—R4, —O—R4, —NH—R4, —N═R4;
L3 is selected from the group consisting of CH₂—R5, —CF₂—R5, —CH₂—CH₂—R5, —CH₂—CF₂—R5, —CH₂—CH₂—CH₂—R5, —O—R5, —NH—R5, —N═R5;
   or
L2 and L3 together with the A8 to which they are connected form a 5- or 6-membered heterocycle substituted by R4 and/or R5;
L4 is CH₂, —CF₂—, CH₂—CH₂, CH₂—CH₂—CH₂, O, N, and NH, or is absent;
Z is a 5-, 6- or 7-membered carbo- or heterocycle, optionally substituted with one, two, or three, (preferably one) substituent(s) selected from the group consisting of —Br, —Cl, —F, —I, —OH, Me, —CF₃, Et, —OMe, —SMe, and —NO₂;
   and can be annulated to the central core or connected via a covalent bond
R4 is 5-, 6- or 7-membered carbo- or heterocycle, optionally substituted with one, two, or three, (preferably one) substituent(s) selected from the group consisting of —Br, —Cl, —F, —I, —CF₃, Me, Et, —OMe, and —SMe, or R4 is hydrogen, methyl, or COOH;
R5 is a 4-, 5-, 6-, 7-, 8-, 9- or 10-membered carbo- or heterocycle, optionally substituted with one, two, or three (preferably one) substituent(s) selected from the group consisting of —Br, —Cl, —F, —I, Me, —CF₃, Et, —OMe, and —SMe;
R6 is a 5-, 6- or 7-membered carbo- or heterocycle, optionally substituted with one, two, or three (preferably one) substituent(s) selected from the group consisting of —Br, —Cl, —F, —I, —OH, —NO₂, Me, —CF₃, Et, —OMe, and —SMe;
   or R6 is H;
or
when A7 takes part in the annulated carbo- or heterocycle Z then A5 and A6 are independently selected from —N or —CH and A8 is selected from —N, —CH, —CH₂—N, —CH₂—CH, or —NH—CH;
or pharmaceutically acceptable salt thereof,
for treating or ameliorating a proliferative disease in a patient.

In a second aspect the present invention relates to a bifunctional compound comprising the ALC1i for use as specified for the first aspect of the invention and a compound which recruits E3 ubiquitin ligase to ALC1(E3 recruiter), wherein the ALC1i and the E3 recruiter are covalently linked, optionally through a linker.

In a third aspect the present invention relates to a pharmaceutical composition comprising the ALC1i for use of the first aspect of the invention or the bifunctional compound of the second aspect of the invention and one or more pharmaceutically acceptable excipients.

In a fourth aspect the present invention relates to an ALC1i for use of the first aspect of the invention or the bifunctional compound of the second aspect of the invention for use in treating or ameliorating a proliferative disease, wherein the treating and ameliorating the proliferative disease comprises the administration of said ALC1i or of said bifunctional compound and the administration of a Poly(ADP-ribose)-Polymerase inhibitor (PARPi).

In a fifth aspect the present invention relates to a PARPi for use in treating or ameliorating a proliferative disease in a patient, wherein the treating and ameliorating the proliferative disease comprises the administration of said PARPi and the administration of the ALC1i for use of the first aspect of the invention or of the bifunctional compound of the second aspect of the invention.

In a sixth aspect the present invention relates to a kit of parts comprising separately packaged a PARPi and an ALC1i for use of the first aspect of the invention or the bifunctional compound of the second aspect of the invention or a composition comprising a PARPi and an ALC1i, preferably with instructions for use to treat or ameliorate a proliferative disease.

### DESCRIPTION OF THE FIGURES

In the following, the content of the Figures comprised in this specification is described. In this context please also refer to the detailed description of the invention above and/or below.

FIG. 1: Cytotoxic mechanisms of PARPi on DNA repair pathways leading to PARP trapping. Upper pathway shows interference with DNA repair of single strand breaks (SSBs) via DNA replication fork damage leading to repair via the homologous recombination (HR) mechanism. Lower pathway shows trapping of PARP1/2 proteins on damaged DNA, leading to replication fork damage utilizing additional repair pathways including Fanconi pathway (FA), template switching (TS), ATM, FENI (replicative flap endonuclease) and DNA polymerase f (Murai et al., 2012, S. 5591).

FIG. 2: The chromatin remodeler ALC1 (CHD1L) is frequently co-amplified with the PARP1 gene in human ovarian and breast cancer samples. Genomic alterations of ALC1 (CHD1L), PARPI, PARP2, BRCA1, BRCA2 and the most closely related chromatin remodeler CHD1 among the genomes of 10792 breast, fallopian and ovarian cancer patients (OncoPrint analysis conducted on Aug. 12, 2020 at the cBioPortal—www.cbioportal.org). The percentage numbers indicate the percentage of alterations in a particular gene for all genomes where the specific gene has been profiled. Gene amplifications (black), deep gene deletions (dark grey) are highlighted. Compared to CHD1, ALC1 is often amplified together with PARP1 (ALC1-PARP1: Odd's ratio (OR)=1.581, q-value=<0.001; CHD1-PARP1: OR=−0.125, q-value=0.250). Neither ALC1, nor PARP1 exhibit a deep deletion or frequent mutation when the tumors exhibit a deep deletion or mutation in the BRCA1 or BRCA2 tumor suppressor genes (ALC1-BRCA1/2: OR=<−3/-2.178, q-value=<0.001; PARP1-BRCA1/2: OR=<−3, q-value=<0.001).

FIG. 3: 96 hour SRB assay of BRCA positive and BRCA negative cells treated with ALCi. MDA-MB-231 cells (BRCA1/2 wildtype) and SUM-149-PT cells (BRCA1 negative) cells were seeded into 96-well plates and treated with titrations of different ALCi starting at 50 μM. The cells were cultured at 37° C., CO₂ 5% for 4 days, fixed with 10% TCA and stained with sulforhodamine staining to analyse cell survival. The data was normalized to DMSO controls indicating 100% survival. Inhibitor vs. response curves with variable slope (four parameters) were fitted using GraphPad Prism. Standard deviation bars are shown for two technical replicates.

FIG. 4: PARPi co-treatment Cell proliferation assay of pancreatic cancer cells. PSN-1 cells were seeded into 96-well plates and treated with a 2-D titration of PARPi vs. ALC1i. The cells were cultured at 37° C., CO₂ 5% for 4 days, fixed with 10% TCA and stained with Sulforhodamine staining to analyze cell survival. Single agent titration curves are shown on the left, a dose response matrix is shown in the middle and a synergy score calculation is shown on the right. Treatment with ALC1i-1002 and Talazoparib, Olaparib, or Niraparib shows most synergsistic area scores of 14.28, 13.27, and 15.41 respectively. ZIP synergy scores over 0.0 are an indication of strong synergy.

FIG. 5: Structures and associated compound codes of ALC1 inhibitors.

FIG. 6: Nucleosome remodeling inhibition. IC₅₀s (μM) of ALC1 inhibitors in a FRET based nucleosome remodelling assay. IC₅₀ values are presented by the following symbols: +++: IC₅₀<25 μM; ++: IC₅₀=25-100 μM; +: IC₅₀=>100-250 μM; and ‘-’: compound is not active, i.e. never observe inhibition >50%.

FIG. 7: Cell proliferation inhibition EC50s (μM) of ALC1 inhibitors in a 4 day cell proliferation assay with an SRB based readout. EC₅₀ values are presented by the following symbols: +++: EC₅₀<10 μM; ++: EC₅₀=10-25 μM; +: EC₅₀=>25-50 μM; and ‘-’: compounds is not active, i.e. never observe inhibition >50%.

FIG. 8: A table containing the supplier used for each of the inhibitors.

FIG. 9: A general synthesis scheme for inhibitors according to formula I.

FIG. 10: A general synthesis scheme for inhibitors according to formula I.

FIG. 11: A table containing the percentage inhibition of ALC1 in the FRET-based nucleosome sliding assay at a compound concentration of 250 μM for each of the inhibitors. For some compounds, the concentration of 250 μM had to be lowered due to solubility problems as indicated in the legend at the bottom of the table.

FIG. 12: EC50 values of 96 hour SRB assay and 11 days colony formation assay of HR proficient (HRP) and HR deficient (HRD) cells treated with ALCi. Different cancer cell lines with a variation of deleterious mutations (downloaded from https://depmap.org/portal/) in the homologous recombination repair pathways (HR) were seeded into 96-well plates and treated with titrations of different ALCi starting at 50 μM. The cells were cultured at 37° C., CO₂ 5% for 4 days (96h) or 11 days (colony formation), fixed with 10% TCA and stained with sulforhodamine staining to analyse cell survival. The data was normalized to DMSO controls indicating 100% survival. Inhibitor vs. response curves with variable slope (four parameters) were fitted using GraphPad Prism. Standard deviation bars are shown for two technical replicates.

FIG. 13: List of genes involved in DNA Damage Response (DDR) (Human DNA Repair Genes, n.d.) cited in Wood R D, Mitchell M, & Lindahl T Mutation Research, 2005, in Science, 2001, in the reference book DNA Repair and Mutagenesis, 2nd edition, 2006, and in Nature Reviews Cancer, 2011 (modified by R. Wood and M. Lowery on Wednesday 10 Jun. 2020). FIG. 14: list of genes related to the homologous recombination repair pathway (HR). Summary of genes from Toh & Ngeow, 2021; Kim et al., 2021; Yamamoto & Hirasawa, 2021 and the HRD-signature genes from Pent et al, 2021.

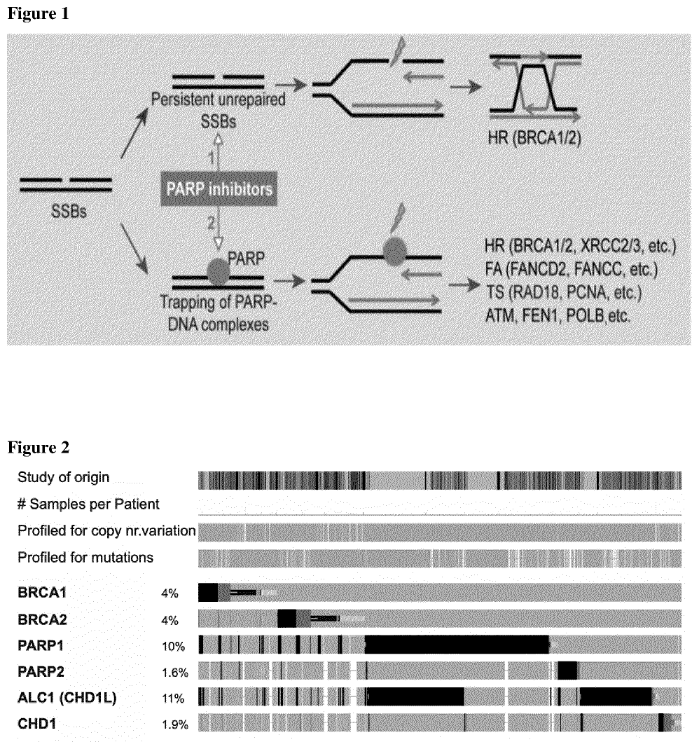

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

To practice the present invention, unless otherwise indicated, conventional methods of chemistry, biochemistry, and recombinant DNA techniques are employed which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, some definitions of terms frequently used in this specification are provided. These terms will, in each instance of its use, in the remainder of the specification have the respectively defined meaning and preferred meanings.

As used in this specification and the appended claims, the singular forms “a”, “an”, and “the” include plural referents, unless the content clearly dictates otherwise.

The term “Chromodomain-helicase-DNA-binding protein 1-like” abbreviated CHD1L refers to a protein that is also termed ALC1. The amino acid sequence of human ALC1 is as specified in SEQ ID NO: 1. The 897 amino acid residues long protein consists of an N-terminal Snf2-like DNA dependent ATPase domain spanning amino acid residues 40 to 513, which contains the conserved helicase motifs critical for catalysis (Flaus et al., 2006). This domain is composed of two RecA like lobes ranging from amino acid residues 48 to 261 and 351 to 513, respectively. The structure of a truncated N-terminal lobe of the ATPase domain has been determined by homology modeling in order to identify putative allosteric binding sites, a minimal coordinate file of this model is provided as FIG. 20 to allow the skilled person to identify model compounds within the allosteric binding pocket defined for the first time by the present inventors. The allosteric binding pocket is spatially separated from that part of ALC1 involved in binding ATP. The ATPase domain is followed by a linker region ranging from amino acid residues 514 to 703, which contains a putative coiled-coil region (amino acid residues 638 to 675), and a C-terminal macrodomain (amino acid residues 704 to 897). The macrodomain has been shown to directly interact with the ATPase domain, thereby inhibiting its catalytic function (Lehmann et al., 2017; Singh et al., 2017). This interaction is released upon poly(ADP-ribose) binding to the macrodomain, leading to an activation of the chromatin remodelling enzyme.

The term “alkyl” as used in the context of the present invention refers to a saturated straight or branched carbon chain. Preferably, the chain comprises from 1 to 10 carbon atoms, i.e. 1, 2, 3, 4, 5, 6, 7, 8, 9 or 10, e.g. methyl, ethyl propyl (n-propyl or iso-propyl), butyl (n-butyl, iso-butyl, sec-butyl, tert-butyl), pentyl, hexyl, heptyl, octyl, nonyl, decyl. Alkyl groups are optionally substituted.

The term “heteroalkyl” as used in the context of the present invention refers to a saturated straight or branched carbon chain. Preferably, the chain comprises from 1 to 9 carbon atoms, i.e. 1, 2, 3, 4, 5, 6, 7, 8, or 9, e.g. methyl, ethyl, propyl, iso-propyl, butyl, iso-butyl, sec-butyl, tert-butyl, pentyl, hexyl, heptyl, octyl, nonyl, which is interrupted one or more times, e.g. 1, 2, 3, 4, 5, with the same or different heteroatoms. Preferably, the heteroatoms are selected from O, S, and N, e.g. —(CH₂)ₙ—X—(CH₂)ₘCH₃, with n=0, 1, 2, 3, 4, 5, 6, 7, 8, or 9, m=0, 1, 2, 3, 4, 5, 6, 7, 8, or 9 and X═S, O or NR′ with R′═H or hydrocarbon (e.g. C₁ to C₆ alkyl). In particular “heteroalkyl” refers to —O—CH₃, —OC₂H₅, —CH₂—O—CH₃, —CH₂—O—C₂H₅, —CH₂—O—C₃H₇, —CH₂—O—C₄H₉, —CH₂—O—C₅H₁₁, —C₂H₄O—CH₃, —C₂H₄—O—C₂H₅, —C₂H₄—O—C₃H₇, —C₂H₄—O—C₄H₉ etc. Heteroalkyl groups are optionally substituted.

The term “haloalkyl” refers to a saturated straight or branched carbon chain in which one or more hydrogen atoms are replaced by halogen atoms, e.g. by fluorine, chlorine, bromine or iodine. Preferably, the chain comprises from 1 to 10 carbon atoms, i.e. 1, 2, 3, 4, 5, 6, 7, 8, 9 or 10. In particular, “haloalkyl” refers to —CH₂F, —CHF₂, —CF₃, —C₂H₄F, —C₂H₃F₂, —C₂H₂F₃, —C₂HF₄, —C₂F₅, —C₃H₆F, —C₃H₅F₂, —C₃H₄F₃, —C₃H₃F₄, —C₃H₂F₅, —C₃HF₆, —C₃F₇, —CH₂Cl, —CHCl₂, —CCl₃, —C₂H₄Cl, —C₂H₃Cl₂, —C₂H₂Cl₃, —C₂HCl₄, —C₂Cl₅, —C₃H₆Cl, —C₃H₅Cl₂, —C₃H₄Cl₃, —C₃H₃Cl₄, —C₃H₂Cl₅, —C₃HCl₆, and —C₃Cl₇. Haloalkyl groups are optionally substituted.

The term “carbocycle” is used in the context of the present invention to refer to mono-, bi or tricyclic “cycloalkyl”, “cycloalkenyl”, “spiroalkyl” “sprioalkenyl” or “aryl” wherein the ring is formed by carbon atoms. Carbocycle groups are optionally substituted.

The term “5, 6, or 7 membered carbocycle” is used in the context of the present invention to refer to “cycloalkyl”, “cycloalkenyl”, “spiroalkyl” or “aryl”, preferably “cycloalkyl”, or “cycloalkenyl” with 5, 6, or 7 carbon atoms forming a ring or phenyl.

The term “cycloalkyl” includes cyclopentyl, cyclohexyl, and cycloheptyl. Cycloalkyl groups are optionally substituted.

The term “cycloalkenyl” includes cyclopentenyl, cyclohexenyl, and cycloheptenyl. Cycloalkenyl groups are optionally substituted.

The term “aryl” refers to phenyl. Aryl is optionally substituted, e.g. naphthyl.

The term “heterocycle” is used in the context of the present invention to refer to mono or bicyclic “heterocycloalkyl” or mono- or bicyclic “heteroaryl”; wherein at least one of the carbon atoms are replaced 1, 2, or 3 for the monocyclic heterocycle or 1, 2, 3, or 4 for the bicyclic heterocycle of the same or different heteroatoms, preferably selected from O, N and S.

The term “5, 6, or 7 membered heterocycle” is used in the context of the present invention to refer to monocyclic “5, 6, or 7 membered heterocycloalkyl” or monocyclic “5, 6, or 7 membered heteroaryl” with 5, 6, or 7 atoms forming a ring.

The term “5, 6, or 7 membered heterocycloalkyl” refers to a saturated monocycle, wherein at least one of the carbon atoms are replaced by 1, or 2 (for the five membered ring) or 1, 2, or 3 (for the six membered ring) or 1, 2, 3, or 4 (for the seven membered ring) of the same or different heteroatoms, preferably selected from O, N and S. Preferred examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, or 2-piperazinyl. Heterocycloalkyl groups are optionally substituted.

The term “heteroaryl” as used in the context of the present invention refers to a 5, 6 or 7-membered aromatic monocyclic ring wherein at least one of the carbon atoms are replaced by 1, 2, or 3 (for the five membered ring) or 1, 2, 3, or 4 (for the six membered ring) of the same or different heteroatoms, preferably selected from O, N and S. Examples of preferred heteroaryls are furanyl, thienyl, oxazolyl, isoxazolyl, 1,2,5-oxadiazolyl, 1,2,3-oxadiazolyl, pyrrolyl, imidazolyl, pyrazolyl, 1,2,3-triazolyl, thiazolyl, isothiazolyl, 1,2,3,-thiadiazolyl, 1,2,5-thiadiazolyl, pyridinyl, pyrimidinyl, pyrazinyl, 1,2,3-triazinyl, 1,2,4-triazinyl, 1,3,5-triazinyl. Heteroaryls groups are optionally substituted.

If two or more radicals can be selected independently from each other, then the term “independently” means that the radicals may be the same or may be different.

The term “optionally substituted” in each instance if not further specified refers to halogen (in particular F, Cl, Br, or I), —NO₂, —CN, —OR′″, —NR′R″, —COOR′″, —CONR′R″, —NR′COR″, —NR″COR′″, —NR′CONR′R″, —NR′SO₂E, —COR′″; —SO₂NR′R″, —OOCR′″, —CR′″R″″OH, —R′″OH, and -E;
R′ and R″ is each independently selected from the group consisting of hydrogen, alkyl, alkenyl, alkynyl, cycloalkyl, heterocycloalkyl, aryl, aralkyl, and heteroaryl or together form a heteroaryl, or heterocycloalkyl;
R′″ and R″″ is each independently selected from the group consisting of hydrogen, alkyl, alkenyl, alkynyl, cycloalkyl, heterocycloalkyl, alkoxy, aryl, aralkyl, heteroaryl, and —NR′R″;
E is selected from the group consisting of alkyl, alkenyl, alkynyl, cycloalkyl, alkoxy, alkoxyalkyl, heterocycloalkyl, an alicyclic system, aryl and heteroaryl; optionally substituted.

“Pharmaceutically acceptable” means approved by a regulatory agency of the Federal or a state government or listed in the U.S. Pharmacopeia (United States Pharmacopeia-33/National Formulary-28 Reissue, published by the United States Pharmacopeia Convention, Inc., Rockville Md., publication date: April 2010) or other generally recognized pharmacopeia for use in animals, and more particularly in humans.

The term “pharmaceutically acceptable salt” refers to a salt of a compound of the present invention. Suitable pharmaceutically acceptable salts of the compound of the present invention include acid addition salts which may, for example, be formed by mixing a solution of a compound described herein or a derivative thereof with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compound of the invention carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include but are not limited to: acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxyethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide a compound of formula (I) to (IV), and especially a compound shown in FIG. 14. A prodrug is an active or inactive compound that is modified chemically through in vivo physiological action, such as hydrolysis, metabolism and the like, into a compound of this invention following administration of the prodrug to a patient. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme. The suitability and techniques involved in making and using prodrugs are well known by those skilled in the art. For a general discussion of prodrugs involving esters, see Svensson L. A. and Tunek A. (1988) Drug Metabolism Reviews 19(2): 165-194 and Bundgaard H. “Design of Prodrugs”, Elsevier Science Ltd. (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bundgaard H. et al. (1989) J. Med. Chem. 32(12): 2503-2507). Also, drugs containing an acidic NH group, such as imidazole, imide, indole and the like, have been masked with N-acyloxymethyl groups (Bundgaard H. “Design of Prodrugs”, Elsevier Science Ltd. (1985)). Hydroxy groups have been masked as esters and ethers. EP 0 039 051 A2 discloses Mannich-base hydroxamic acid prodrugs, their preparation and use.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

As used herein, “para position” when referring to the substituent of an aryl means that the substituent occupies the position opposite to the position at which the aryl is linked to the backbone of the compound.

As used herein, a “patient” means any mammal or bird that may benefit from a treatment with the compounds described herein. Preferably, a “patient” is selected from the group consisting of laboratory animals, domestic animals, or primates including chimpanzees and human beings. It is particularly preferred that the “patient” is a human being.

As used herein, “treat”, “treating” or “treatment” of a disease or disorder means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, “prevent”, “preventing”, “prevention”, or “prophylaxis” of a disease or disorder means preventing that a disorder occurs in a subject for a certain amount of time. For example, if a compound described herein is administered to a subject with the aim of preventing a disease or disorder, said disease or disorder is prevented from occurring at least on the day of administration and preferably also on one or more days (e.g. on 1 to 30 days; or on 2 to 28 days; or on 3 to 21 days; or on 4 to 14 days; or on 5 to 10 days) following the day of administration.

A “pharmaceutical composition” according to the invention may be present in the form of a composition, wherein the different active ingredients and diluents and/or carriers are admixed with each other, or may take the form of a combined preparation, where the active ingredients are present in partially or totally distinct form. An example for such a combination or combined preparation is a kit-of-parts.

An “effective amount” is an amount of a therapeutic agent sufficient to achieve the intended purpose. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

The term “carrier”, as used herein, refers to a diluent, adjuvant, excipient, or vehicle with which the therapeutic agent is administered. Such pharmaceutical carriers can be sterile liquids, such as saline solutions in water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatine, malt, rice flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

### Embodiments of the Invention

The present inventors have identified and characterized ALC1 inhibitors that appear to be involved in allosteric regulation of the nucleosome sliding activity of ALC1. These compounds specifically bind to an allosteric pocket and are capable of inhibiting activity of ALC1. Compounds that bind to the ATPase site of ALC1 and block the ATPase activity have to compete with ATP for binding to the ATPase site. Since the cellular ATP concentration is in the range of 1 to 10 mM depending on the cellular compartment, very high binding affinities in the low nanomolar range are required to successfully prevent ATP from binding to the ATPase site of ALC1. Allosteric inhibitors of ALC1 do not have this limitation since they do not have to prevent ATP from binding but inhibit ALC1's ATPase activity through a different mechanism. The present inventors have identified compounds that are capable of specifically binding to an allosteric pocket of ALC1. These compounds were also tested for their ability to kill two different tumor cell lines one of which was BRCA deficient.

Accordingly, in a first aspect the present invention relates to the use of an ALC1i of formula (I):

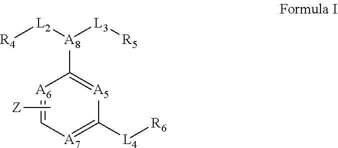

wherein
A5 and A8 are each independently selected from N or CH;
A6 is selected from N or CH, or when A6 takes part in the annulated carbo- or heterocycle Z, in particular a 5, 6, or 7 membered carbo- or heterocycle, then A6 is C;
A7 is selected from N or CH, or when A7 takes part in the annulated carbo- or heterocycle Z, in particular a 5, 6, or 7 membered carbo- or heterocycle, then A7 is C;
L2 is selected from the group consisting of —CH₂—R4, —CF₂—R4, —CH₂—CH₂—R4, —CH₂—CH₂—CH₂—R4, —O—R4, —NH—R4, —N═R4;
L3 is selected from the group consisting of CH₂—R5, —CF₂—R5, —CH₂—CH₂—R5, —CH₂—CF₂—R5, —CH₂—CH₂—CH₂—R5, —O—R5, —NH—R5, —N═R5;
   or
L2 and L3 together with the A8 to which they are connected form a 5- or 6-membered heterocycle substituted by R4 and/or R5;
L4 is CH₂, —CF₂—, CH₂—CH₂, CH₂—CH₂—CH₂, 0, N, and NH, or is absent;
Z is a 5-, 6- or 7-membered carbo- or heterocycle, optionally substituted with one, two, or three, preferably one substituent(s) selected from the group consisting of —Br, —Cl, —F, —I, —OH, Me, —CF₃, Et, —OMe, —SMe, and —NO₂;
   and can be annulated to the central core or connected via a covalent bond
R4 is 5-, 6- or 7-membered carbo- or heterocycle, optionally substituted with one, two, or three, preferably one substituent(s) selected from the group consisting of —Br, —Cl, —F, —I, —CF₃, Me, Et, —OMe, and —SMe, or R4 is hydrogen, methyl, or COOH;
R5 is a 4-, 5-, 6-, 7-, 8-, 9- or 10-membered carbo- or heterocycle, optionally substituted with one, two, or three (preferably one) substituent(s) selected from the group consisting of —Br, —Cl, —F, —I, Me, —CF₃, Et, —OMe, and —SMe;
R6 is a 5-, 6- or 7-membered carbo- or heterocycle, optionally substituted with one, two, or three (preferably one) substituent(s) selected from the group consisting of —Br, —Cl, —F, —I, —OH, —NO₂, Me, —CF₃, Et, —OMe, and —SMe;
   or R6 is H;
or
when A7 takes part in the annulated carbo- or heterocycle Z then A5 and A6 are independently selected from —N or —CH and A8 is selected from —N, —CH, —CH₂—N, —CH₂—CH, or —NH—CH; or pharmaceutically acceptable salt thereof
for treating or ameliorating a proliferative disease in a patient.

The ALC1i according to formula (I) preferably shows inhibition >50% at concentrations at or below 250 μM, preferably has an IC₅₀ of <250 μM, and more preferably of <25 μM. The IC₅₀ is preferably measured in a FRET based nucleosome remodeling assay as described in the Examples.

The ALC1i according to formula (I) preferably has an EC₅₀ of <250 μM, preferably of <50 μM; and more preferably of <10 μM. The EC₅₀ is preferably measured in cell proliferation assay with an SRB based readout as described in the Examples.

In an embodiment of the first aspect it is preferred that:
A5 and A6 are N; and
A7 takes part in the annulated carbo- or heterocycle Z, in particular a 5, 6, or 7 membered carbo- or heterocycle Z, preferably carbocycle Z, in particular a 5, 6, or 7 membered carbocycle Z, optionally substituted with one, two, or three, preferably one substituent(s) selected from the group consisting of —Br, —Cl, —F, —I, —OH, Me, —CF₃, Et, —OMe, —SMe, and —NO₂.

In an embodiment of the first aspect it is preferred that:
A5 and A6 are N;
A7 takes part in the annulated carbo- or heterocycle, preferably carbocycle Z, optionally substituted with one, two, or three, preferably one substituent(s) selected from the group consisting of —Br, —Cl, —F, —I, —OH, Me, —CF₃, Et, —OMe, —SMe, and —NO₂; and
A8 is —CH₂—N, —CH₂—CH, or —NH—CH, preferably —NH—CH.

In an embodiment of the first aspect it is preferred that:
A5 is N and A8 is —N or —CH.

In an embodiment of the first aspect it is preferred that:
A5 is N and A8 is —N or —CH; and
A6 takes part in the annulated carbo- or heterocycle, preferably carbocycle Z.

In a particularly preferred embodiment A5, A7 and A8 are N.

In a particularly preferred embodiment A5, A7 and A8 are N and A6 takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂.

In a particularly preferred embodiment A5, A7 and A8 are N and A6 takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂.

In an embodiment of the first aspect it is preferred that:
L2 is selected from the group consisting of —CH₂—R4, —CH₂—CH₂—R4, —CH₂—CH₂—CH₂—R4.

In an embodiment of the first aspect it is preferred that:
L2 is selected from the group consisting of —CH₂—R4, —CF₂—R4, —CH₂—CH₂—R4, —CH₂—CH₂—CH₂—R4, —NH—R4; and
R4 is 6-membered aryl or 5-, 6- or 7-membered heteroaryl, preferably 5- or 6-membered heteroaryl, optionally substituted with one, two, or three, preferably one substituent(s) selected from the group consisting of —Br, —Cl, —F, —I, —CF₃, Me, Et, —OMe, and —SMe, or R4 is hydrogen, methyl, or COOH.

In an embodiment of the first aspect it is preferred that:
L3 is selected from the group consisting of —CH₂—R5, —CH₂—CH₂—R5, and —CH₂—CF₂—R5.

In an embodiment it is preferred that:
L3 is selected from the group consisting of —CH₂—R5, —CH₂—CH₂—R5, —CH₂—CF₂—R5; and
R5 is a 4-, 5-, 6-, 7-, 8-, 9- or 10-membered carbo- or heterocycle, optionally substituted with one, two, or three (preferably one) substituent(s) selected from the group consisting of —Br, —Cl, —F, —I, Me, —CF₃, Et, —OMe, and —SMe.

In an embodiment of the first aspect it is preferred that:
L2 and L3 together with the A8 to which they are connected form a 5- or 6-membered heterocycle substituted by R4 and R5.

In an embodiment of the first aspect it is preferred that:
L2 and L3 together with the A8 to which they are connected form a 5- or 6-membered heterocycle substituted by R4 and R5;
R4 is hydrogen, methyl, or COOH; and
R5 is a 4-, 5-, 6-, 7-membered carbo- or heterocycle, preferably phenyl or 5- or 6-membered heteroaryl, optionally substituted with one, two, or three, preferably one substituent(s) selected from the group consisting of —Br, —Cl, —F, —I, Me, —CF₃, Et, —OMe, and —SMe.

In an embodiment of the first aspect it is preferred that
L4 is absent.

In a particularly preferred embodiment A5, A7 and A8 are N; and
L4 is absent.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂; and
L4 is absent.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂; and
L4 is absent.

In a particularly preferred embodiment A5, A7 and A8 are N; and
L4 is absent
R6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂; and
L4 is absent
R6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂; and
L4 is absent
R6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N; and
L4 is absent
R6 is phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂;
L4 is absent; and
R6 is phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂;
L4 is absent; and
R6 is a phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, which is optionally substituted with one, two, or three, preferably one substituent(s) selected from the group consisting of —Br, —Cl, —F, —I, —OH, Me, —CF₃, Et, —OMe, —SMe, and —NO₂;
L4 is absent; and
R6 is phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl. which is optionally substituted with one, two, or three, preferably one substituent(s) selected from the group consisting of —Br, —Cl, —F, —I, —OH, Me, —CF₃, Et, —OMe, —SMe, and —NO₂;
L4 is absent; and
R6 is a phenyl, optionally substituted with one, two, or three, preferably one substituent(s) selected from the group consisting of —Br, —Cl, —F, —I, —OH, —NO₂, Me, —CF₃, Et, —OMe, and —SMe.

In an embodiment of the first aspect it is preferred that:
R4 is hydrogen, methyl, COOH or tetrazolyl.

In an embodiment of the first aspect it is preferred that:
L2 is —CH₂—R4, —CH₂—CH₂—R4, —CH₂—CH₂—CH₂—R4; and
R4 is hydrogen, methyl, COOH or tetrazolyl.

In an embodiment of the first aspect it is preferred that:
R5 is any 4-, 5-, 6- 7-, 8-, 9-, or 10-membered carbo- or heterocycle, optionally substituted with one, two, or three, preferably one substituent(s) selected from the group consisting of —Br, —Cl, —F, —I, —CF₃, Me, Et, —OMe, and —SMe.

In an embodiment of the first aspect it is preferred that:
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe.

In an embodiment of the first aspect it is preferred that:
L3 is selected from the group consisting of —CH₂—R5, —CH₂—CH₂—R5, —CH₂—CF₂—R5; and
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe.

In an embodiment of the first aspect it is preferred that:
R6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe
   or R6 is H.

In an embodiment of the first aspect it is preferred that:
L4 is absent; and
R6 is a 5-, or 6-membered carbo- or heterocycle, preferably 6-membered carbo- or heterocycle, optionally substituted with one, two, or three (preferably one) substituents selected from the group consisting of —Br, —Cl, —F, —I, —OH, —NO₂, Me, —CF₃, Et, —OMe, and —SMe.

In above embodiment it is preferred that:
Z is a 6 membered aryl, or a 5-, 6-membered heteroaryl, preferably phenyl, optionally substituted with one, two, or three, preferably one substituent(s) selected from the group consisting of —Br, —Cl, —F, —I, —OH, Me, —CF₃, Et, —OMe, —SMe, and —NO₂.

In an embodiment of the first aspect, it is preferred that:
A5 is N;
one of A6 and A7 is CH and the other one is N; or one of A6 and A7 is C and takes part in the annulated carbo- or heterocycle Z and the other one is N;
A8 is N or CH;
L2, L3, L4 are independently from each other selected from the group consisting of CH₂, —CF₂—, CH₂—CH₂, CH₂—CH₂—CH₂, 0, N, and NH, or are absent, or L2 and L3 together with the A8 to which they are connected form a 5- or 6-membered heterocycle substituted by R4 and R5;
Z is any 5-, 6- or 7-membered carbo- or heterocycle and can be annulated to the central core or connected via a covalent bond and optionally substituted with one, two, or three (preferably one) substituent(s)
selected from the group consisting of —Br, —Cl, —F, —I, —OH, Me, —CF₃, Et, —OMe, —SMe, and —NO₂;
R4 is any 5-, 6- or 7-membered carbo- or heterocycle, optionally substituted with one, two, or three (preferably one) substituent(s) selected from the group consisting of —Br, —Cl, —F, —I, —CF₃, Me, Et, —OMe, and —SMe, or R4 is hydrogen, methyl, or COOH;
R5 is a 4-, 5-, 6- 7-, 8-, 9-, or 10-membered carbo- or heterocycle, optionally substituted with one, two, or three (preferably one) substituents selected from the group consisting of —Br, —Cl, —F, —I, —CF₃, Me, Et, —OMe, and —SMe;
R6 is a 5-, 6- or 7-membered carbo- or heterocycle, optionally substituted with one, two, or three (preferably one) substituent(s) selected from the group consisting of —Br, —Cl, —F, —I, —OH, —NO₂, Me, —CF₃, Et, —OMe, and —SMe,
   or R6 is H
or
when A7 takes part in the annulated carbo- or heterocycle Z then A5 and A6 are N and A8 is selected from —N, —CH, —CH₂—N, —CH₂—CH, or —NH—CH, preferably —CH₂—N, —CH₂—CH, or —NH—CH;
wherein R4, R5, and R6.

In an embodiment of the first aspect it is preferred that:
A5 is N;
one of A6 and A7 is C and takes part in the annulated carbo- or heterocycle Z and the other one is N;
A8 is N;
L2 is CH₂—CH₂ and L3 is CH₂—CH₂ or CH₂—CF₂; or L2 and L3 together with the A8 to which they are connected form a 5- or 6-membered heterocycle (preferably piperidine or a pyrrolidine) substituted by R4 and R5;
L4 is absent;
Z is a 6-membered carbo- or heterocycle annulated to the central core, wherein Z is optionally substituted with one, two, or three (preferably one) substituents selected from the group consisting of —Br, —Cl, —F, —I, —OH, Me, —CF₃, Et, —OMe, —SMe, and NO₂;
R4 is COOH or CH₂N₄;
R5 is a 4-, 5-, 6-, 7-, or 10-membered carbo- or heterocycle, optionally substituted with one, two, or three, preferably one) substituent(s) selected from the group consisting of —Br, —Cl, —F, —I, Me, —CF₃, Et, —OMe, and —SMe; and
R6 is a 5- or 6-membered carbo- or heterocycle, optionally substituted with one, two, or three, preferably one substituent(s) selected from the group consisting of —Br, —Cl, —F, —I, —OH, —NO₂, Me, —CF₃, Et, —OMe, and —SMe,
   or R6 is H.

In an embodiment of the first aspect it is preferred that:
each one of A5, A7 and A8 is N;
A6 is C and takes part in the annulated carbo- or heterocycle Z, preferably 5-, 6- or 7-membered carbo- or heterocycle, optionally substituted with one, two, or three (preferably one) substituent(s) selected from the group consisting of —Br, —Cl, —F, —I, —OH, —NO₂, Me, —CF₃, Et, —OMe, and —SMe;
L2 is CH₂—CH₂—R4;
L3 is CH₂—CH₂—R4 or —CH₂—CF₂—R5;
or L2 and L3 together with the A8 to which they are connected form a piperidine ring or a pyrrolidine ring, substituted by R4 and R5;
L4 is absent;
Z is phenyl or cyclohexyl annulated to the central core, wherein Z is optionally substituted with one, two, or three, preferably one substituent(s) selected from the group consisting of —Br, —Cl, —F, —OH, Me, —CF₃, —OMe, and —NO₂,
R4 is COOH or tetrazolyl;
R5 is phenyl, cyclobutyl, cyclopentyl or adamantyl, optionally substituted with one, two, or three, preferably one substituent(s) selected from the group consisting of —Br, —Cl, —CF₃, Me, —CH₂—CF₃, and —OMe;
R6 is a 6-membered carbo- or heterocycle, preferably phenyl or cyclohexyl; more preferably phenyl, optionally substituted with one, two, or three, preferably one substituent(s) selected from the group consisting of Br, —Cl, —F, Me, —CF₃, —OMe, and —NO₂.

In a particularly preferred embodiment A5, A7 and A8 are N;
L2 is —CH₂—R4, —CH₂—CH₂—R4, —CH₂—CH₂—CH₂—R4;
R4 is hydrogen, methyl, COOH or tetrazolyl, preferably COOH; and
L4 is absent.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂;
L2 is —CH₂—R4, —CH₂—CH₂—R4, —CH₂—CH₂—CH₂—R4;
R4 is hydrogen, methyl, COOH or tetrazolyl, preferably COOH; and
L4 is absent.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂; and
L4 is absent.

In a particularly preferred embodiment A5, A7 and A8 are N;
L2 is —CH₂—R4, —CH₂—CH₂—R4, —CH₂—CH₂—CH₂—R4;
R4 is hydrogen, methyl, COOH or tetrazolyl, preferably COOH;
L4 is absent; and
R6 is a phenyl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂;
L2 is —CH₂—R4, —CH₂—CH₂—R4, —CH₂—CH₂—CH₂—R4;
R4 is hydrogen, methyl, COOH or tetrazolyl, preferably COOH;
L4 is absent; and
R6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂;
L2 is —CH₂—R4, —CH₂—CH₂—R4, —CH₂—CH₂—CH₂—R4;
R4 is hydrogen, methyl, COOH or tetrazolyl, preferably COOH;
L4 is absent; and
R6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N;
L2 is —CH₂—R4, —CH₂—CH₂—R4, —CH₂—CH₂—CH₂—R4;
R4 is hydrogen, methyl, COOH or tetrazolyl, preferably COOH;
L4 is absent; and
R6 is phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂;
L2 is —CH₂—R4, —CH₂—CH₂—R4, —CH₂—CH₂—CH₂—R4;
R4 is hydrogen, methyl, COOH or tetrazolyl, preferably COOH;
L4 is absent; and
R6 is phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂;
L2 is —CH₂—R4, —CH₂—CH₂—R4, —CH₂—CH₂—CH₂—R4;
R4 is hydrogen, methyl, COOH or tetrazolyl, preferably COOH;
L4 is absent; and
R6 is a phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, which is optionally substituted with one, two, or three, preferably one substituent(s) selected from the group consisting of —Br, —Cl, —F, —I, —OH, Me, —CF₃, Et, —OMe, —SMe, and —NO₂;
L2 is —CH₂—R4, —CH₂—CH₂—R4, —CH₂—CH₂—CH₂—R4;
R4 is hydrogen, methyl, COOH or tetrazolyl, preferably COOH;
L4 is absent; and
R6 is phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl. which is optionally substituted with one, two, or three, preferably one substituent(s) selected from the group consisting of —Br, —Cl, —F, —I, —OH, Me, —CF₃, Et, —OMe, —SMe, and —NO₂;
L2 is —CH₂—R4, —CH₂—CH₂—R4, —CH₂—CH₂—CH₂—R4;
R4 is hydrogen, methyl, COOH or tetrazolyl, preferably COOH;
L4 is absent; and
R6 is a phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N;
L4 is absent; and
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂;
L4 is absent; and
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂;
L4 is absent; and
R5 is C₅ to C₇-cycloalkyl, i.e. C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe.

In a particularly preferred embodiment A5, A7 and A8 are N;
L4 is absent;
R5 is C₅ to C₇-cycloalkyl, i.e. C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂;
L4 is absent;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂;
L4 is absent;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N;
L4 is absent;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂;
L4 is absent;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂;
L4 is absent;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is a phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, which is optionally substituted with one, two, or three, preferably one substituent(s) selected from the group consisting of —Br, —Cl, —F, —I, —OH, Me, —CF₃, Et, —OMe, —SMe, and —NO₂;
L4 is absent;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl. which is optionally substituted with one, two, or three, preferably one substituent(s) selected from the group consisting of —Br, —Cl, —F, —I, —OH, Me, —CF₃, Et, —OMe, —SMe, and —NO₂;
L4 is absent;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is a phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N; and
L3 is selected from the group consisting of —CH₂—R5, —CH₂—CH₂—R5, and —CH₂—CF₂—R5
L4 is absent; and
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂;
L3 is selected from the group consisting of —CH₂—R5, —CH₂—CH₂—R5, and —CH₂—CF₂—R5;
L4 is absent; and
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂; and
L3 is selected from the group consisting of —CH₂—R5, —CH₂—CH₂—R5, and —CH₂—CF₂—R5;
L4 is absent; and
R5 is C₄ to C₇-cycloalkyl, i.e. C₄-, i.e. C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe.

In a particularly preferred embodiment A5, A7 and A8 are N;
L3 is selected from the group consisting of —CH₂—R5, —CH₂—CH₂—R5, and —CH₂—CF₂—R5;
L4 is absent;
R5 is C₄ to C₇-cycloalkyl, i.e. C₄-, i.e. C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂;
L3 is selected from the group consisting of —CH₂—R5, —CH₂—CH₂—R5, and —CH₂—CF₂—R5;
L4 is absent; and
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂;
L3 is selected from the group consisting of —CH₂—R5, —CH₂—CH₂—R5, and —CH₂—CF₂—R5;
L4 is absent; and
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMeR6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N;
L3 is selected from the group consisting of —CH₂—R5, —CH₂—CH₂—R5, and —CH₂—CF₂—R5;
L4 is absent;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂; and
L3 is selected from the group consisting of —CH₂—R5, —CH₂—CH₂—R5, and —CH₂—CF₂—R5;
L4 is absent;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂; and
L3 is selected from the group consisting of —CH₂—R5, —CH₂—CH₂—R5, and —CH₂—CF₂—R5;
L4 is absent;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is a phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, which is optionally substituted with one, two, or three, preferably one substituent(s) selected from the group consisting of —Br, —Cl, —F, —I, —OH, Me, —CF₃, Et, —OMe, —SMe, and —NO₂; and
L3 is selected from the group consisting of —CH₂—R5, —CH₂—CH₂—R5, and —CH₂—CF₂—R5;
L4 is absent; and
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe
R6 is phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl. which is optionally substituted with one, two, or three, preferably one substituent(s) selected from the group consisting of —Br, —Cl, —F, —I, —OH, Me, —CF₃, Et, —OMe, —SMe, and —NO₂; and
L3 is selected from the group consisting of —CH₂—R5, —CH₂—CH₂—R5, and —CH₂—CF₂—R5;
L4 is absent;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is a phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N; and
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent; and
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂;
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent; and
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂; and
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent; and
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe.

In a particularly preferred embodiment A5, A7 and A8 are N;
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂;
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent; and
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂; and
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent; and
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMeR6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N;
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂; and
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂; and
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent;
R5 is C₄ to C₇-cycloalkyl, i.e. C₄-, i.e. C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is a phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, which is optionally substituted with one, two, or three, preferably one substituent(s) selected from the group consisting of —Br, —Cl, —F, —I, —OH, Me, —CF₃, Et, —OMe, —SMe, and —NO₂; and
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent; and
R5 is C₅ to C₇-cycloalkyl C₄ to C₇-cycloalkyl, i.e. C₄-, i.e. C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe
R6 is phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl. which is optionally substituted with one, two, or three, preferably one substituent(s) selected from the group consisting of —Br, —Cl, —F, —I, —OH, Me, —CF₃, Et, —OMe, —SMe, and —NO₂; and
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent;
R5 is C₄ to C₇-cycloalkyl, i.e. C₄-, i.e. C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is a phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N; and
L3 is selected from the group consisting of —CH₂—R5, —CH₂—CH₂—R5, and —CH₂—CF₂—R5
L4 is absent; and
R4 is hydrogen, methyl, COOH or tetrazolyl, preferably COOH;
R5 is C₄ to C₇-cycloalkyl, i.e. C₄-, i.e. C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂;
L3 is selected from the group consisting of —CH₂—R5, —CH₂—CH₂—R5, and —CH₂—CF₂—R5;
L4 is absent; and
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂; and
L3 is selected from the group consisting of —CH₂—R5, —CH₂—CH₂—R5, and —CH₂—CF₂—R5;
L4 is absent; and
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe.

In a particularly preferred embodiment A5, A7 and A8 are N;
L3 is selected from the group consisting of —CH₂—R5, —CH₂—CH₂—R5, and —CH₂—CF₂—R5;
L4 is absent;
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂;
L3 is selected from the group consisting of —CH₂—R5, —CH₂—CH₂—R5, and —CH₂—CF₂—R5;
L4 is absent; and
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂; and
L3 is selected from the group consisting of —CH₂—R5, —CH₂—CH₂—R5, and —CH₂—CF₂—R5;
L4 is absent; and
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMeR6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N;
L3 is selected from the group consisting of —CH₂—R5, —CH₂—CH₂—R5, and —CH₂—CF₂—R5;
L4 is absent;
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂; and
L3 is selected from the group consisting of —CH₂—R5, —CH₂—CH₂—R5, and —CH₂—CF₂—R5;
L4 is absent;
R4 is hydrogen, methyl, COOH or tetrazolyl, preferably COOH;
R5 C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂; and
L3 is selected from the group consisting of —CH₂—R5, —CH₂—CH₂—R5, and —CH₂—CF₂—R5;
L4 is absent;
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is a phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, which is optionally substituted with one, two, or three, preferably one substituent(s) selected from the group consisting of —Br, —Cl, —F, —I, —OH, Me, —CF₃, Et, —OMe, —SMe, and —NO₂; and
L3 is selected from the group consisting of —CH₂—R5, —CH₂—CH₂—R5, and —CH₂—CF₂—R5;
L4 is absent; and
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe
R6 is phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl. which is optionally substituted with one, two, or three, preferably one substituent(s) selected from the group consisting of —Br, —Cl, —F, —I, —OH, Me, —CF₃, Et, —OMe, —SMe, and —NO₂; and
L3 is selected from the group consisting of —CH₂—R5, —CH₂—CH₂—R5, and —CH₂—CF₂—R5;
L4 is absent;
R4 is hydrogen, methyl, COOH or tetrazolyl, preferably COOH;
R5 is C₄ to C₇-cycloalkyl, i.e. C₄-, i.e. C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is a phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N; and
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent; and
R4 is hydrogen, methyl, COOH or tetrazolyl, preferably COOH;
R5 is C₄ to C₇-cycloalkyl, i.e. C₄-, i.e. C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂;
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent; and
R4 is hydrogen, methyl, COOH or tetrazolyl, preferably COOH;
R5 is C₄ to C₇-cycloalkyl, i.e. C₄-, i.e. C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂; and
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent; and
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe.

In a particularly preferred embodiment A5, A7 and A8 are N;
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent;
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂;
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent; and
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂; and
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent; and
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMeR6 is a 6 membered aryl or 5-, 6- or 7-membered heteroaryl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N;
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent;
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂;
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent;
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂; and
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent;
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is a phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl, which is optionally substituted with one, two, or three, preferably one substituent(s) selected from the group consisting of —Br, —Cl, —F, —I, —OH, Me, —CF₃, Et, —OMe, —SMe, and —NO₂; and
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent; and
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe
R6 is phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment A5, A7 and A8 are N and A6 is C and takes part in the annulated carbocycle Z, in particular a 5, 6, or 7 membered carbocycle Z, preferably phenyl. which is optionally substituted with one, two, or three, preferably one substituent(s) selected from the group consisting of —Br, —Cl, —F, —I, —OH, Me, —CF₃, Et, —OMe, —SMe, and —NO₂; and
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent;
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is a phenyl, optionally substituted with one, two, or three, preferably one substituent(s) selected from the group consisting of —Br, —Cl, —F, —I, —OH, —NO₂, Me, —CF₃, Et, —OMe, and —SMe.

In a particularly preferred embodiment of the above embodiments:
R6 is phenyl ortho substituted with a substituent selected from the group consisting of —Br, —Cl, —F, —I, —OH, —NO₂, Me, —CF₃, Et, —OMe, and —SMe, preferably —Br, —Cl, —F, —I, more preferably Br.

In a particularly preferred embodiment of the above embodiments:
L4 is absent; and
R6 is phenyl ortho substituted with a substituent selected from the group consisting of —Br, —Cl, —F, —I, —OH, —NO₂, Me, —CF₃, Et, —OMe, and —SMe, preferably —Br, —Cl, —F, —I, more preferably Br.

In a particularly preferred embodiment A5, A7 and A8 are N;
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent;
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl ortho substituted with a substituent selected from the group consisting of —Br, —Cl, —F, —I, —OH, —NO₂, Me, —CF₃, Et, —OMe, and —SMe, preferably —Br, —Cl, —F, —I, more preferably Br.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂;
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent;
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl ortho substituted with a substituent selected from the group consisting of —Br, —Cl, —F, —I, —OH, —NO₂, Me, —CF₃, Et, —OMe, and —SMe, preferably —Br, —Cl, —F, —I, more preferably Br.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂;
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent;
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl ortho substituted with a substituent selected from the group consisting of —Br, —Cl, —F, —I, —OH, —NO₂, Me, —CF₃, Et, —OMe, and —SMe, preferably —Br, —Cl, —F, —I, more preferably Br

In a particularly preferred embodiment A5, A7 and A8 are N;
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent;
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl ortho substituted with a substituent selected from the group consisting of —Br, —Cl, —F, —I, —OH, —NO₂, Me, —CF₃, Et, —OMe, and —SMe, preferably —Br, —Cl, —F, —I, more preferably Br.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂;
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent;
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl ortho substituted with a substituent selected from the group consisting of —Br, —Cl, —F, —I, —OH, —NO₂, Me, —CF₃, Et, —OMe, and —SMe, preferably —Br, —Cl, —F, —I, more preferably Br.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂;
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent;
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl ortho substituted with a substituent selected from the group consisting of —Br, —Cl, —F, —I, —OH, —NO₂, Me, —CF₃, Et, —OMe, and —SMe, preferably —Br, —Cl, —F, —I, more preferably Br.

In a particularly preferred embodiment A5, A7 and A8 are N;
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent;
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl ortho substituted with a substituent selected from the group consisting of —Br, —Cl, —F, —I, —OH, —NO₂, Me, —CF₃, Et, —OMe, and —SMe, preferably —Br, —Cl, —F, —I, more preferably Br.

In a particularly preferred embodiment A5, A7 and A8 are N and A6 is C and takes part in the annulated carbo- or heterocycle Z, in particular a 5, 6, or 7 membered carbo- or heterocycle Z, preferably carbocycle Z, in particular a 5, 6, or 7 membered carbocycle Z, which optionally substituted with one, two, or three, preferably one substituent(s) selected from the group consisting of —Br, —Cl, —F, —I, —OH, Me, —CF₃, Et, —OMe, —SMe, and —NO₂;
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent;
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl ortho substituted with a substituent selected from the group consisting of —Br, —Cl, —F, —I, —OH, —NO₂, Me, —CF₃, Et, —OMe, and —SMe, preferably —Br, —Cl, —F, —I, more preferably Br.

In a particularly preferred embodiment A5, A7 and A8 are N and A6 is C and takes part in the annulated carbocycle Z, in particular a 5, 6, or 7 membered carbocycle Z, preferably phenyl, optionally substituted with one, two, or three, preferably one substituent(s) selected from the group consisting of —Br, —Cl, —F, —I, —OH, Me, —CF₃, Et, —OMe, —SMe, and —NO₂;
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent;
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl ortho substituted with a substituent selected from the group consisting of —Br, —Cl, —F, —I, —OH, —NO₂, Me, —CF₃, Et, —OMe, and —SMe, preferably —Br, —Cl, —F, —I, more preferably Br.

In a particularly preferred embodiment A5, A7 and A8 are N and A6 is C and takes part in the annulated carbocycle Z, in particular a 5, 6, or 7 membered carbocycle Z, preferably phenyl, which is optionally substituted with one, two, or three, preferably one substituent(s) selected from the group consisting of —Br, —Cl, —F, —I, —OH, Me, —CF₃, Et, —OMe, —SMe, and —NO₂;
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent;
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl ortho substituted with a substituent selected from the group consisting of —Br, —Cl, —F, —I, —OH, —NO₂, Me, —CF₃, Et, —OMe, and —SMe, preferably —Br, —Cl, —F, —I, more preferably Br.

In a particularly preferred embodiment A5, A7 and A8 are N and A6 is C and takes part in the annulated carbocycle Z, in particular a 5, 6, or 7 membered carbocycle Z, preferably phenyl. which is optionally substituted with one, two, or three, preferably one substituent(s) selected from the group consisting of —Br, —Cl, —F, —I, —OH, Me, —CF₃, Et, —OMe, —SMe, and —NO₂;
L2 is selected from the group consisting of —CH₂—R4, —CH₂—CH₂—R4, and —CH₂—CH₂—CH₂—R4;
L3 is selected from the group consisting of —CH₂—R5, —CH₂—CH₂—R5, and —CH₂—CF₂—R5;
L4 is absent;
R4 is hydrogen, methyl, COOH or tetrazolyl, preferably COOH;
R5 is C₄ to C₇-cycloalkyl, i.e. C₄-, C₅-, C₆- or C₇-cycloalkyl, C₆ to C₁₀-bicycloalkyl, i.e. C₆-, C₇-, C₈-, C₉- or C₁₀-bicycloalkyl, C₆ to C₁₀-spiroalkyl, i.e. C₆-, C₇-, C₈-, C₉- or C₁₀-spiroalkyl, phenyl, 5 or 6 membered heteroaryl, adamantyl, optionally substituted with one, two, or three, preferably one substituent(s) selected from the group consisting of —Br, —Cl, —F, —I, Me, —CF₃, Et, —OMe, and —SMe; more preferably cyclopentyl, cyclohexyl, phenyl, bromo-phenyl, bicyclo[2.2.1]heptyl, spiro[3,3]heptyl, or adamantyl, optionally substituted with one, two, or three, preferably one substituent(s) selected from the group consisting of —Br, —Cl, —F, —I, Me, —CF₃, Et, —OMe, and —SMe, more preferably unsubstituted phenyl or adamantyl substituted with one, or two substituent(s) selected from the group consisting of —Br, —Cl, —F, —I, Me, —CF₃, Et, —OMe, and —SMe; and
R6 is phenyl ortho substituted with a substituent selected from the group consisting of —Br, —Cl, —F, —I, —OH, —NO₂, Me, —CF₃, Et, —OMe, and —SMe, preferably —Br, —Cl, —F, —I, more preferably Br.

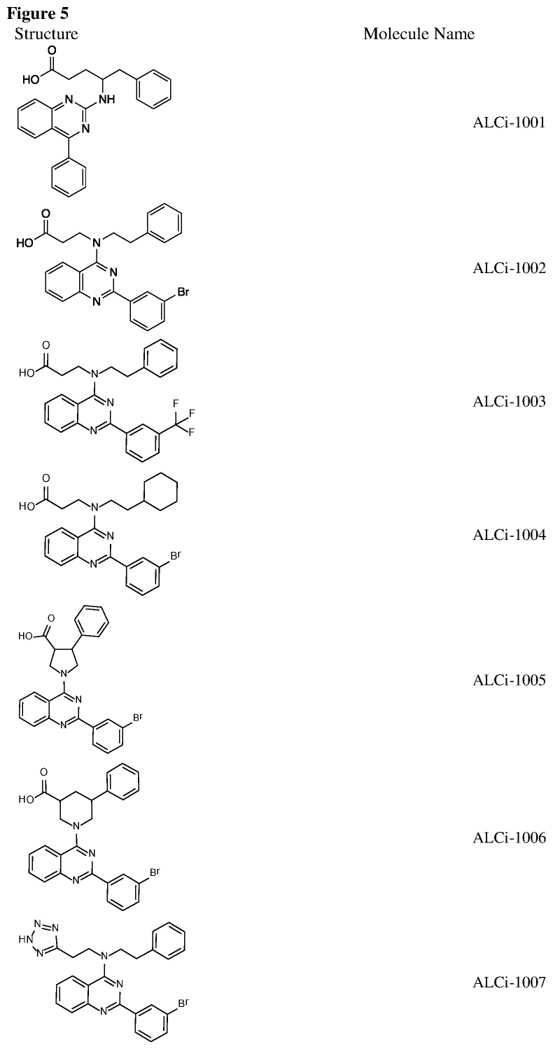

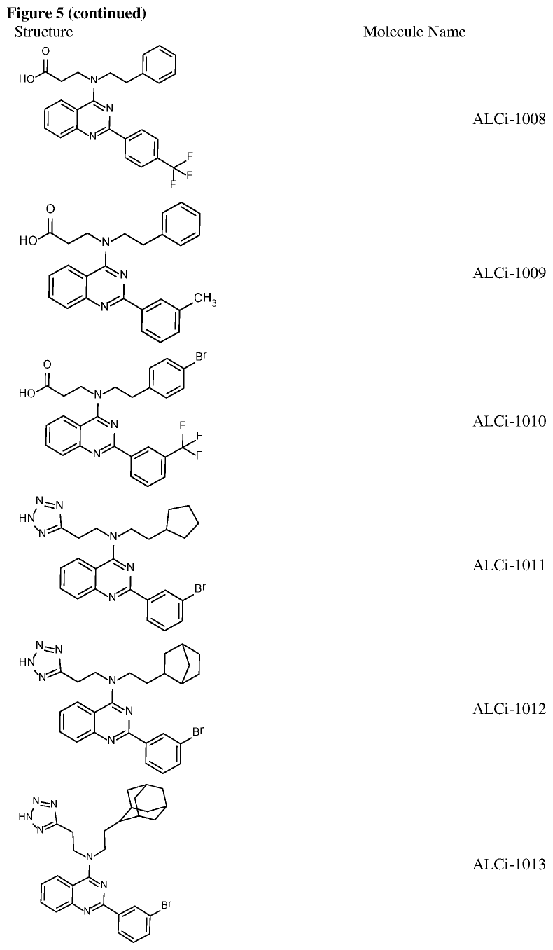

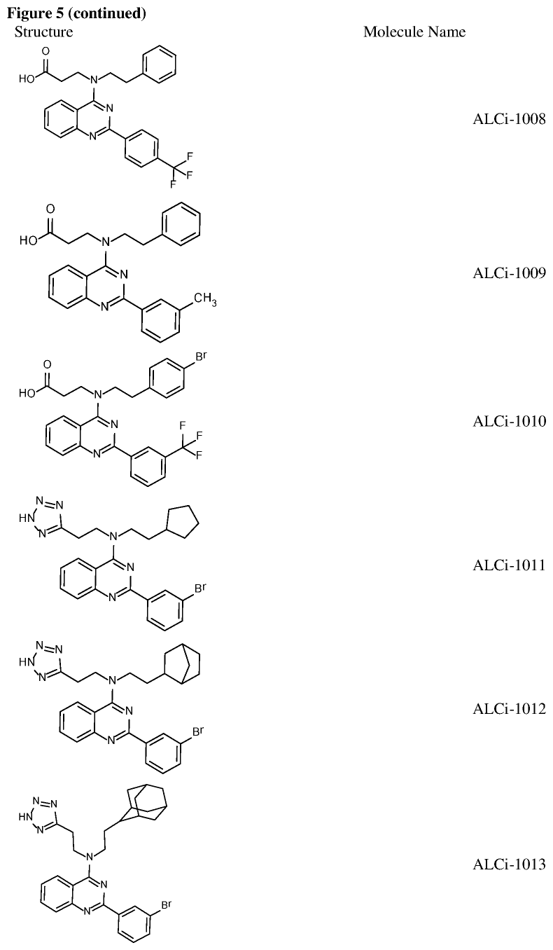

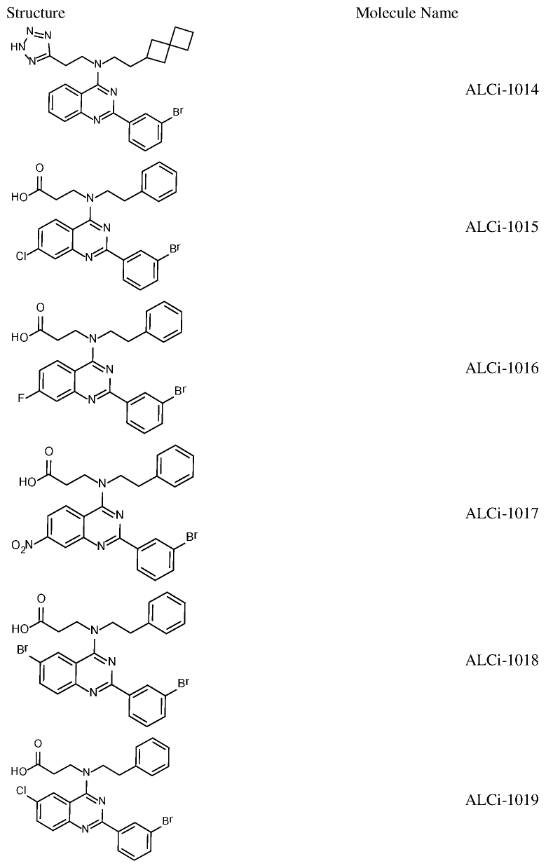

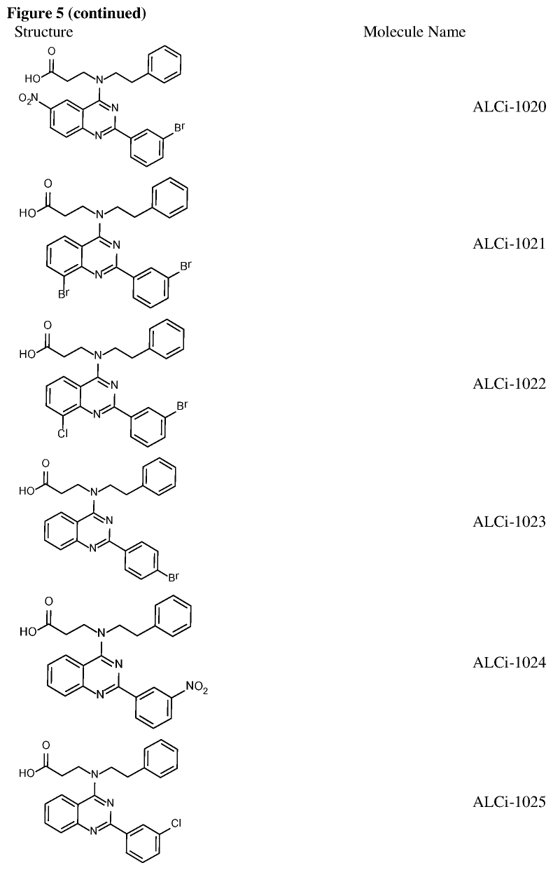

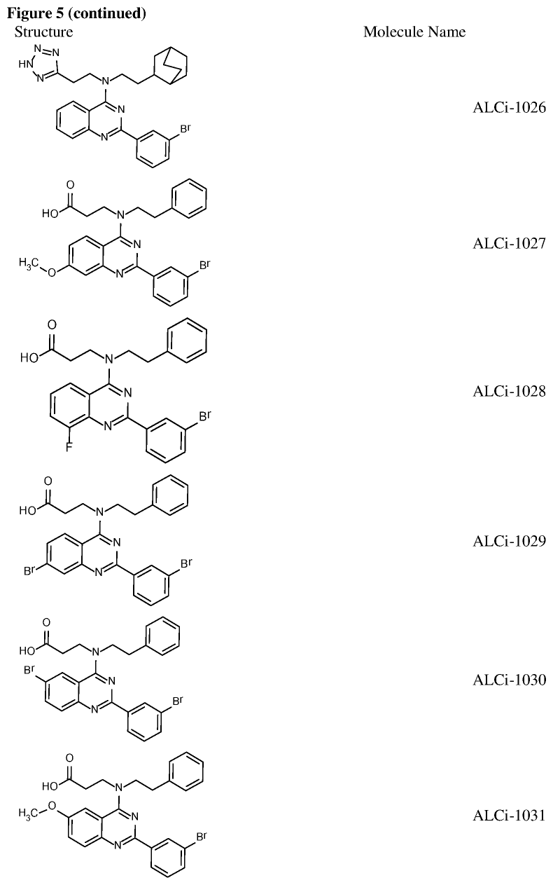

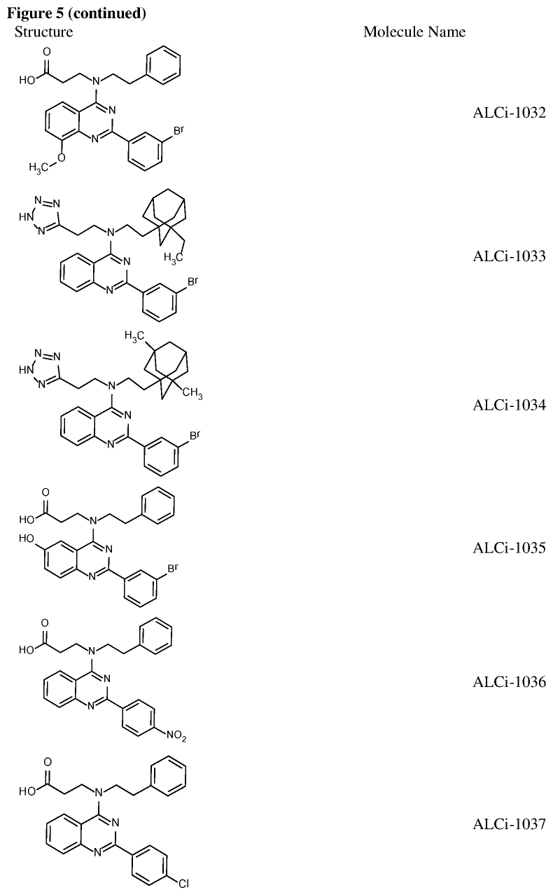

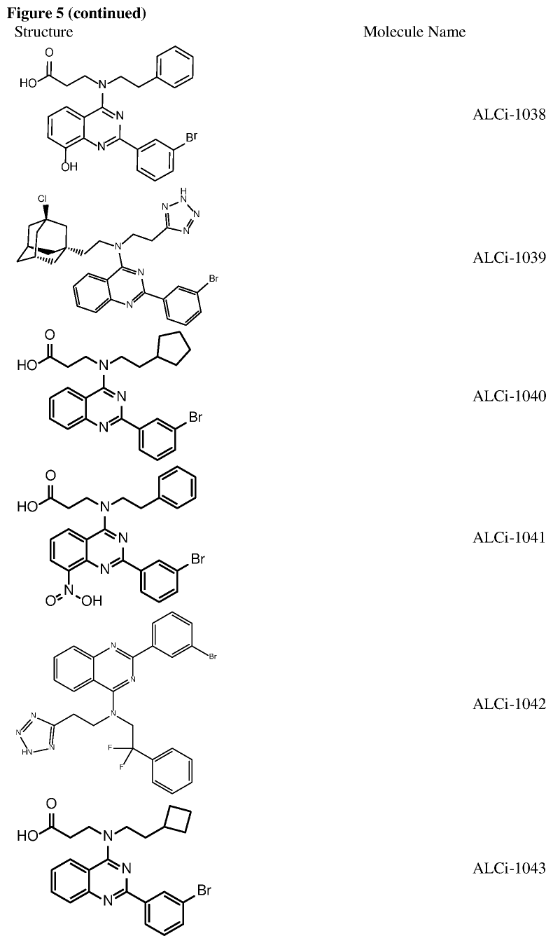

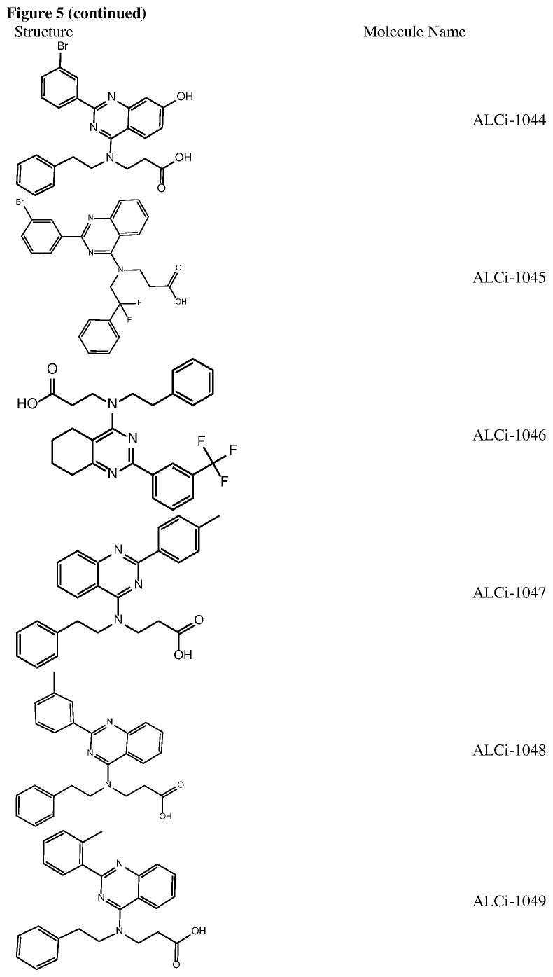

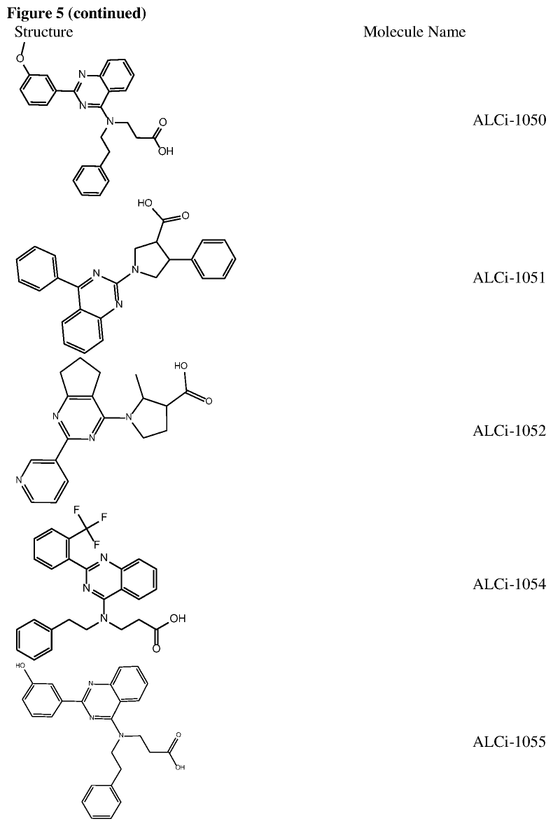

In a particularly preferred embodiment the compounds of the first and further aspect of the invention have the specific structures as indicated in FIG. 5.

The use of bifunctional compounds recruiting proteins involved in targeting proteins for degradation by the proteasome has emerged as a potential therapeutic strategy to degrade proteins that are involved in disease processes. This approach has met particular attention in cancer therapy (Khan S. et al., 2020 and Bushweller J H, 2019). Such bifunctional compounds are generally referred to as PROteolysis TArgeting Chimeras (PROTACs). The inhibitors of ALC1 of the first aspect of the invention specifically bind to ALC1 and are, thus suitable to recruit a protein that is part of the ubiquitination pathway to ALC1.

Accordingly, in a second aspect the present invention relates to a bifunctional compound comprising the allosteric inhibitor of ALC1 the first or further aspect of the present invention and a compound which recruits a protein that is part of the ubiquitination pathway to ALC1, preferably E3 ubiquitin ligase to ALC1(E3 recruiter), wherein the allosteric inhibitor of ALC1 and the E3 recruiter are covalently linked, optionally through a linker. In the context of the bifunctional compounds of the present invention that ability of the allosteric inhibitors of ALC1 of the first and further aspect of the invention to specifically bind to ALC1 is of particular importance. It is, thus preferred that the allosteric inhibitors of ACL1 bind to full length human ALC1 with an amino acid sequence according to SEQ ID NO: 1 with a KD of 50 PM, more preferably of 10 μM or lower, more preferably of 5 μM or lower, even more preferably of 1 μM, more preferably of 500 nM, more preferably of 200 nM and even more preferably of 100 nM or lower.

The protein of the ubiquitination pathway may either be bound by a small molecule or a protein ligand, e.g. an antibody or antibody-like protein, that specifically binds to a protein of the ubiquitination pathway. Such protein ligands have been described, for example in U.S. Pat. No. 7,223,556 Bi. Small molecules compounds that bind to a protein that is part of the ubiquitination pathway are well known in the art and can be used in the bifunctional compounds of the present invention. Examples of such molecules are described in EP 3 131 588, WO 2017/024317, U.S. Pat. Nos. 6,306,663, 7,041,298, US 2016/0176916, US 2016/0235730, US 2016/0235731, US 2016/0243247, WO 2016/105518, WO 2016/077380, WO 2016/105518, WO 2016/077375, WO 2017/007612, and WO 2017/024317.

In one embodiment the ALC1i is covalently linked to the compound which recruits a protein that is part of the ubiquitination pathway to ALC1. Preferably, the two components are covalently linked to each other through a linker. Suitable linkers have varying length and functionality. Preferably the linker is a carbon chain. In preferred embodiments, the carbon chain optionally comprises one, two, three, or more heteroatoms selected from N, O, and S. In preferred embodiments, the carbon chain comprises only saturated chain carbon atoms. In preferred embodiments, the carbon chain optionally comprises two or more unsaturated chain carbon atoms (e.g., C═C or C≡EC). In certain embodiments, one or more chain carbon atoms in the carbon chain are optionally substituted with one or more substituents, preferably oxo, C₁-C₆ alkyl, C₂-C₆ alkenyl, C₂-C₆ alkynyl, C₁-C₃ alkoxy, OH, halogen, NH₂, —NH(C₁-C₃ alkyl), —N(C₁-C₃ alkyl)₂, CN, C₃-C₇ cycloalkyl, heterocyclyl, phenyl, and heteroaryl).

In certain embodiments, the linker comprises at least 5 chain atoms (e.g., C, O, N, and S). In certain embodiments, the Linker comprises less than 20 chain atoms (e.g., C, O, N, and S). In certain embodiments, the Linker comprises 5, 6, 7, 8, 9, 10, 11, 12, 13, 14, 15, 16, 17, 18, or 19 chain atoms (e.g., C, O, N, and S). In certain embodiments, the Linker comprises 5, 7, 9, 11, 13, 15, 17, or 19 chain atoms (e.g., C, O, N, and S). In certain embodiments, the Linker comprises 5, 7, 9, or 11 chain atoms (e.g., C, O, N, and S). In certain embodiments, the Linker comprises 6, 8, 10, 12, 14, 16, or 18 chain atoms (e.g., C, O, N, and S). In certain embodiments, the Linker comprises 6, 8, 10, or 12 chain atoms (e.g., C, O, N, and S).

In certain embodiments, the Linker is a carbon chain optionally substituted with non-bulky substituents, preferably oxo, C₁-C₆ alkyl, C₂-C₆ alkenyl, C₂-C₆ alkynyl, C₁-C₃ alkoxy, OH, halogen, NH₂, —NH(C₁-C₃ alkyl), —N(C₁-C₃ alkyl)₂, CN, C₃-C₇ cycloalkyl, and CN).

In certain embodiments, the Linker is of Formula L(VI):

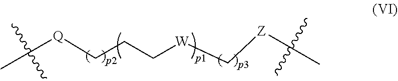

or an enantiomer, diastereomer, or stereoisomer thereof, wherein
p1 is an integer selected from 0 to 12;
p2 is an integer selected from 0 to 12;
p3 is an integer selected from 1 to 6;
each W is independently absent, CH₂, O, S, NH or NRS;
Z is absent, CH₂, O, NH or NRs;
each R₅ is independently H or C₁-C₃ alkyl, preferably C₁-C₃ alkyl; and
Q is absent or —CH₂C(O)NH—,
wherein the linker is covalently bonded to the compound that compound which recruits a protein that is part of the ubiquitination pathway with the bond that is next to Q and to the allosteric inhibitor of ALC1 with the bond that is next to Z, and wherein the total number of chain atoms in the linker is less than 20.

In a third aspect the present inventions relates to a pharmaceutical composition comprising the ALC1i as specified for the first aspect of the invention or the bifunction compound of the second aspect and at least one pharmaceutically acceptable excipient, preferably for use in treating and amelioration a proliferative disease.

In a fourth aspect the present inventions relates to an ALC1i for use of the first aspect of the invention or the bifunctional compound of the second aspect of the invention for use in treating or ameliorating a proliferative disease, wherein the treating and ameliorating of the proliferative disease comprises the administration of said ALC1i or of said bifunctional compound and the administration of a Poly(ADP-ribose)-Polymerase inhibitor (PARPi). The present inventors have discovered that the PARP trapping activity of known PARPi's can be enhanced by ALC1i. Due to this surprising synergy the combined use of PARPi's and ALC1i's for use of the first aspect of the invention in the treatment of various proliferative diseases is particularly advantageous.

The ALC1i may be provided to the physician administering the antiproliferative therapy separately from the PARPi or in a kit of parts. Thus, in the embodiments in which both are to be combined to obtain the benefit of the synergy the ALC1i may be provided with instructions to combine it with a PARPi of alternatively in a fifth aspect the PARPi may be provided with instructions to combine it with a ALC1i. Accordingly, in a fifth aspect the present invention relates to a PARPi for use in treating or ameliorating a proliferative disease in a patient, wherein the treating and ameliorating the proliferative disease comprises the administration of said PARPi and the administration of the ALC1i for use of the first aspect of the invention or of the bifunctional compound of the second aspect of the invention.

In a preferred embodiment of the ALC1i for use of the fourth aspect of the invention or of the PARPi for use of the fifth aspect of the invention the PARPi lowers PARP activity and/or inhibits PARP1, PARP2 and/or PARP3, preferably PARP2 on chromatin. The latter phenomenon is also referred to as PARP trapping. Thus, in a preferred embodiment PARP1, PARP2 and/or PARP3, preferably PARP2 is trapped.

In a preferred embodiment of the ALC1i for use of the fourth aspect of the invention or of the PARPi for use of the fifth aspect of the invention, the PARPi:
(i) that lowers PARP activity is selected from small interfering RNA, and
(ii) that inhibits PARPI is selected from the group consisting of a compound of
   (a) formula (II)

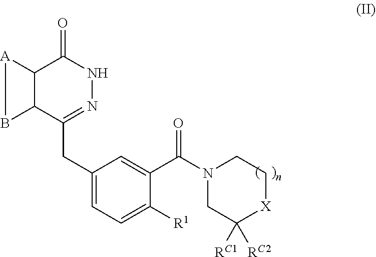

and isomers, salts, solvates, chemically protected forms, and prodrugs thereof wherein:
A and B together represent an optionally substituted, fused aromatic ring;
X is NR^{X} or CR^{X}R^{Y};
if X═NR^{X} then n is 1 or 2 and if X═CR^{X}R^{Y} then n is 1;
R^{X} is selected from the group consisting of H, optionally substituted C₁₋₂₀ alkyl, C₅₋₂₀ aryl, C₃₋₂₀ heterocyclyl, amido, thioamido, ester, acyl, and sulfonyl groups;
R^{Y} is selected from H, hydroxy, amino;
or R^{X} and R^{Y} may together form a spiro-C₃₋₇ cycloalkyl or heterocyclyl group;
R^{C1} and R^{C2} are independently selected from the group consisting of hydrogen and C₁₋₄ alkyl or when X is CR^{X}R^{Y}, R^{C1}, R^{C2}, R^{X} and R^{Y}, together with the carbon atoms to which they are attached, may form an optionally substituted fused aromatic ring; and
R¹ is selected from H and halo;
and
(b) formula (III)

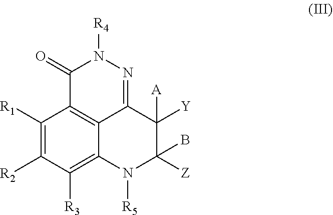

and isomers, salts, solvates, chemically protected forms, and prodrugs thereof wherein:
Y and Z are each independently selected from the group consisting of:
1. an aryl group optionally substituted with 1, 2, or 3 R₆;
2. a heteroaryl group optionally substituted with 1, 2, or 3 R₆;
3. a substituent independently selected from the group consisting of hydrogen, alkenyl (e.g. C₂₋₆-alkenyl), alkoxy (e.g. C₁₋₆-alkoxy), alkoxyalkyl (e.g. C₁₋₆-alkoxy-C₁₋₆-alkyl), alkoxycarbonyl (e.g. C₁₋₆-alkoxy-carbonyl), alkoxycarbonylalkyl (e.g. C₁₋₆-alkoxy-carbonyl-C₁₋₆-alkyl), alkyl (e.g. C₁₋₆-alkyl), alkynyl (e.g. C₂₋₆-alkynyl), arylalkyl (e.g. aryl-C₁₋₆-alkyl), cycloalkyl (e.g. C₃₋₈-cycloalkyl), cycloalkylalkyl (e.g. C₃₋₈-cycloalkyl-C₁₋₆-alkyl), haloalkyl (e.g. C₁₋₆-haloalkyl), hydroxyalkylene (e.g. hydroxy-C₁₋₆-alkylene), oxo, heterocycloalkyl (e.g. C₂₋₆-hetero cycloalkyl), heterocycloalkylalkyl (e.g. C₂₋₆-heterocycloalkyl-C₁₋₆-alkyl), alkylcarbonyl (e.g. C₁₋₆-alkyl-carbonyl), arylcarbonyl, heteroarylcarbonyl, alkylsulfonyl (e.g. C₁₋₆-alkyl-sulfonyl), arylsulfonyl, heteroarylsulfonyl, (R_{A}R_{B})alkylene (e.g. (R_{A}R_{B})-C₁₋₆-alkylene), (NR_{A}R_{B})carbonyl, (NR_{A}R_{B})carbonylalkylene (e.g. NR_{A}R_{B})carbonyl-C₁₋₆-alkylene), (NR_{A}R_{B})sulfonyl, and (R_{A}R_{B})sulfonylalkylene (e.g. (R_{A}R_{B})sulfonyl-C₁₋₆-alkylene);
wherein each R₆ is selected from OH, NO₂, CN, Br, Cl, F, I, C₁₋₆-alkyl, C₃₋₈-cycloalkyl, C₂₋₈-heterocycloalkyl; C₂₋₆-alkenyl, alkoxy (e.g. C₁₋₆-alkoxy), alkoxyalkyl (e.g. C₁₋₆-alkoxy-C₁₋₆-alkyl), alkoxycarbonyl (e.g. C₁₋₆-alkoxy-carbonyl), alkoxycarbonylalkyl (e.g. C₁₋₆-alkoxy-carbonyl-C₁₋₆-alkyl), C₂₋₆-alkynyl, aryl, arylalkyl (e.g. aryl-C₁₋₆-alkyl), C₃₋₈-cycloalkylalkyl (e.g. C₃₋₈-cycloalkyl-C₁₋₆-alkyl, haloalkoxy (e.g. C₁₋₆-haloalkoxy), haloalkyl (e.g. C₁₋₆-haloalkyl), hydroxyalkylene (e.g. hydroxy-C₁₋₆-alkylene), oxo, heteroaryl, heteroarylalkoxy (e.g. heteroaryl-C₁₋₆-alkoxy), heteroaryloxy, heteroarylthio, heteroarylalkylthio (e.g. heteroaryl-C₁₋₆-alkylthio), heterocycloalkoxy (e.g. C₂₋₆-heterocycloalkoxy), C₂₋₆-heterocycloalkylthio, heterocyclooxy, heterocyclothio, NR_{A}R_{B}, (R_{A}R_{B})C₁₋₆-alkylene, (NR_{A}R_{B})carbonyl, (R_{A}R_{B})carbonylalkylene (e.g. R_{A}R_{B})carbonyl-C₁₋₆-alkylene), (NR_{A}R_{B})sulfonyl, and (NR_{A}R_{B})sulfonylalkylene (e.g. (NR_{A}R_{B})sulfonyl-C₁₋₆-alkylene);
R₁, R₂, and R₃ are each independently selected from the group consisting of hydrogen, halogen, alkenyl (e.g. C₂₋₆-alkenyl), alkoxy (e.g. C₁₋₆-alkoxy), alkoxycarbonyl (e.g. C₁₋₆-alkoxy-carbonyl), alkyl (e.g. C₁₋₆-alkyl), cycloalkyl (e.g. C₃₋₈-cycloalkyl), alkynyl (e.g. C₂₋₆-alkynyl), cyano, haloalkoxy (e.g. C₁₋₆-haloalkoxy), haloalkyl (e.g. C₁₋₆-haloalkyl), hydroxyl, hydroxyalkylene (e.g. hydroxy-C₁₋₆-alkylene), nitro, NR_{A}R_{B}, NR_{A}R_{B} alkylene (e.g. NR_{A}R_{B} C₁₋₆-alkylene), and (R_{A}R_{B})carbonyl;
A and B are each independently selected from hydrogen, Br, Cl, F, I, OH, C₁₋₆-alkyl, C₃₋₈-cycloalkyl, alkoxy (e.g. C₁₋₆-alkoxy), alkoxyalkyl (e.g. C₁₋₆-alkoxy-C₁₋₆-alkyl), wherein C₁₋₆-alkyl, C₃₋₈-cycloalkyl, alkoxy, alkoxyalkyl are optionally substituted with at least one substituent selected from OH, NO₂, CN, Br, Cl, F, I, C₁₋₆alkyl, and C₃₋₈-cycloalkyl, wherein B is not OH;
R_{A}, and R_{B} are independently selected from the group consisting of hydrogen, alkyl (e.g. C₁₋₆-alkyl), cycloalkyl (e.g. C₃₋₈-cycloalkyl), and alkylcarbonyl (e.g. C₁₋₆-alkyl-carbonyl); or R_{A} and R_{B} taken together with the atom to which they are attached form a 3-10 membered heterocycle ring optionally having one to three heteroatoms or hetero functionalities selected from the group consisting
   of —O—, —NH, —N(C₁₋₆-alkyl)-, —NCO(C₁₋₆-alkyl)-, —N(aryl)-, —N(aryl-C₁₋₆-alkyl-), —N(substituted-aryl-C₁₋₆-alkyl-)-, —N(heteroaryl)-, —N(heteroaryl-C₁-C₆-alkyl-)-, —N(substituted-heteroaryl-C₁₋₆ alkyl-)-, and —S— or S(O)q-, wherein q is 1 or 2 and the 3-10 membered heterocycle ring is optionally substituted with one or more substituents;
R₄ and R₅ are each independently selected from the group consisting of hydrogen, alkyl (e.g. C₁₋₆-alkyl), cycloalkyl (e.g. C₃₋₈-cycloalkyl), alkoxyalkyl (e.g. C₁₋₆-alkoxy-C₁₋₆-alkyl), haloalkyl (e.g. C₁₋₆-haloalkyl), hydroxyalkylene (e.g. hydroxy-C₁₋₆-alkylene), and (NR_{A}R_{B})alkylene (e.g. NR_{A}R_{B} C₁₋₆-alkylene);
(iii) that inhibits PARPI and PARP2 is selected from the group consisting of a compound of
   (a) formula (IV)

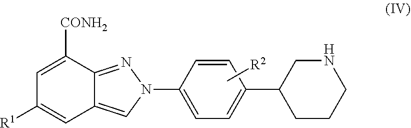

and isomers, salts, solvates, chemically protected forms, and prodrugs thereof wherein:
R₁ is hydrogen or fluorine; and
R₂ is hydrogen or fluorine;
and
(b) formula (V)

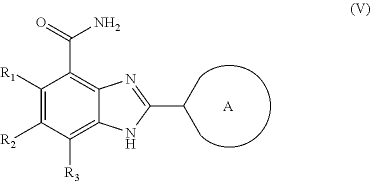

and isomers, salts, solvates, chemically protected forms, and prodrugs thereof wherein:
R₁, R₂, and R₃ are independently selected from the group consisting of hydrogen, alkenyl (e.g. C₁₋₆-alkenyl), alkoxy (e.g. C₁₋₆-alkoxy), alkoxycarbonyl (e.g. C₁₋₆-alkoxycarbonyl), alkyl (e.g. C₁₋₆-alkyl), alkynyl (e.g. C₁₋₆-alkynyl), cyano, haloalkoxy (e.g. C₁₋₆-haloalkoxy), haloalkyl (e.g. C₁₋₆-haloalkyl), halogen, hydroxy, hydroxyalkyl (e.g. C₁₋₆-hydroxyalkyl), nitro, NR_{A}R_{B}, and (NR_{A}R_{B})carbonyl;
A is a nonaromatic 4, 5, 6, 7, or 8-membered ring that contains 1 or 2 nitrogen atoms and, optionally, one sulfur or oxygen atom, wherein the nonaromatic ring is optionally substituted with 1, 2, or 3 substituents selected from the group consisting of alkenyl (e.g. C₁₋₆-alkenyl), alkoxy (e.g. C₁₋₆-alkoxy), alkoxyalkyl (e.g. C₁₋₆-alkoxy-C₁₋₆-alkyl), alkoxycarbonyl (e.g. C₁₋₆-alkoxycarbonyl), alkoxycarbonylalkyl (e.g. C₁₋₆-alkoxycarbonyl-C₁₋₆-alkyl), alkyl (e.g. C₁₋₆-alkyl), alkynyl (e.g. C₁₋₆-alkynyl), aryl, arylalkyl (e.g. aryl-C₁₋₆-alkyl), cycloalkyl (e.g. C₃₋₈-cycloalkyl), cycloalkylalkyl (e.g. C₃₋₈-cycloalkyl-C₁₋₆-alkyl), cyano, haloalkoxy (e.g. C₁₋₆-haloalkoxy), haloalkyl (e.g. C₁₋₆-haloalkyl), halogen, heterocycle, heterocyclealkyl (e.g. heterocycle-C₁₋₆-alkyl), heteroaryl, heteroarylalkyl (e.g. heteroaryl-C₁₋₆-alkyl), hydroxy, hydroxyalkyl (e.g. C₁₋₆-hydroxyalkyl), nitro, NR_{C}R_{D}, (NR_{C}R_{D})alkyl (e.g. (NR_{C}R_{D})-C₁₋₆-alkyl), (NR_{C}R_{D})carbonyl, (NR_{C}R_{D})carbonylalkyl (e.g. (NR_{C}R_{D})carbonyl-C₁₋₆-alkyl), and (NR_{C}R_{D})sulfonyl; and R_{A}, R_{B}, R_{C}, and R_{D} are independently selected from the group consisting of hydrogen, alkyl (e.g. C₁₋₆-alkyl), and alkycarbonyl (e.g C₁₋₆-alkylcarbonyl).
(iv) that inhibits PARPI, PARP2 and PARP3 is a compound of formula (VI)

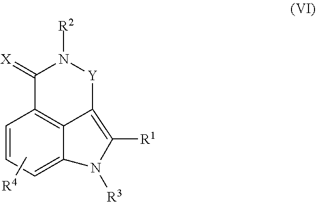

and isomers, salts, solvates, chemically protected forms, and prodrugs thereof wherein:
R₁ is: H; halogen; cyano; an optionally substituted alkyl (e.g. C₁₋₆-alkyl), alkenyl (e.g. C₂₋₆-alkenyl), alkynyl (e.g. C₂₋₆-alkynyl), cycloalkyl (e.g. C₃₋₈-cycloalkyl), heterocycloalkyl (e.g. C₂₋₈-heterocycloalkyl), aryl, or heteroaryl group (e.g., unsubstituted or substituted with one or more substituents selected from halogen, hydroxy, nitro, and amino, alkoxy (e.g. C₁₋₆-alkoxy), alkyl (e.g. C₁₋₆-alkyl), and aryl groups unsubstituted or substituted with one or more substituents selected from halogen, hydroxy, nitro, carboxy, and optionally substituted amino and ether groups (such as O-aryl)); or —C(O)—R₁₀, where R₁₀ is: H; an optionally substituted alkyl (e.g. C₁₋₆-alkyl), alkenyl (e.g. C₁₋₆-alkenyl), alkynyl (e.g. C₁₋₆-alkynyl), cycloalkyl (e.g. C₃₋₈-cycloalkyl), heterocycloalkyl (e.g. C₂₋₈-heterocycloalkyl), aryl, or heteroaryl group (e.g., unsubstituted or substituted with one or more substituents selected from halogen, hydroxy, nitro, amino, and alkyl (e.g. C₁₋₆-alkyl) and aryl groups unsubstituted or substituted with one or more substituents selected from halo, hydroxy, nitro, and amino); or OR₁₀₀ or NR₁₀₀R₁₁₀, where R₁₀₀ and R₁₁₀ are each independently H or an optionally substituted alkyl (e.g. C₁₋₆-alkyl), alkenyl (e.g. C₂₋₆-alkenyl), alkynyl (e.g. C₂₋₆-alkynyl), cycloalkyl (e.g. C₃₋₈-cycloalkyl), heterocycloalkyl (e.g. C₂₋₆-heterocycloalkyl), aryl, or heteroaryl group (e.g., unsubstituted or substituted with one or more substituents selected from alkyl (e.g. C₁₋₆-alkyl), alkenyl (e.g. C₂₋₆-alkenyl), alkynyl (e.g. C₂₋₆-alkynyl), cycloalkyl (e.g. C₃₋₈-cycloalkyl), heterocycloalkyl (e.g. C₂₋₈-heterocycloalkyl), aryl, and heteroaryl groups unsubstituted or substituted with one or more substituents selected from halogen, hydroxy, nitro, amino, and alkyl (e.g. C₁₋₆-alkyl) and aryl groups unsubstituted or substituted with one or more substituents selected from halogen, hydroxy, nitro, and optionally substituted amino groups);
R₂ is H or alkyl (e.g. C₁₋₆-alkyl);
R₃ is H or alkyl (e.g. C₁₋₆-alkyl);
R₄ is H, halogen or alkyl (e.g. C₁₋₆-alkyl);
X is O or S;
Y is (CR₅R₆)(CR₇R₈)ₙ or N—C(R₅), where:
n is 0 or 1;
R₅ and R₆ are each independently H or an optionally substituted alkyl (e.g. C₁₋₆-alkyl), alkenyl (e.g. C₂₋₆-alkenyl), alkynyl (e.g. C₂₋₆-alkynyl), cycloalkyl (e.g. C₃₋₈-cycloalkyl), heterocycloalkyl (e.g. C₂₋₈-heterocycloalkyl), aryl, or heteroaryl group (e.g., unsubstituted or substituted with one or more substituents selected from halogen, hydroxy, nitro, amino, and lower alkyl (e.g. C₁₋₄-alkyl), lower alkoxy (e.g. C₁₋₄-alkoxy), or aryl groups unsubstituted or substituted with one or more substituents selected from halogen, hydroxy, nitro, and amino); and
R₇ and R₈ are each independently H or an optionally substituted alkyl (e.g. C₁₋₆-alkyl), alkenyl (e.g. C₂₋₆-alkenyl), alkynyl (e.g. C₂₋₆-alkynyl), cycloalkyl (e.g. C₃₋₈-cycloalkyl), heterocycloalkyl (e.g. C₂₋₈-heterocycloalkyl), aryl, or heteroaryl group (e.g., unsubstituted or substituted with one or more substituents selected from halogen, hydroxy, nitro, amino, and lower alkyl (e.g. C₁₋₄-alkyl), lower alkoxy (e.g. C₁₋₄-alkoxy), and aryl groups unsubstituted or substituted with one or more substituents selected from halogen, hydroxy, nitro, and amino);
where when R₁, R₄, R₅, R₆, and R₇ are each H, R₈ is not unsubstituted phenyl.

In a preferred embodiment of the ALC1i or the bifunctional compound for use of the fourth aspect of the invention, or the PARPi for use of the fifth aspect of the invention the PARPi is selected from the group consisting of Olaparib, Talazoparib, Niraparib, Rucaparib, and Veliparib, in particular of Veliparib, Olaparib, and Talazoparib.

In a preferred embodiment the ALC1i or the bifunctional compound for use of fourth aspect of the invention or the PARPi for use of the fifth aspect of the invention the ALC1i is the inhibitor of ALC1 of any of the first or further aspect of the invention or the bifunctional compound of the second aspect.

In a preferred embodiment the ALC1i or the bifunctional compound for use of the fourth aspect of the invention or the PARPi for use of the fifth aspect of the invention the tumor disease is selected from hepato cellular carcinoma, breast cancer, ovarian cancer, prostate cancer, and colorectal cancer.

In a preferred embodiment the ALC1i or the bifunctional compound for use of the fourth aspect of the invention or the PARPi for use of the fifth aspect of the invention (i) the ALC1i potentiates the cancer-cell killing efficacy of the PARPi, (ii) a reduced amount of PARPi is administered, and/or (iii) PARPi resistance is bypassed.

In a preferred embodiment the ALC1i for use of the fourth aspect of the invention or the PARPi for use of the fifth aspect of the invention the PARPi and ALC1i are administered concomitantly or separately.

In a sixth aspect the present invention relates to a kit of parts comprising separately packaged a PARPi and an ALC1i or a composition comprising a PARPi and an ALC1i, preferably with instructions for use to treat or ameliorate a proliferative disease.

BRCA1 and BRCA2 proteins are involved in both promoting homologous recombination (HR)-mediated DNA repair and also controlling the stability of stalled replication forks. Many tumor types, including, e.g., breast cancer, ovarian cancer, prostate cancer, pancreatic carcinomas, fallopian tube cancer, peritoneal cancer, acute myeloid leukemia, and uveal melanoma often have underlying defects in BRCA1 or BRCA2 activity. These defects are often due to germline or somatic mutations in the BRCA1 or BRCA2 genes. These tumors, with underlying defects in HR repair, are typically sensitive to PARP inhibitors.

However, they can develop PARP inhibitor resistance over the time. To treat such tumors the use of the ALC1i for use of the first aspect of the invention the bifunctional compound of the second aspect of the invention, the pharmaceutical of the third aspect of the invention, the ALC1i or the bifunctional compound for use of the fourth aspect of the invention is particular suitable if used alone or in combination with PARPi. In a preferred embodiment of the ALC1i for use according to the first aspect of the invention the bifunctional compound of the second aspect of the invention, the pharmaceutical of the third aspect of the invention, the ALC1i or the bifunctional compound for use of the fourth aspect of the invention or the PARPi for use of the fifth aspect of the invention the proliferative disease is selected from a cancer.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof. In some embodiments, the cancer is breast cancer, ovarian cancer, prostate cancer, pancreatic carcinomas, colorectal cancer, hepatocellular carcinoma, uterine cancer, bone cancer (preferably osteosarcoma), gastric cancer, gastroesophageal cancer, non-small cell lung cancer, fallopian tube cancer, peritoneal cancer, acute myeloid leukemia, or uveal melanoma. In some embodiments, the cancer is basal-like cancer, basal-like breast cancer, triple negative breast cancer, high grade cancer, or high grade serous ovarian cancer cell.

Thus, a cancer that is BRCA1 deficient or BRCA2 deficient refers to a cancer that has one or more cells having abnormal BRCA1 levels or activities, or abnormal BRCA2 levels or activities. These abnormal levels or activities interfere with the normal function of BRCA1 or BRCA2, and can cause a defect in HR-mediated DNA repair or decrease the stability of replication forks.. Preferably the proliferative disease is selected from a BRCA-1 and/or BRCA-2-deficient tumor, and/or the proliferative disease is selected from hepatocellular carcinoma, breast cancer, ovarian cancer, prostate cancer, colorectal cancer, gastric cancer, gastroesophageal cancer, non-small cell lung cancer, or pancreatic cancer.

Thus in a seventh aspect the ALC1i for use according to the first aspect of the invention the bifunctional compound of the second aspect of the invention, the pharmaceutical of the third aspect of the invention, the ALC1i or the bifunctional compound for use of the fourth aspect of the invention or the PARPi for use of the fifth aspect of the invention the proliferative disease is selected from a cancer or the kit of the sixth aspect of the invention, wherein the proliferative disease, preferably cancer, and more preferably the cancer is selected from a BRCA1 and/or 2-deficient tumor, and a tumor in which expression of PARPi, PARP2, PARP3 and/or ALC1 is increased in comparison to non-tumor cells.

Preferred examples of cancers treatable according to the various aspects of the invention are ovarian and fallopian tube cancer, breast cancer, pancreatic and gastric cancer, colorectal cancer and pulmonary cancer.

Preferably, the cancer to be treated in accordance with the various aspects of the invention has relapsed or progressed, preferably after a first line chemotherapy. Thus, preferably, the inhibitor of ALC1 (ALC1i) according to the present invention is for use as second line or third line therapy.

Preferably, the cancer to be treated is at stage III (locally advanced) or IV (metastatic).

Preferably, the cancer to be treated in accordance with the various aspects of the invention has underlying defects in DNA damage repair, like HR repair, i.e. is a HR deficient cancer. In one embodiment, the cancer cells have mutations/deletions/insertions in one or more DNA repair genes as listed in Tables 13 and 14.

In one embodiment, the cancer to be treated or the cancer patients to be treated are selected based on the presence of tumor markers. In the clinical setting cancer/patient selection would include but is not limited to eligible tumor biomarkers, i.e., deleterious mutations including BRCA1, BRCA2, BARD1, BRIP1, ANCA, FANCE, NBN, PALB2, RAD51C, RAD51D, RAD51 and/or RAD51B and/or other gene variants in the HR pathway as listed in Table 13 and Table 14.

Preferably, the patient to be treated in accordance with the various aspects of the invention is a cancer patient, preferably having breast cancer, ovarian cancer, prostate cancer, pancreatic carcinomas, gastric, gastroesophageal, non-small cell lung cancer, colorectal cancer, hepatocellular carcinoma, uterine cancer, bone cancer (preferably osteosarcoma), fallopian tube cancer, peritoneal cancer, acute myeloid leukemia, or uveal melanoma, and optionally having mutations/deletions/insertions in one or more of the HR genes as listed in Tables 13 and 14.

### Experimental Section

### Cell Lines Used

As a BRCA wild-type cell line, MDA-MB-231 cells were used. The cells were established from an aneuploid female human. The cells were extracted from the mammary gland (breast) in the metastatic site as a pleural effusion. (MDA-MB-231 (ATCC® HTB-26™ Homo sapiens epithelial mammary gland) It is available from numerous sources including ATCC® HTB-26™

As a BRCA negative cell line, SUM-149-PT cells were used. The cell line is a triple negative breast cancer (TNBC) cell line, derived from primary human invasive ductual carcinoma metastatic nodule from a 40 year old female. It contains a hemizygous BRCA1 mutation (p.Pro724Leufs*12) and is available from numerous sources including bioIVT.

As an example for pancreatic cancer, PSN1 cells were used. The human cell line was derived from pancreatic adenocarcinoma tissue. It harbors an amplification of c-myc and activated c-Ki-ras and a loss of one of the two p53 alleles. The cell line is available from numerous sources including MERCK (94060601).

BxPC-3 cells were used. The cells were extracted from the pancreas tissue of a 61-year-old female with adenocarcinoma. The established cell line does not express CFTR (cystic fibrosis transmembrane conductance regulator), but it expresses mucin, the pancreas cancer specific antigen, and CEA (carcinoembryonic antigen). The cells are available from numerous sources including MERCK (93120816).

CaCo2 cells were used. The cells are established from colon tissue from a 72-year old female with colorectal adenocarcinoma and is available at ATCC (Caco-2 [Caco2] HTB-37™)

Capan-1 cells were used. These cells were isolated from the pancreas of a 40 year old male with pancreatic adenocarcinoma. The cells are available at ATCC (Capan-1 HTB-79™)

22Rv1 cells were used. The cell line is derived from a xenograft that was serially propagated in mice after castration-induced regression and relapse of the parental CWR22 xenograft that was androgen-dependent. The cells originate from prostate carcinoma and are epithelial. They express PSA (prostate-specific antigen) and have a damaging mutation in BRCA2. The cell line is available at numerous sources including Accegen (ABC-TCO0004).

DU145 cells were used. The cells were isolated from the brain of a 69-year-old male with prostate cancer, the established cell line has epithelial morphology. It is hypotriploid and has mutations in BRCA1, BRCA2, RAD50, WRN and TP53. The cell line is available at ATCC HTB-81™

DLD1-wt cells were used. The cells were extracted from the large intestine of an adult male patient with colorectal carcinoma and a cell line was established. They are pseudodiploid(2n=46), express p53 antigen and have damaging mutations in BRCA2, FANCA, and TP53. The cell line is available at numerous sources including MERCK (90102540).

DLD1-BRCA2−/− cells were used. This cell line originated from the DLD1-wt and is a BRCA2 knockout cell line. It is available at several sources including Creative Biogene (CSC-RT0028).

HCT116 cells were used. This cell line originates from colon tissue from an adult male with colorectal carcinoma. It has damaging mutations in BRCA2, FANCA and POLD1. This cell line is available at numerous sources including the DSMZ (ACC 581).

PC-3 cells were used. The cell line originates from a 62-year-old male patient with a grade IV prostatic adenocarcinoma, specifically from a bone metastasis. The cells are epithelial, near triploid and have a damaging mutation in TP53. They are available at numerous sources including MERCK (90112714).

SW620 cells were used. The cells were isolated from the lymph node of a 51-year-old male with Dukes' C colorectal adenocarcinoma and a cell line was established. The cell line is available at numerous sources including ATCC CCL-227™

T47D cells were used. The cell line was established from a 54-year-old female human with an infiltrating ductal carcinoma of the breast. The cells were extracted from the mammary gland (breast) as a pleural effusion. They are available at numerous sources including ATCC HTB-133™

HUH7 cells were used. They are available for example at the cell lines service (CLS.shop). these cells were isolated from a the liver tumor of a 57-year old male.

HCC1428 cells were used. They are available at ATCC (HCC1428 CRL-2327™) and were derived from the mammary gland of a white female patient with adenocarcinoma.

HCC1937 cells were used. They were isolated from a 23-year-old female with a primary ductal carcinoma, specifically from a mammary gland. The cell line has a homozygous BRCA1 mutation and is negative for expression of p53 and Her2-neu, the morphology is epithelial. The cells are available at several sources including Amsbio (Cat. No. AMS.EP-CL-0093).

MDA-MB-436 cells were used. The cell line is derived from a 43-year-old female with breast adenocarcinoma, it was extracted from the mammary gland in the metastatic site as a pleural effusion. The cells are pleomorphic with multinucleated component cells. They are available at numerous sources including Accegen (Cat. No. ABC-TC0655).

HEPG2 cells were used. The cell line was established from a 15-year-old male patient with a hepatocellular carcinoma. The cells express several major plasma proteins like albumin, fibrinogen, and alpha 2-macroglobulin, the morphology is epithelial-like. The cell line is available at ATCC HB-8065™

LnCap cells were used. The cells were isolated from the left supraclavicular lymph node of a male with metastatic prostate carcinoma and are available at ATCC (LNCaP clone FGC CRL-1740™)

U2OS cells were used. They are available at ATCC (U-2 OS HTB-96™) and were isolated from the tibia of a 15 year old female with osteosarcoma.

Synergy with PARPi

For determination of the ZIP synergy score, a 2-D titration of two compounds is added to cells in the 96h-SRB-survival assay format. The score is calculated by adding the readout of the survival data from the SRB assay of at least 3 replicate plates to an open-source program called Synergy Finder.

“SynergyFinder (https://synergyfinder.fimm.fi) is a stand-alone web-application for interactive analysis and visualization of drug combination screening data. Since its first release in 2017, SynergyFinder has become a widely used web-tool both for the discovery of novel synergistic drug combinations in pre-clinical model systems (e.g. cell lines or primary patient-derived cells), and for better understanding of mechanisms of combination treatment efficacy or resistance” (Ianevski et al. 2020).

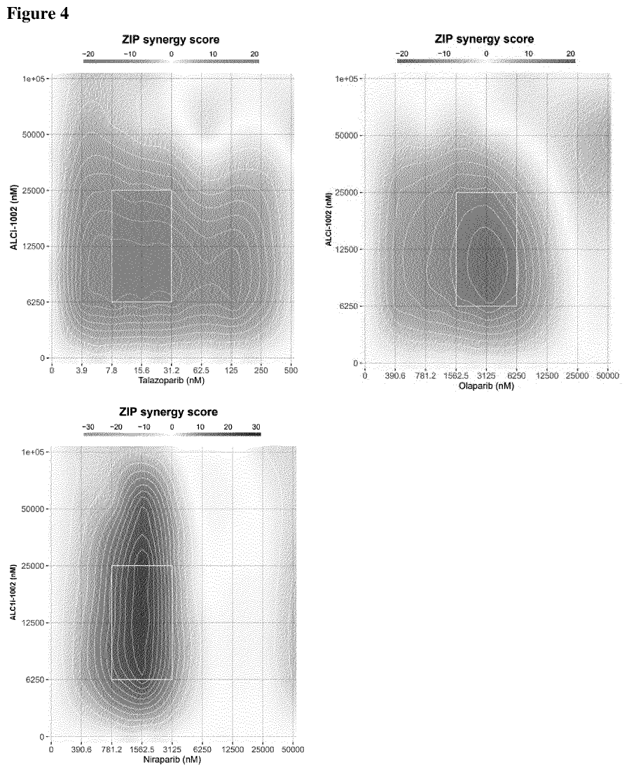

In the Zero interaction potency (ZIP) model, the drug interaction relationship is determined by comparison of the change in potency of the dose-dependent curves between individual drugs and their combination (https://synergyfinder.fimm.fi/synergy/synfin_docs/). The model is further described in https://www.ncbi.nlm.nih.gov/pmc/articles/PMC4759128/. Results for treatment with ALC1 inhibitors and PARP inhibitors together are shown in FIG. 4.

Inhibitor Vs. Response (Cell Survival) Assay

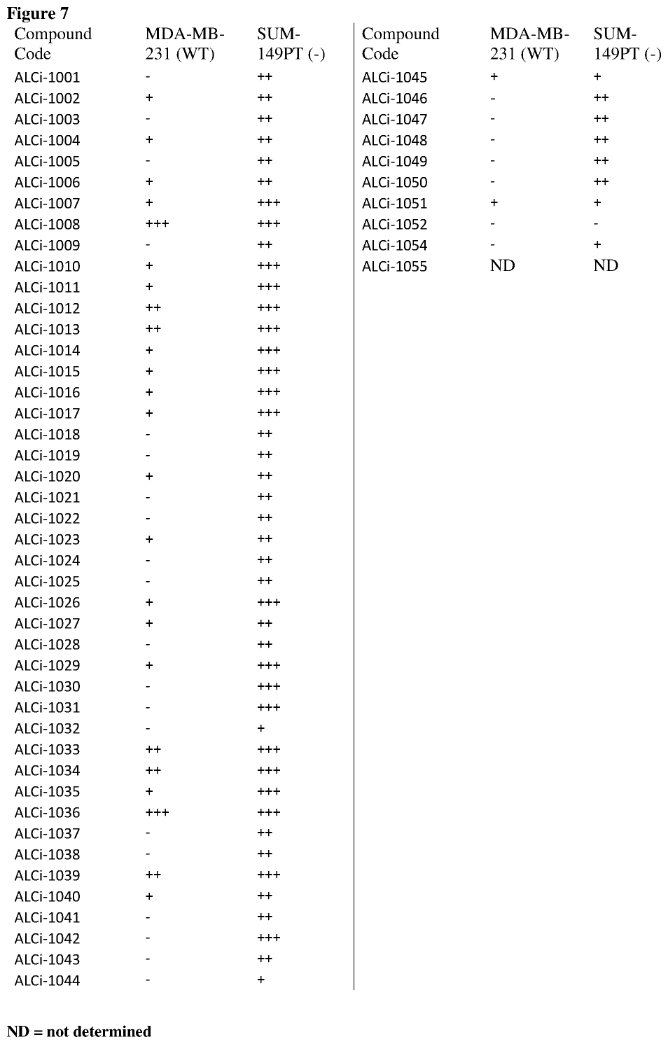

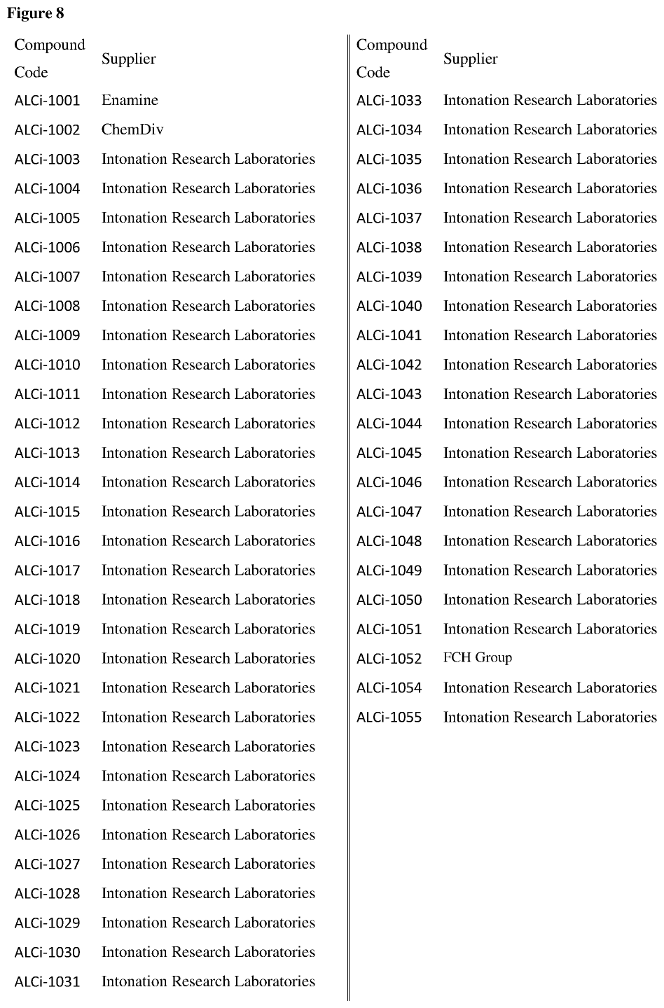

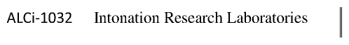

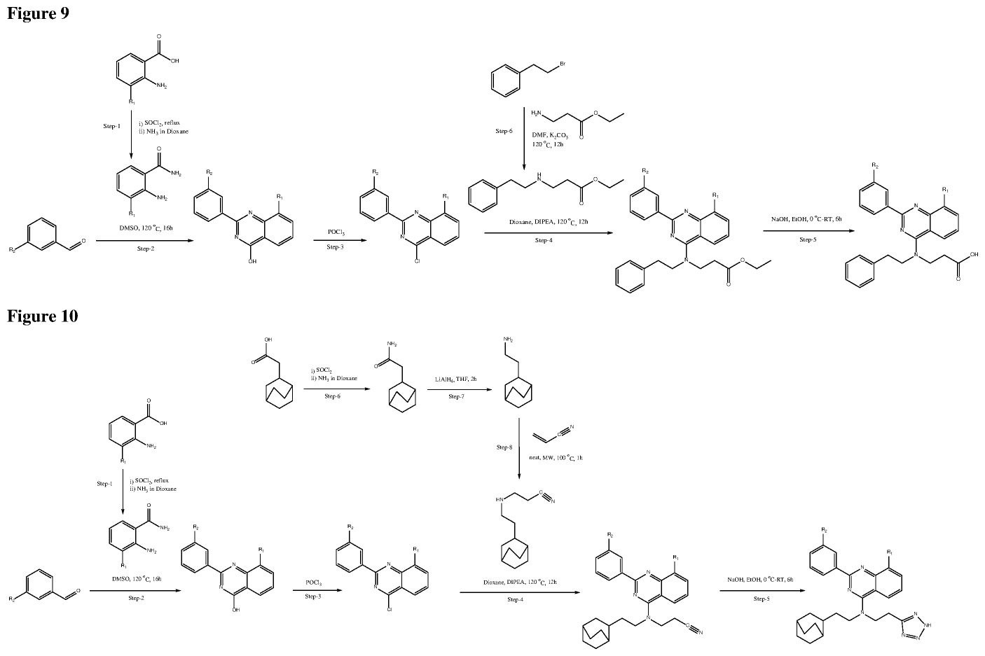

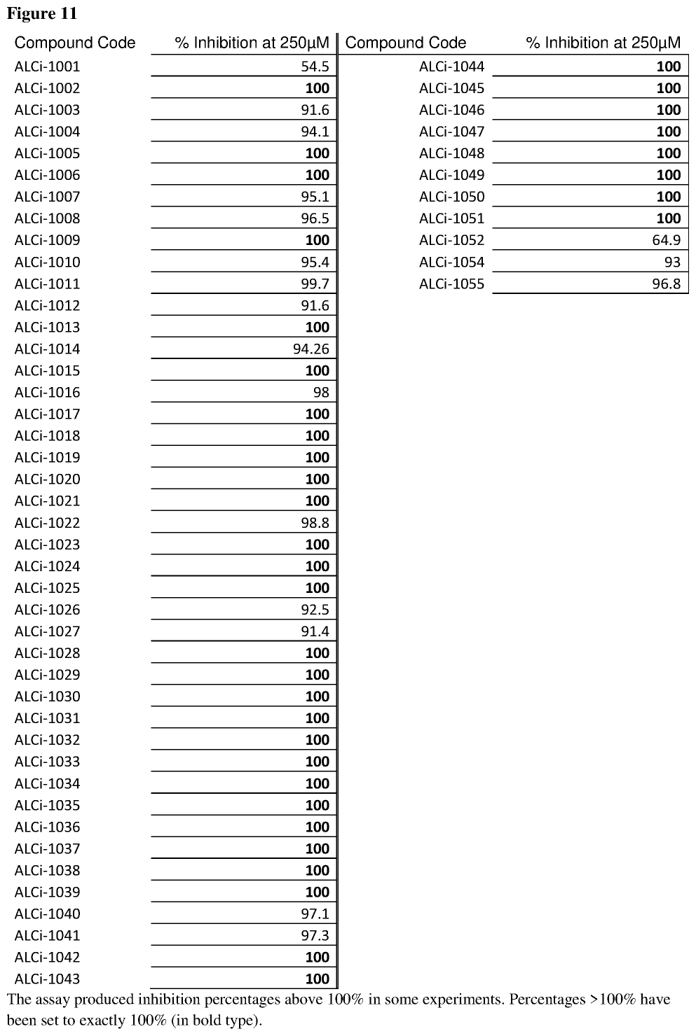

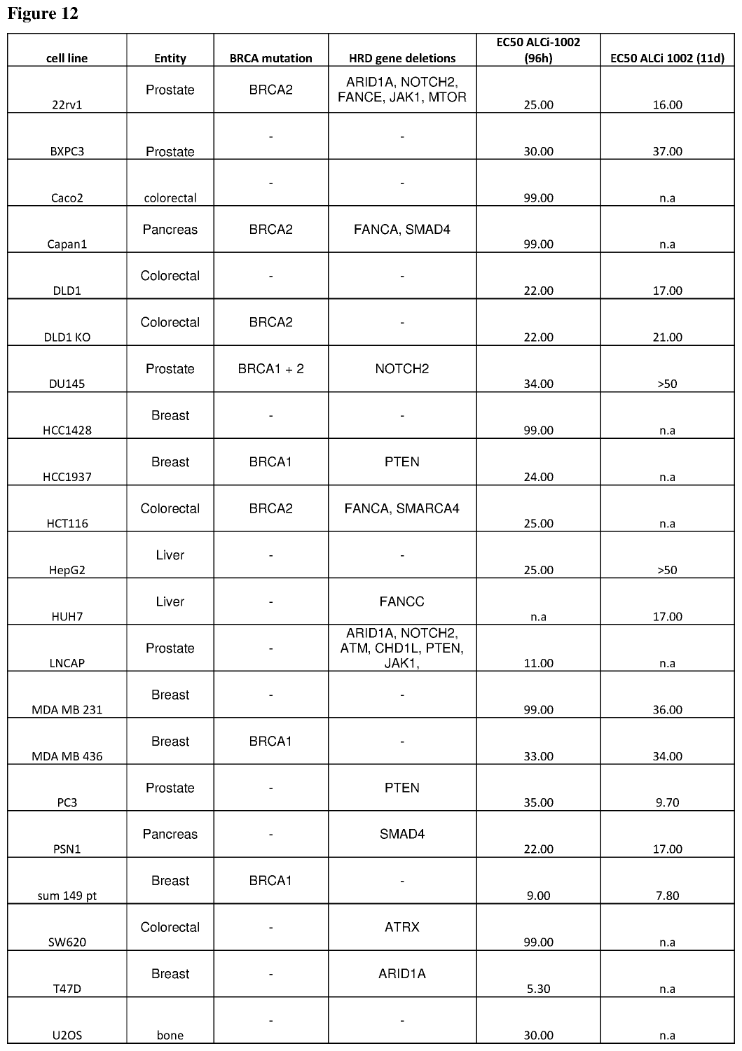

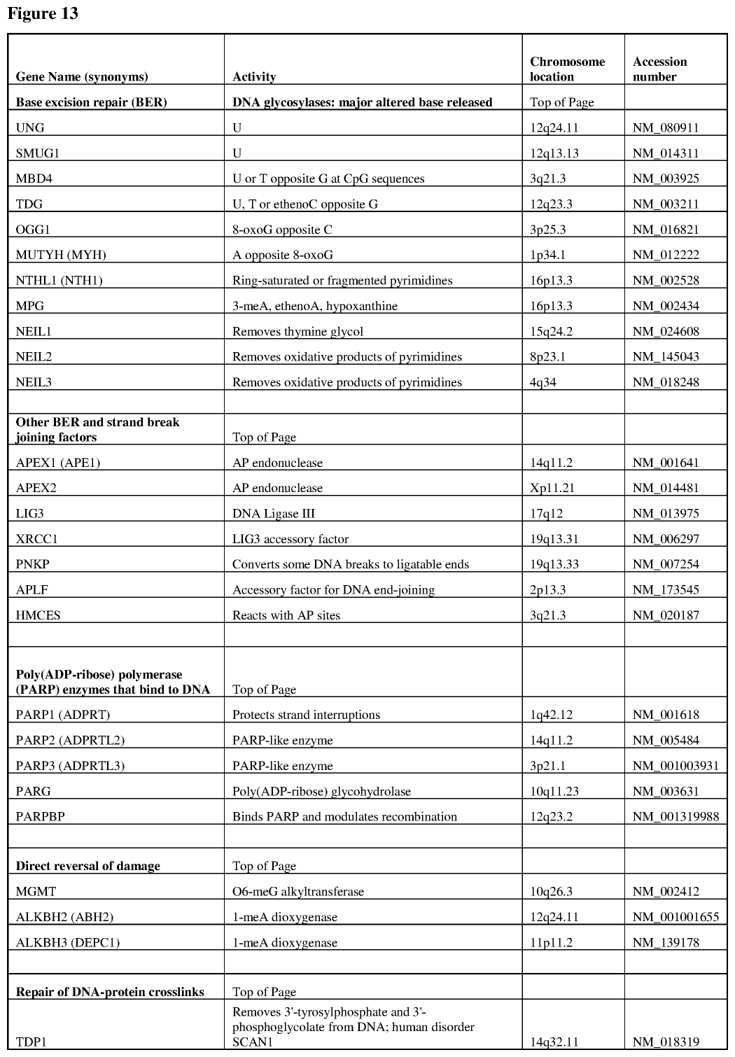

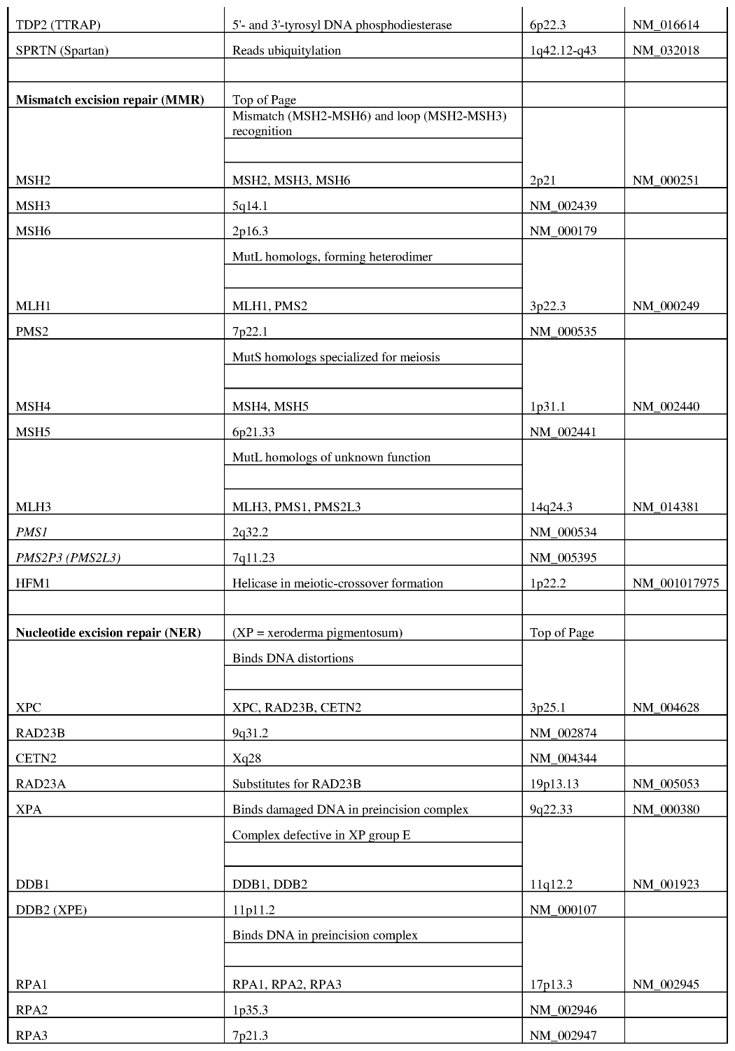

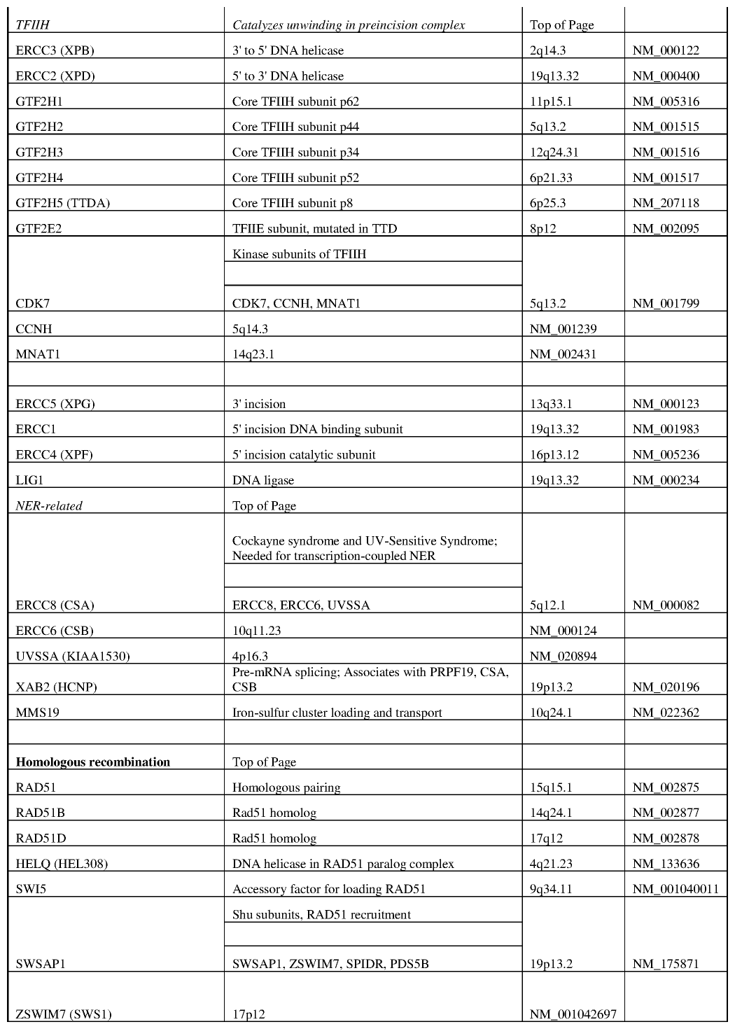

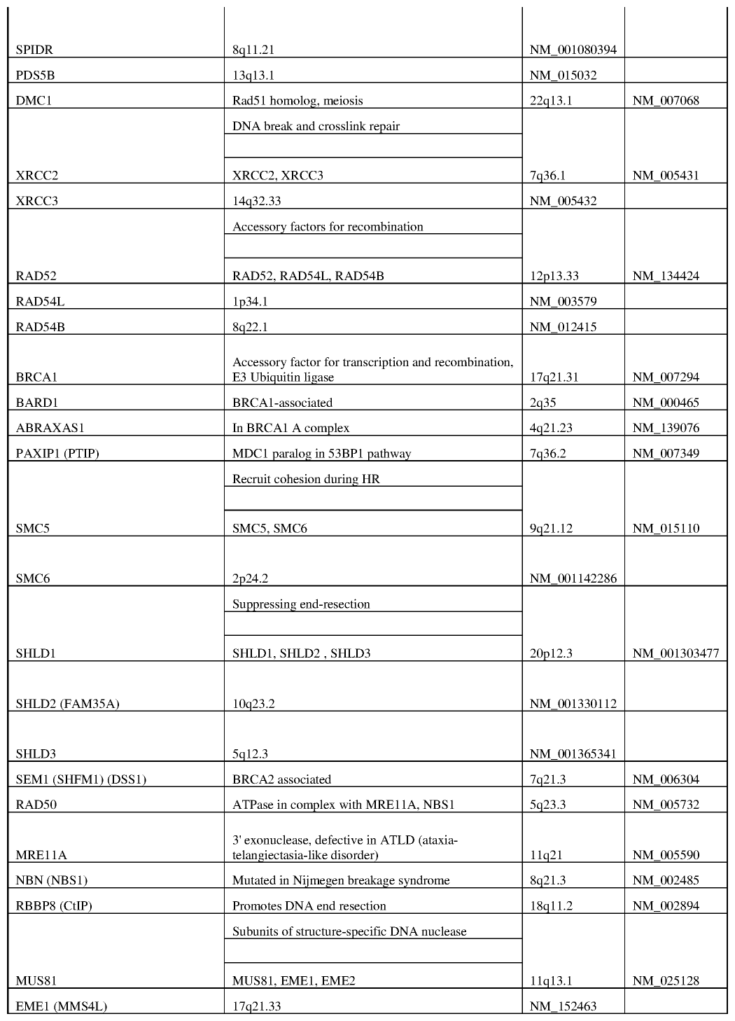

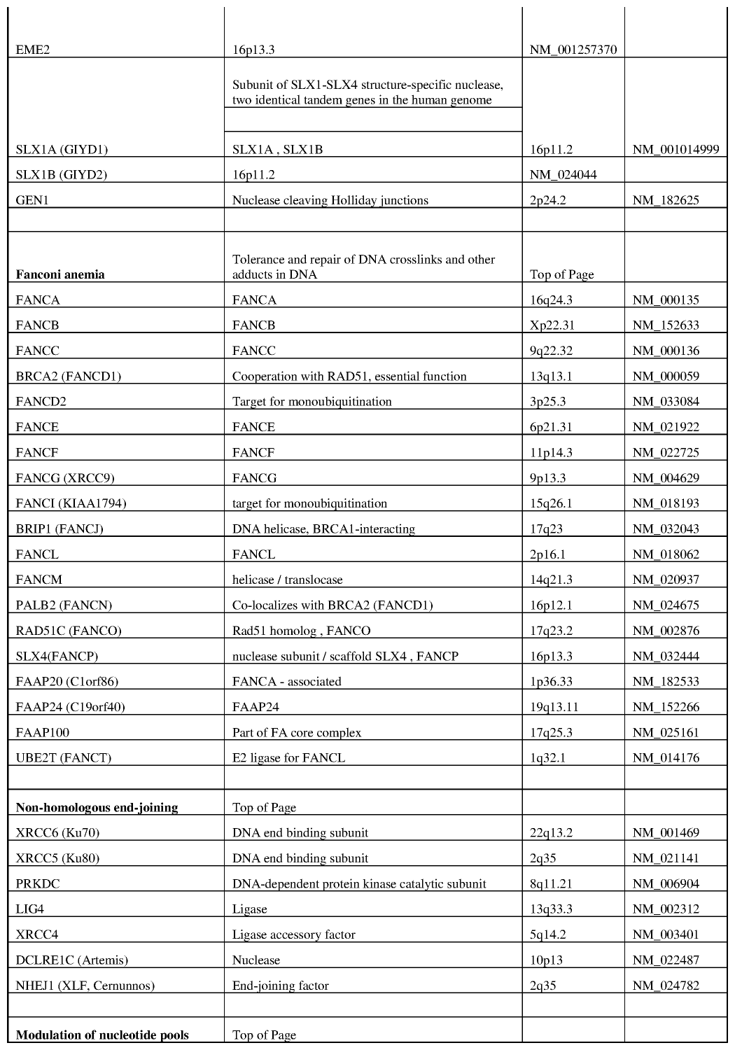

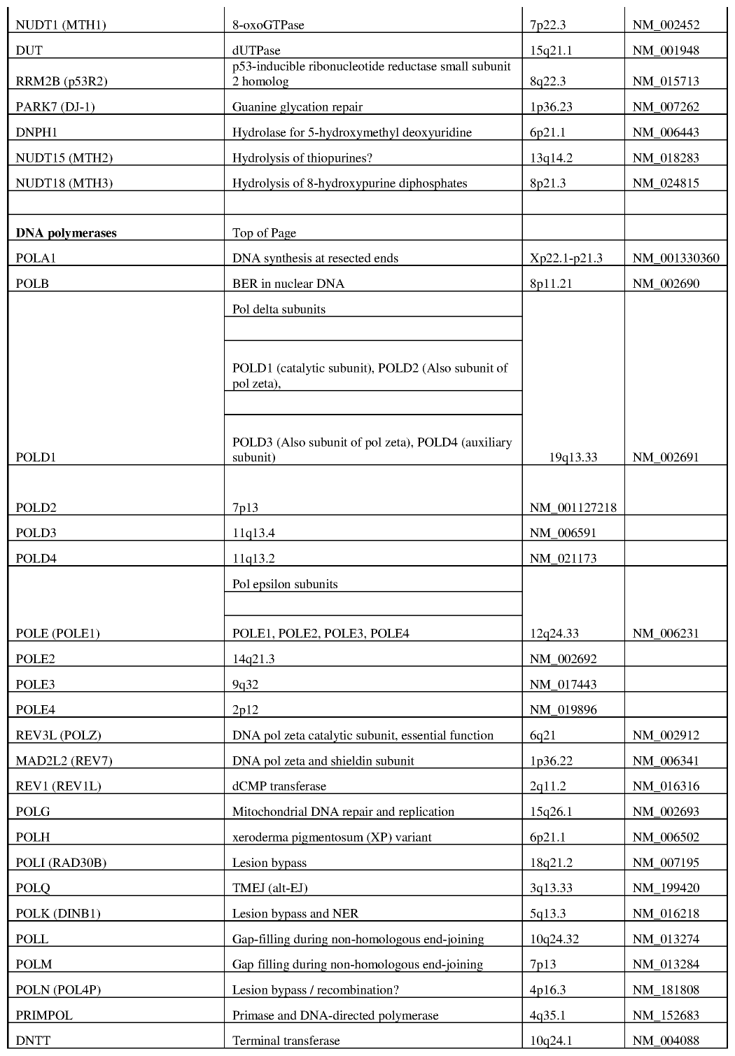

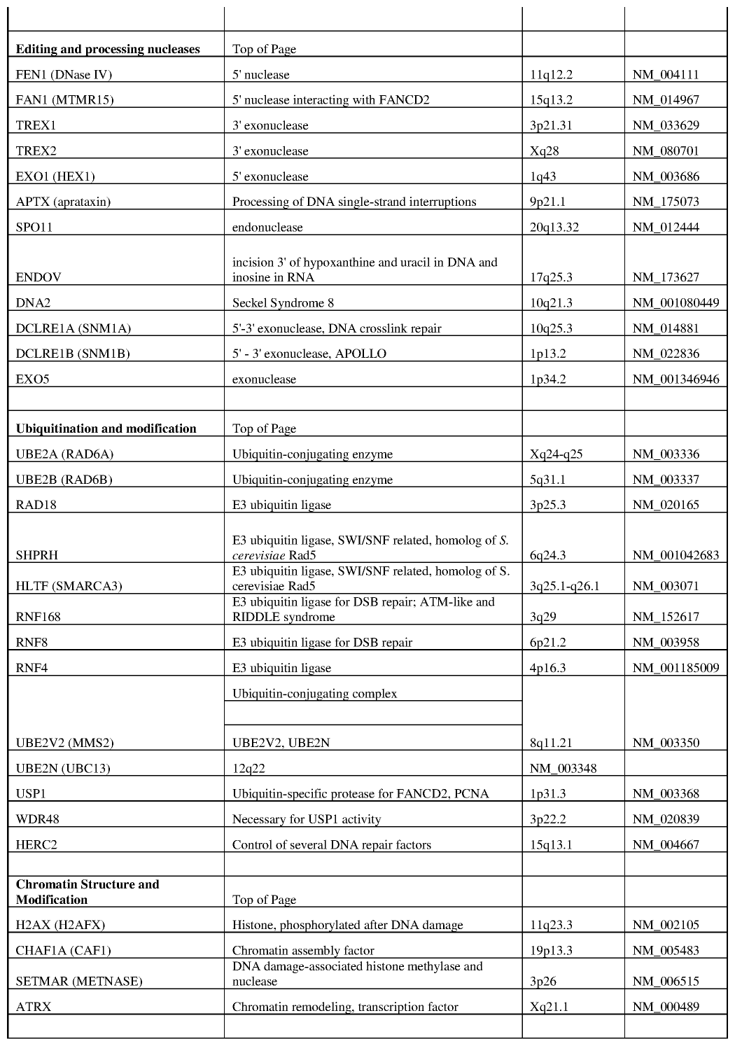

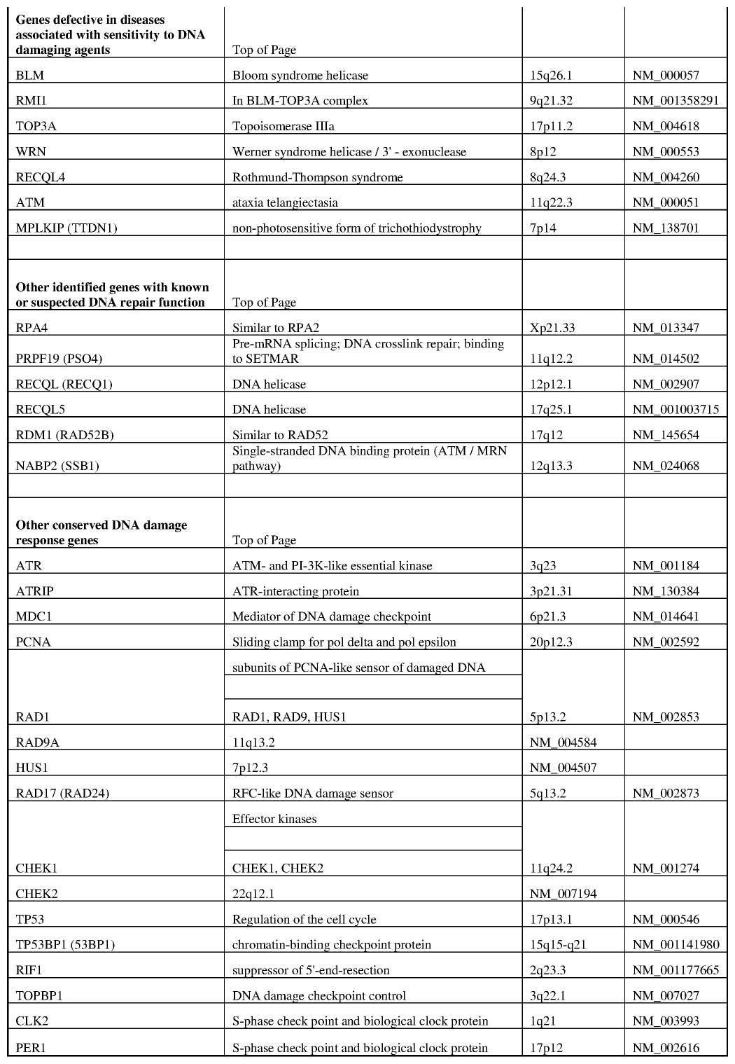

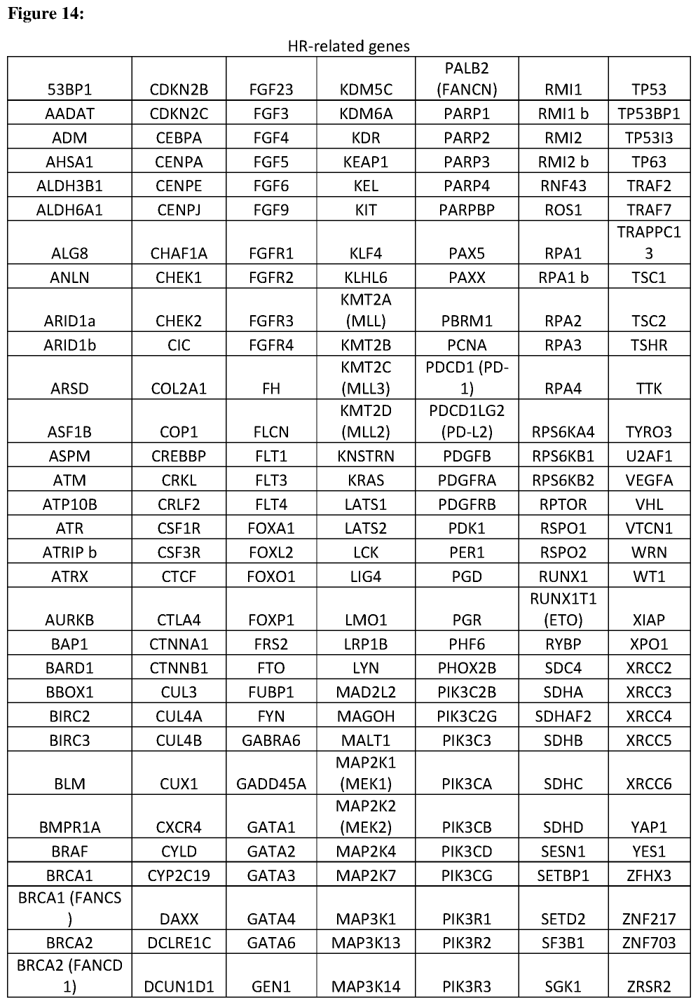

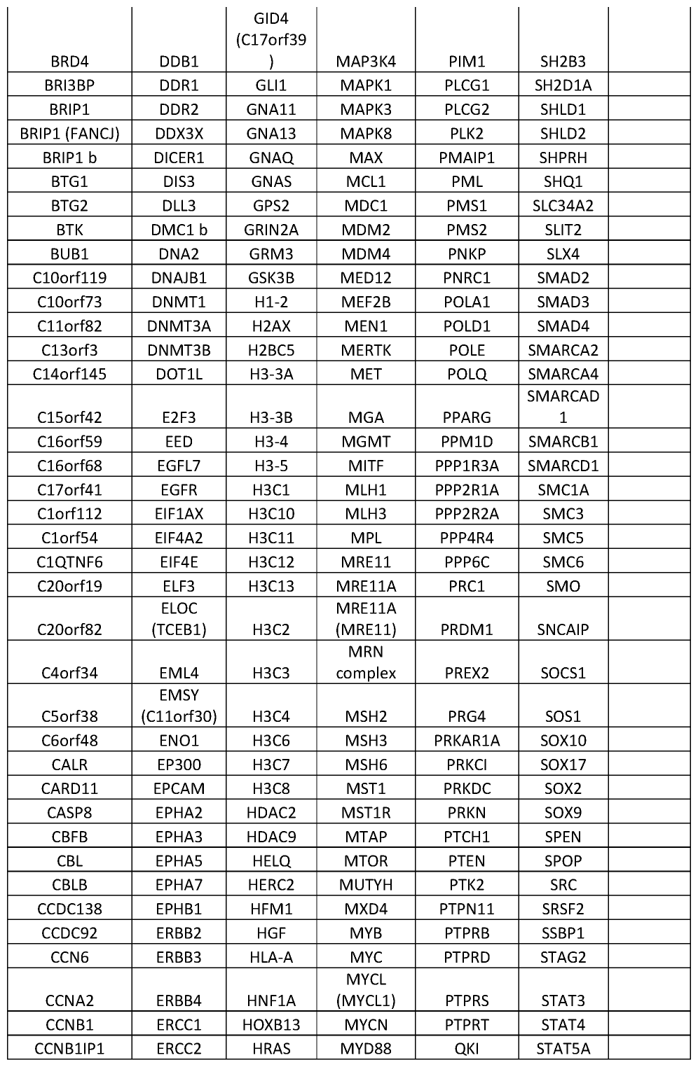

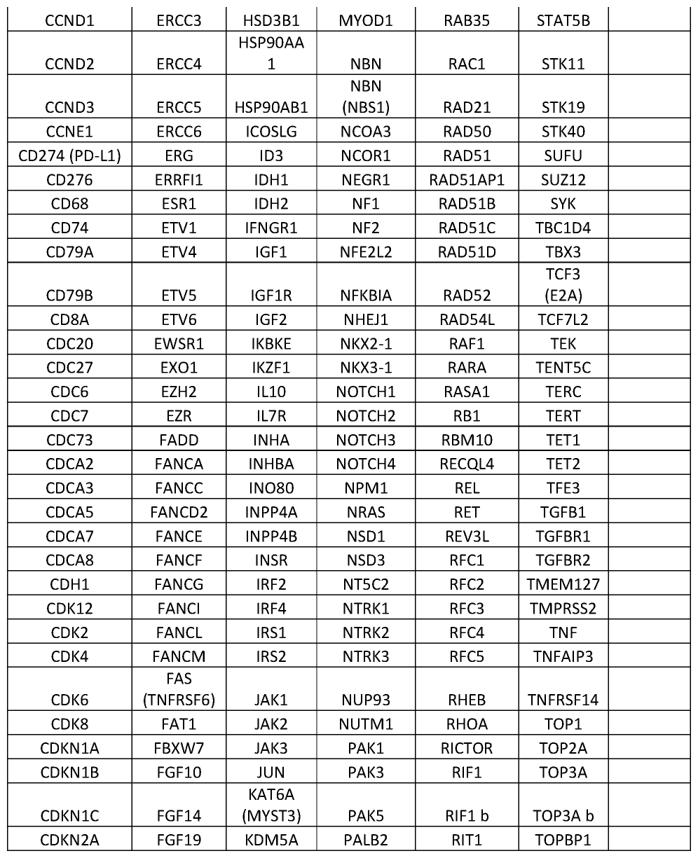

For validation of the small molecule inhibitors, the synthetic lethality of BRCA and ALC1 was addressed using MDA-MB-231 cells as a BRCA wild-type cell line and SUM-149-PT as a BRCA1 deficient cell line. Cells were seeded in 96-well plates (5000 cells/well) and treated with titrations of ALC1 inhibitors starting at 50 μM. As a control for “no-treatment”, DMSO was added to the cells. The cells were cultured at 37° C., CO₂ 5% for 5 days until they were fixed with 10% TCA for 1 h and stained with sulforhodamine dye for 30 minutes. After washing the cells with 1% Acetic Acid, 10 mM Tris (pH 10.5) solution was used to solubilize the stained cells. The absorbance was measured at 492 nm using the SUNRISE TECAN, the data were normalized to the number of cells at timepoint 0 and to 100% survival (=DMSO control) and analyzed using GraphPad Prism. Survival curves were fitted using “Inhibitor vs. response curves with variable slope (four parameters)”. The IC₅₀ values for treatment with ALC1 inhibitors are shown in FIG. 7.

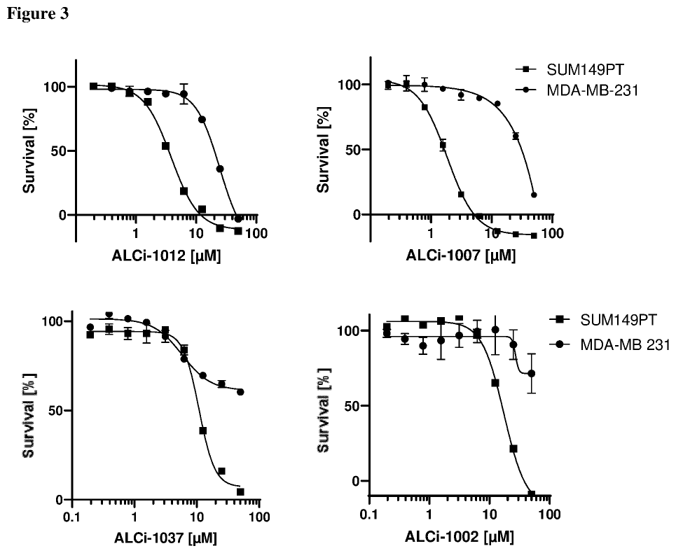

For further validation of the compounds, colony formation assays were applied. Here, less cells were seeded for the assay (100 cells/well). The cells were treated with ALC1 inhibitor or with a combination of PARPi and an ALC1 inhibitor for 11 days. The cells were fixed and data was analyzed as mentioned above. Results for treatment with ALC1 inhibitors are shown in FIGS. 3 and 7.

### Fret-Based Nucleosome Sliding Assay

This assay utilizes mid-positioned mononucleosomes that allow for monitoring the sliding activity of the ALC1 remodeling enzyme using a FRET readout. Each nucleosome is labeled with two FRET dyes: the octamer is labeled with Cy5 (Cy5-maleimide coupling to H2B) and one of the DNA ends is labeled with Cy3. The DNA template includes the 147 bp 601 DNA positioning sequence flanked by DNA overhangs on each side. Other nucleosome positioning sequences, both artificial constructs and naturally occurring sequences, even if less efficient than the 601 sequence in positioning nucleosomes, can also be used. The nucleosomes are assembled by salt gradient dialysis using purified, Cy5-labeled histone octamers and purified, Cy3-labeled DNA templates to yield the FRET-labeled mid-positioned nucleosomes. These nucleosomes will start with low FRET and will have a low Cy5 fluorescence signal when excited with the Cy3 excitation maximum wavelength as the two fluorophores are too far apart for efficient FRET. As the ALC1 remodeling reaction proceeds and the remodeling enzymes slides the octamer towards the DNA end, the distance between the two FRET dyes decreases and the signal from Cy5/FRET increases. Hence, increase in FRET can be directly used as readout for sliding.

### DNA Template Construction for Mononucleosome Reconstitution

For reconstitution of mononucleosomes, the non-natural Widom 601 nucleosome positioning sequence was used as a high affinity binding site for the histone octamer (see Lowary, P. T. & Widom, J. New DNA sequence rules for high affinity binding to histone octamer and sequence-directed nucleosome positioning. J. Mol. Biol. 276, 19-42 (1998)). Cy3-labeled DNA containing these 601 sequences can be constructed using methods including PCR amplification, restriction digestion of DNA plasmids followed by a Klenow end labeling reaction or other standard molecular biological techniques.

### Preparation of ALC1 and Histone Octamers

Full length human ALC1 (1-897) or truncated versions thereof were expressed and purified as N-terminally 6× His-tagged fusion protein from E. coli as published before (Singh, H. R., et al., 2017).

Human histone proteins were recombinantly expressed in E. coli (either using classical IPTG induction or using autoinduction media) and purified from E. coli inclusion bodies. The purification scheme includes the extraction/solubilization of histones from inclusion bodies using guanidium chloride, followed by reverse phase chromatography. The purified histones were lyophilized resulting in TFA-salts of the purified histone proteins.

### Preparation of Nucleosomes

To assemble the nucleosomes, template DNA (250 g/ml final concentration) was mixed with purified histone octamers in a high salt buffer at different molar ratios of histone octamers to DNA. The ionic strength of this mixture was then reduced <600 mM NaCl by continuous dialysis against a low salt buffer at 4° C. Finally, the material was dialyzed against TEA-20 (10 mM triethanolamine-Cl pH 7.5, 20 mM NaCl, 0.1 mM EDTA). The best molar ratio, i.e. the ratio that yields full assembly of the DNA into nucleosomes was picked and used for further assemblies.

Alternatively, the nucleosomes can be assembled by other methods such as deposition of histone octamers onto DNA using polyglutamate or histone chaperones, or by salt step dilution.

### Method of Measuring ALC1 Mediated Sliding; ALC1 Nucleosomal Sliding Assay

Sliding reactions were performed in 384 well plates at RT in 10 mM Tris-HCl, pH 8.1, 75 mM KCl, 1 mM MgCl₂, 1.0 mM EGTA, 10% glycerol, 0.5 mM dithiothreitol (DTT), 0.01% TritonX100, 0.02% NP40 and reaction mixtures contained mid-positioned nucleosome, tri-ADP ribose or (ADP-ribose). and ALC1 chromatin remodeling enzyme. Sliding was initiated by addition of ATP followed by shaking the plates for 5 sec at 1450 rpm and sealing them with a foil compatible with fluorescence reading. The FRET signal was immediately recorded using a fluorometer (BMG reader PheraStar FSX, channel A: excitation 520 nm, emission 680 nm; channel B: excitation 520 nm, emission 590 nm) and unless stated otherwise, remodeling proceeded for 30 min.

The FRET signal was calculated as the signal at 680 nm (emission of Cy5) divided by the signal at 590 nm (emission of Cy3) and multiplied by 10,000. To obtain an apparent rate of ALC1-mediated nucleosome sliding, the increase in FRET as a function over time was plotted and the initial velocities of ALC1-mediated nucleosome sliding were obtained by fitting the resulting kinetic trace by a linear curve fit.

High Throughput Screening (HTS) and IC₅₀ Determination for Molecules that Modulate ALC1-Mediated Sliding

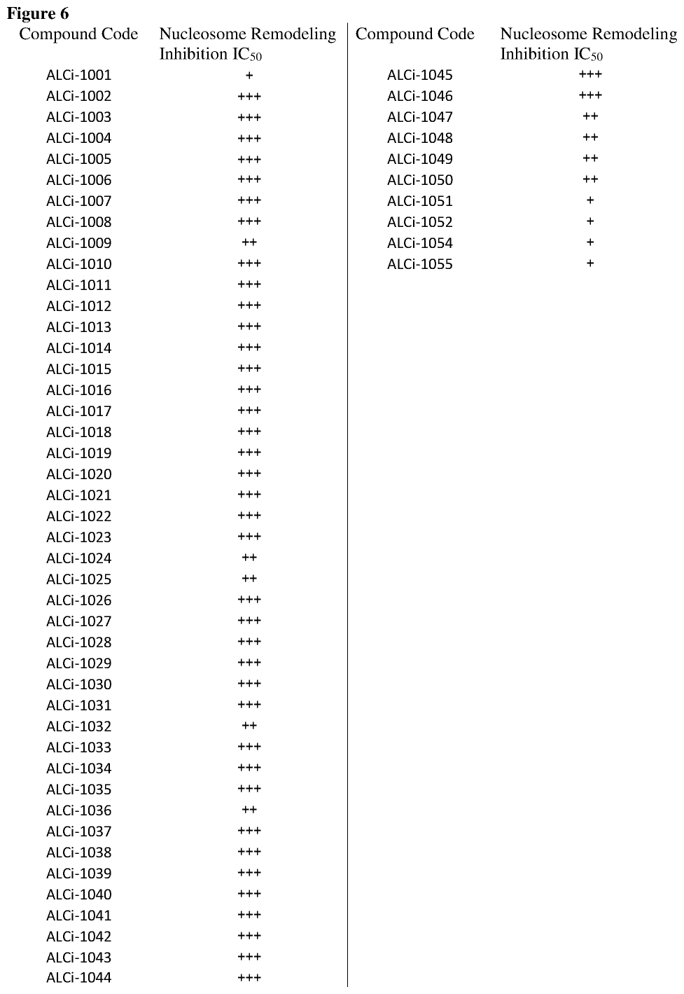

For High Throughput Screening (HTS) and IC₅₀ determination for compounds that modulate the sliding activity of ALC1, the nucleosome was incubated for 30 min as described above with ALC1 and triADP-ribose or (ADP-ribose)ₙ in the presence of the putative ALCi prior to initiating sliding by ATP addition. Then, the rate of sliding (initial velocity) was determined as described above and compared against the rate of sliding in the absence of the putative modulator/compound (% inhibition). For IC₅₀ determination, the observed % inhibition (y-axis) was plotted against compound concentrations (x-axis) using GraphPad Prism and fitted using a nonlinear regression model (four parameters). The results of the IC₅₀ of inhibition of nucleosome sliding are shown in FIG. 6. IC₅₀ values are presented by the following symbols: +++: IC₅₀ <25 μM; ++: IC₅₀=25-100 μM; +: IC₅₀=100-250 μM; and ‘-’: compound is not active, i.e. never observe inhibition >50%.

### SAR Analysis of ALC1 Inhibitors

In general, the ALCi cluster presented here has straightforward SAR clearly indicated by the presence of a large number of molecular match pairs. For example, in the R⁶ substitution, there is a clear preference for halogens over either oxygen or methyl substitutions, as demonstrated by the improved potency of ALCi-1002 over ALCi-1009 and ALCi-1055. Additionally, in the R⁴ region, there is a preference the tetrazole over the carboxylic acid (no tetrazole containing compound with IC50 >25 μM). In the R⁵ region of the molecule, nonaromatic, hydrophobic moieties tend to be preferred for increased potency and selectivity in cells, especially when combined with a tetrazole in the R⁴ region (ALCi-1013 vs ALCi-1002). By extrapolating from the currently available SAR, the skilled person should easily be able to develop additional ALC1 inhibitors in the same vein of those presented below.

### Synthesis of ALC1 Inhibitors

The synthesis of compounds of general formula (I) can be carried out according to the general synthesis scheme shown in FIGS. 9 and 10. The compounds prepared in FIGS. 9 and 10 reflect a basic compound scaffold in which the phenyl groups can be substituted at any position, not only where the R groups are depicted.

### LIST OF REFERENCES

Abbott, J. M., Zhou, Q., Esquer, H., Pike, L., Broneske, T. P., Rinaldetti, S., Abraham, A. D., Ramirez, D. A., Lunghofer, P. J., Pitts, T. M., Regan, D. P., Tan, A. C., Gustafson, D. L., Messersmith, W. A. & LaBarbera, D. V. (2020). First-in-Class Inhibitors of Oncogenic CHD1L with Preclinical Activity against Colorectal Cancer. Molecular Cancer Therapeutics, 19(8), 1598-1612. https://doi.org/10.1158/1535-7163.mct-20-0106.
Ahel, D., Horejsi, Z., Wiechens, N., Polo, S. E., Garcia-Wilson, E., Ahel, I., Flynn, H., Skehel, M., West, S. C., Jackson, S. P., et al. (2009). Poly(ADP-ribose)-dependent regulation of DNA repair by the chromatin remodeling enzyme ALC1. Science. 325, 1240-1243.Cheng, W., Su, Y., and Xu, F. (2013). CHD1L: a novel oncogene. Mol. Cancer 12, 170.
Bushweller J H. (2019) Targeting transcription factors in cancer—from undruggable to reality, NatuRe Reviews, 19: 611-624.
Charifson, P S.; Kuntz, I D. Recent successes and continuing limitations in computer aided drug design. In: Charifson, P S., editor. Practical Application of Computer Aided Drug Design. New York: Dekker; 1997.
Flaus A., Martin D M A, Barton G J, Owen-Hughes T (2006), Identification of multiple distinct Snf2 subfamilies with conserved structural motifs Nucleic Acids Res.; 34(10): 2887-2905.
Greer J, Erickson J W, Baldwin J J, Varney M D. Application of the three-dimensional structures of protein target molecules in structure-based drug design. J Med Chem 1994; 37:1035-1047.
Gottschalk, A. J., Timinszky, G., Kong, S. E., Jin, J., Cai, Y., Swanson, S. K., Washburn, M. P., Florens, L., Ladurner, A. G., Conaway, J. W., et al. (2009). Poly(ADP-ribosyl)ation directs recruitment and activation of an ATP-dependent chromatin remodeler. Proc. Natl. Acad. Sci. 106, 13770-13774.
Hazuda D J, Anthony N J, Gomez R P, Jolly S M, Wai J S, Zhuang L, Fisher T E, Embrey M, Guare J P Jr, Egbertson M S, et al. A nap-thyridine carboxamide provides evidence for discordant resistance between mechanistically identical inhibitors of HIV-1 integrase. Proc Natl Acad Sci USA 2004; 101:11233-11238
Human DNA repair genes. (n.d.). https://www.mdanderson.org/documents/Labs/Wood-Laboratory/human-dna-repair-genes.html
Jorgensen W L. The many roles of computation in drug discovery. Science 2004; 303:1813-1818.
Khan S, Yonghan He X, Zhang X, Yuan Y, Pu S, Kong Q, Zheng G, and Zhou D (2020) PROteolysis TArgeting Chimeras (PROTACs) as emerging anticancer therapeutics Oncogene (2020) 39:4909-4924.
Kim, K. B., Soroceanu, L., De Semir, D., Millis, S. Z., Ross, J. S., Vosoughi, E., Dar, A. A., Nosrati, M., Campisi, J., Ice, R. J., Chen, M. M., Chetal, K., Bhattacharjee, A., Moretto, J., Leong, S. P. L., Singer, M. I., Parrett, B. M., Minor, D. R., McAllister, S. D., . . . Kashani-Sabet, M. (2021). Prevalence of Homologous Recombination Pathway Gene Mutations in Melanoma: Rationale for a New Targeted Therapeutic Approach. Journal of Investigative Dermatology, 141(8), 2028-2036.e2. https://doi.org/10.1016/j.jid.2021.01.024
Lehmann, L. C., Hewitt, G., Aibara, S., Leitner, A., Marklund, E., Maslen, S. L., Maturi, V., Chen, Y., van der Spoel, D., Skehel, J. M., et al. (2017). Mechanistic insights into autoinhibition of the oncogenic chromatin remodeler ALC1. Mol. Cell 68, 847-859.
Li, Y., He, L. R., Gao, Y., Zhou, N. N., Liu, Y., Zhou, X. K., Liu, J. F., Guan, X. Y., Ma, N. F., and Xie, D. (2019). CHD1L contributes to cisplatin resistance by upregulating the ABCB1-NF-κB axis in human non-small-cell lung cancer. Cell Death Dis. 10, 1-17.
Liverton N J, Holloway M K, McCauley J A, Rudd M T, Butcher J W, Carroll S S, DiMuzio J, Fandozzi C, Gilbert K F, Mao S S, et al. Molecular modeling based approach to potent P2-P4 macrocyclic inhibitors.
Lord, C. J., and Ashworth, A. (2012). The DNA damage response and cancer therapy. Nature 481, 287-294.
Luijsterburg, M. S., de Krijger, I., Wiegant, W. W., Shah, R. G., Smeenk, G., de Groot, A. J. L., Pines, A., Vertegaal, A. C. O., Jacobs, J. J. L., Shah, G. M., et al. (2016). PARP1 links CHD2-mediated chromatin expansion and H_{3.3} deposition to DNA repair by non-homologous endjoining. Mol. Cell 61, 547-562.
Mehrotra, P. V., Ahel, D., Ryan, D. P., Weston, R., Wiechens, N., Kraehenbuehl, R., Owen-Hughes, T., and Ahel, I. (2011). DNA repair factor APLF is a histone chaperone. Mol. Cell 41, 46-55.
Murai, J., Huang, S.-N., Das, B. B., Renaud, A., Zhang, Y., Doroshow, J. H., Ji, J., Takeda, S. & Pommier, Y. (2012). Trapping of PARPI and PARP2 by Clinical PARP Inhibitors. Cancer Research, 72(21), 5588-5599. https://doi.org/10.1158/0008-5472.can-12-2753
Murai, J., Huang, S.-Y. N., Renaud, A., Zhang, Y., Ji, J., Takeda, S., Morris, J., Teicher, B., Doroshow, J. H., and Pommier, Y. (2014). Stereospecific PARP trapping by BMN 673 and comparison with olaparib and rucaparib. Mol. Cancer Ther. 13, 433-443
Peng, G., Lin, C. C. J., Mo, W., Dai, H., Park, Y., Kim, S., Peng, Y., Mo, Q., Siwko, S., Hu, R., Lee, J., Hennessy, B. T., Hanash, S. M., Mills, G. B., & Lin, S. Y. (2014). Genome-wide transcriptome profiling of homologous recombination DNA repair. Nature Communications, 5(1). https://doi.org/10.1038/ncomms4361
Ray Chaudhuri, A., and Nussenzweig, A. (2017). The multifaceted roles of PARP1 in DNA repair and chromatin remodelling. Nat. Rev. Mol. Cell Biol. 18, 610-621.
Sellou, H., Lebeaupin, T., Chapuis, C., Smith, R., Hegele, A., Singh, H. R., Kozlowski, M., Bultmann, S., Ladurner, A. G., Timinszky, G., et al. (2016). The poly(ADP-ribose)-dependent chromatin remodeler Alc1 induces local chromatin relaxation upon DNA damage. Mol. Biol. Cell 27, 3791-3799.
Singh, H. R., Nardozza, A. P., Möller, I. R., Knobloch, G., Kistemaker, H. A. V., Hassler, M., Harrer, N., Blessing, C., Eustermann, S., Kotthoff, C., et al. (2017a). A poly-ADP-ribose trigger releases the auto-inhibition of a chromatin remodeling oncogene. Mol. Cell 68, 860-871.
Smeenk, G., Wiegant, W. W., Marteijn, J. A., Luijsterburg, M. S., Sroczynski, N., Costelloe, T., Romeijn, R. J., Pastink, A., Mailand, N., Vermeulen, W., et al. (2013). Poly(ADP-ribosyl)ation links the chromatin remodeler SMARCA5/SNF2H to RNF168-dependent DNA damage signaling. J. Cell Sci. 126, 889-903.
Su, F. R., Ding, J. H., Bo, L., and Liu, X. G. (2014). Chromodomain helicase/ATPase DNA binding protein 1-like protein expression predicts poor prognosis in nasopharyngeal carcinoma. Exp. Ther. Med. 8, 1745-1750.
Timinszky, G., Till, S., Hassa, P. O., Hothorn, M., Kustatscher, G., Nijmeijer, B., Colombelli, J., Altmeyer, M., Stelzer, E. H. K., Scheffzek, K., et al. (2009). A macrodomain-containing histone rearranges chromatin upon sensing PARPI activation. Nat. Struct. Mol. Biol. 16, 923-929.
Toh, M., & Ngeow, J. (2021). Homologous Recombination Deficiency: Cancer Predispositions and Treatment Implications. Oncologist, 26(9), e1526-e1537. https://doi.org/10.1002/onco.13829
U-2 OS ATCC® HTB-96™ Homo sapiens bone osteosarcoma. (2016). ATCC.https://www.lgcstandars-atcc.org/products/all/HTB-96.aspx?geo_country=de#characteristics
Schames J R, Henchman R H, Siegel J S, Sotriffer C A, Ni H, McCammon J A. Discovery of a novel binding trench in HIV integrase. J Med Chem 2004; 47:1879-1881.Salomon-Ferrer, R., Case, D. A. and Walker, R. C. (2013) ‘An Overview of the Amber Biomolecular Simulation Package’, WIREs Comput. Mol. Sci., 3, pp. 198-210.
Yamamoto, H., & Hirasawa, A. (2021). Homologous Recombination Deficiencies and Hereditary Tumors. International Journal of Molecular Sciences, 23(1), 348. https://doi.org/10.3390/ijms23010348
Zandarashvili, L., Langelier, M.-F., Velagapudi, U. K., Hancock, M. A., Steffen, J. D., Billur, R., Hannan, Z. M., Wicks, A. J., Krastev, D. B., Pettitt, S. J., Lord, C. J., Talele, T. T., Pascal, J. M. & Black, B. E. (2020). Structural basis for allosteric PARP-1 retention on DNA breaks. Science, 368(6486), eaax6367. https://doi.org/10.1126/science.aax6367
Karras, G. I., Kustatscher, G., Buhecha, H. R., Allen, M. D., Pugieux, C., Sait, F., Bycroft, M. & Ladurner, A. G. (2005). The macro domain is an ADP-ribose binding module. The EMBO Journal, 24(11), 1911-1920. https://doi.org/10.1038/sj.emboj.7600664
Zimmermann, M., Murina, O., Reijns, M. A. M., Agathanggelou, A., Challis, R., Tarnauskaite, Z. 6., Muir, M., Fluteau, A., Aregger, M., McEwan, A., Yuan, W., Clarke, M., Lambros, M. B., Paneesha, S., Moss, P., Chandrashekhar, M., Angers, S., Moffat, J., Brunton, V. G., . . . Durocher, D. (2018). CRISPR screens identify genomic ribonucleotides as a source of PARP-trapping lesions. Nature, 559(7713), 285-289. https://doi.org/10.1038/s41586-018-0291-z
von Itzstein M, Wu W Y, Kok G B, Pegg M S, Dyason J C, Jin B, Van Phan T, Smythe M L, White H F, Oliver S W, et al. Rational design of potent sialidase-based inhibitors of influenza virus replication. Nature 1993; 363:418-423.

1. A method of treating or ameliorating a proliferative disease in a patient in need thereof comprising administering an inhibitor of ALC1 (ALC1i) according to formula (I) 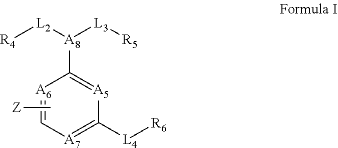
wherein
A5 and A8 are each independently selected from N or CH;
A6 is selected from N or CH, or when A6 takes part in the annulated carbo- or heterocycle Z, then A6 is C;
A7 is selected from N or CH, or when A7 takes part in the annulated carbo- or heterocycle Z, then A7 is C;
L2 is selected from the group consisting of —CH₂—R4, —CF₂—R4, —CH₂—CH₂—R4, —CH₂—CH₂—CH₂—R4, —O—R4, —NH—R4, —N═R4;
L3 is selected from the group consisting of CH₂—R5, —CF₂—R5, —CH₂—CH₂—R5, —CH₂—CF₂—R5, —CH₂—CH₂—CH₂—R5, —O—R5, —NH—R5, —N═R5;
or
L2 and L3 together with the A8 to which they are connected form a 5- or 6-membered heterocycle substituted by R⁴ and/or R⁵;
L4 is CH₂, —CF₂—, CH₂—CH₂, CH₂—CH₂—CH₂, 0, N, and NH, or is absent;
Z is a 5-, 6- or 7-membered carbo- or heterocycle, optionally substituted with one, two, or three, preferably one substituent(s) selected from the group consisting of —Br, —Cl, —F, —I, —OH, Me, —CF₃, Et, —OMe, —SMe, and —NO₂;
and can be annulated to the central core or connected via a covalent bond
R⁴ is 5-, 6- or 7-membered carbo- or heterocycle, optionally substituted with one, two, or three, preferably one substituent(s) selected from the group consisting of —Br, —Cl, —F, —I, —CF₃, Me, Et, —OMe, and —SMe, or R⁴ is hydrogen, methyl, or COOH;
R⁵ is a 4-, 5-, 6-, 7-, 8-, 9- or 10-membered carbo- or heterocycle, optionally substituted with one, two, or three (preferably one) substituent(s) selected from the group consisting of —Br, —Cl, —F, —I, Me, —CF₃, Et, —OMe, and —SMe;
R⁶ is a 5-, 6- or 7-membered carbo- or heterocycle, optionally substituted with one, two, or three (preferably one) substituent(s) selected from the group consisting of —Br, —Cl, —F, —I, —OH, —NO₂, Me, —CF₃, Et, —OMe, and —SMe;
or R⁶ is H;
or
when A7 takes part in the annulated carbo- or heterocycle Z then A5 and A6 are independently selected from —N or —CH and A8 is selected from —N, —CH, —CH₂—N, —CH₂—CH, or —NH—CH;
or pharmaceutically acceptable salt thereof.

2. The method according to claim 1, wherein
A5 is N;
one of A6 and A7 is CH and the other one is N; or one of A6 and A7 is C and takes part in the annulated carbo- or heterocycle Z and the other one is N;
A8 is N or CH,
or
A5 is N;
one of A6 and A7 is C and takes part in the annulated 5-, 6- or 7-membered carbo- or heterocycle, preferably 5- or 6 membered aryl or heteroaryl Z and the other one is N;
A8 is N;
L2 is CH₂—CH₂—R4 and L3 is CH₂—CH₂—R5 or CH₂—CF₂—R5 or L2 and L3 together with the A8 to which they are connected form a 5- or 6-membered heterocycle, preferably piperidinyl or a pyrrolidinyl substituted by R4 and R5;
L4 is absent;
Z is any 6-membered carbo- or heterocycle, preferably 6-membered aryl or heteroaryl annulated to the central core, wherein Z is optionally substituted with one, two, or three, preferably one substituent(s) selected from the group consisting of —Br, —Cl, —F, —I, —OH, Me, —CF₃, Et, —OMe, —SMe, and NO₂;
R4 is COOH or tetrazolyl;
R5 is any 4-, 5-, 6-, or 7-membered carbo- or heterocycle, optionally substituted with one, two, or three, preferably one substituent(s) selected from the group consisting of —Br, —Cl, —F, —I, Me, —CF₃, Et, —OMe, and —SMe;
R6 is any 5- or 6-membered carbo- or heterocycle, optionally substituted with one, two, or three (preferably one) substituents selected from the group consisting of —Br, —Cl, —F, —I, —OH, —NO₂, Me, —CF₃, Et, —OMe, and —SMe, or R6 is H.

3. The method according to claim 1, wherein
each one of A5, A7 and A8 is N;
A6 is C and takes part in the annulated carbo- or heterocycle Z;
L3 is CH₂—CF₂—R5 or each one of L2 and L3 is CH₂—CH₂; or L2 and L3 together with the A8 to which they are connected form a piperidine ring or a pyrrolidine ring, substituted by R4 and R5;
L4 is absent;
Z is phenyl or cyclohexyl annulated to the central core, wherein Z is optionally substituted with one, two, or three, preferably one substituent(s) selected from the group consisting of —Br, —Cl, —F, —OH, Me, —CF₃, —OMe, and —NO₂,
R4 is COOH or tetrazolyl;
R5 is phenyl, cyclobutyl, cyclopentyl or adamantyl, optionally substituted with one, two, or three, preferably one substituent(s) selected from the group consisting of —Br, —Cl, —CF₃, Me, —CH₂—CF₃, and —OMe;
R6 is any 6-membered carbo- or heterocycle (preferably phenyl or cyclohexyl; more preferably phenyl), optionally substituted with one, two, or three, preferably one substituent(s) selected from the group consisting of Br, —Cl, —F, Me, —CF₃, —OMe, and —NO₂.

4. The method according to claim 1, wherein the ALC1 is selected from the group consisting of:

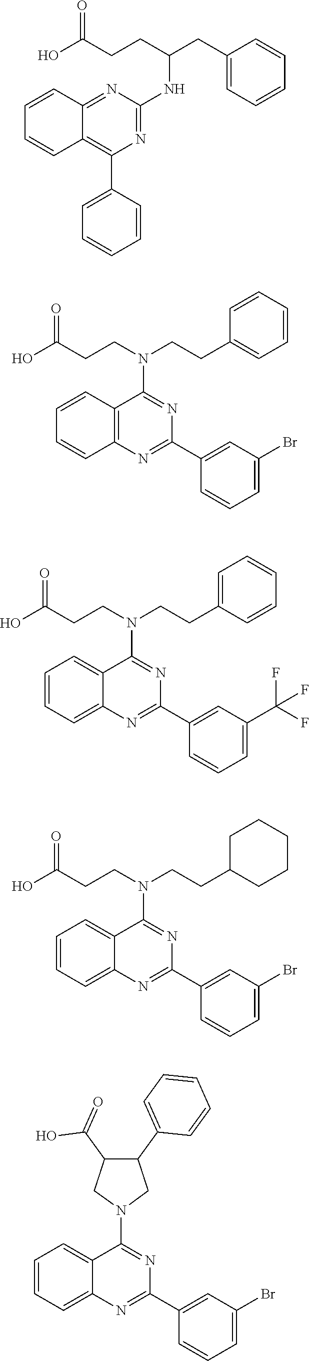 [SMILES: O=C(O)CCC(Cc1ccccc1)Nc1nc(-c2ccccc2)c2ccccc2n1 ; O=C(O)CCN(CCc1ccccc1)c1nc(-c2cccc(Br)c2)nc2ccccc12 ; O=C(O)CCN(CCc1ccccc1)c1nc(-c2cccc(C(F)(F)F)c2)nc2ccccc12 ; O=C(O)CCN(CCC1CCCCC1)c1nc(-c2cccc(Br)c2)nc2ccccc12 ; O=C(O)C1CN(c2nc(-c3cccc(Br)c3)nc3ccccc23)CC1c1ccccc1]

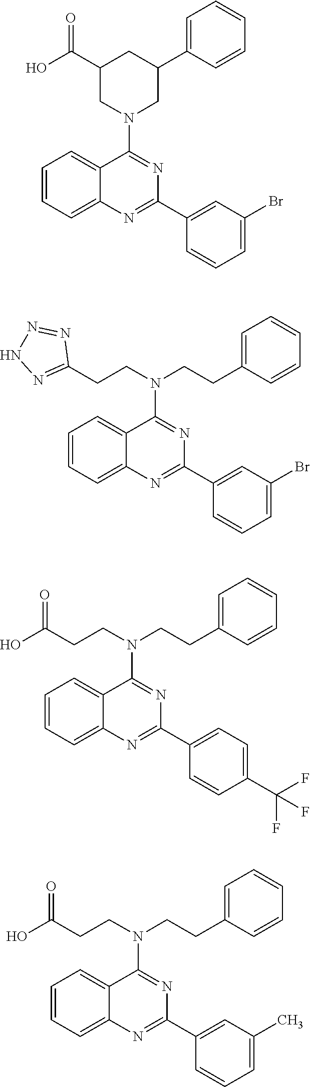

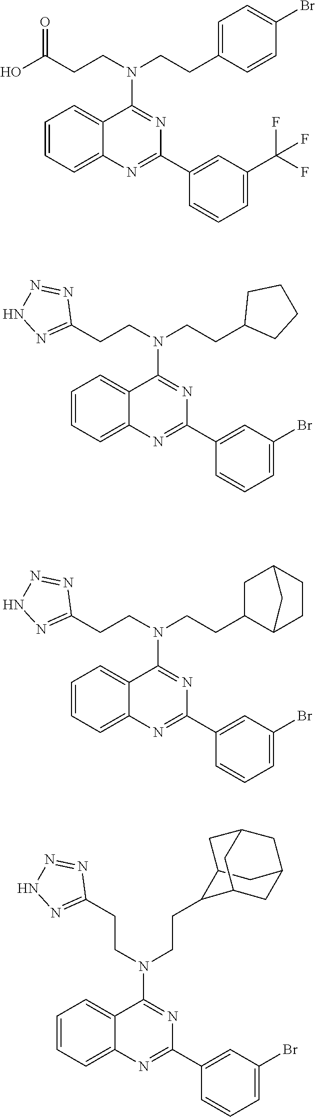 [SMILES: O=C(O)CCN(CCc1ccc(Br)cc1)c1nc(-c2cccc(C(F)(F)F)c2)nc2ccccc12 ; Brc1cccc(-c2nc(N(CCc3nn[nH]n3)CCC3CCCC3)c3ccccc3n2)c1 ; Brc1cccc(-c2nc(N(CCc3nn[nH]n3)CCC3CC4CCC3C4)c3ccccc3n2)c1 ; Brc1cccc(-c2nc(N(CCc3nn[nH]n3)CCC3C4CC5CC(C4)CC3C5)c3ccccc3n2)c1]

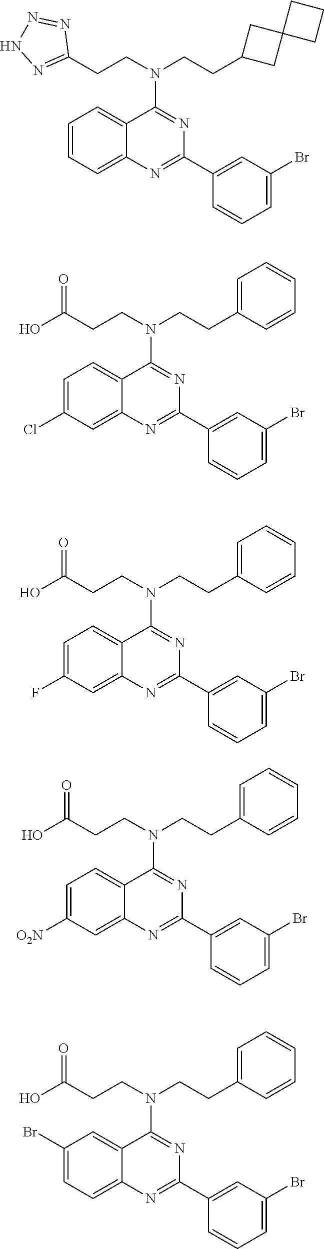

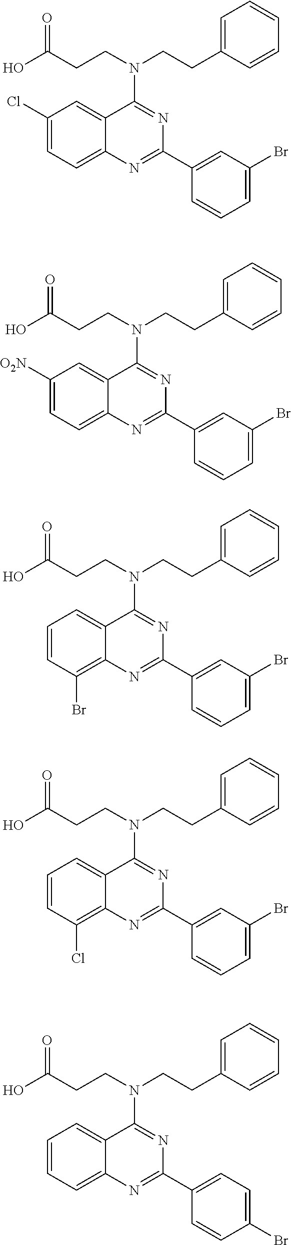

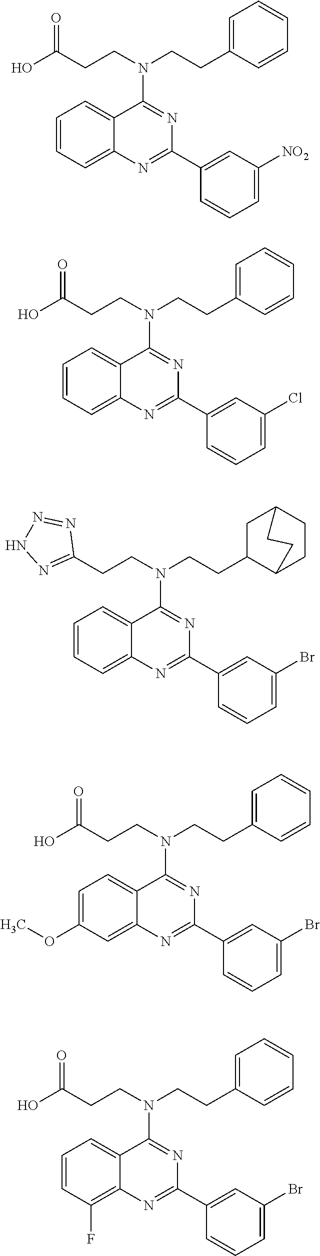

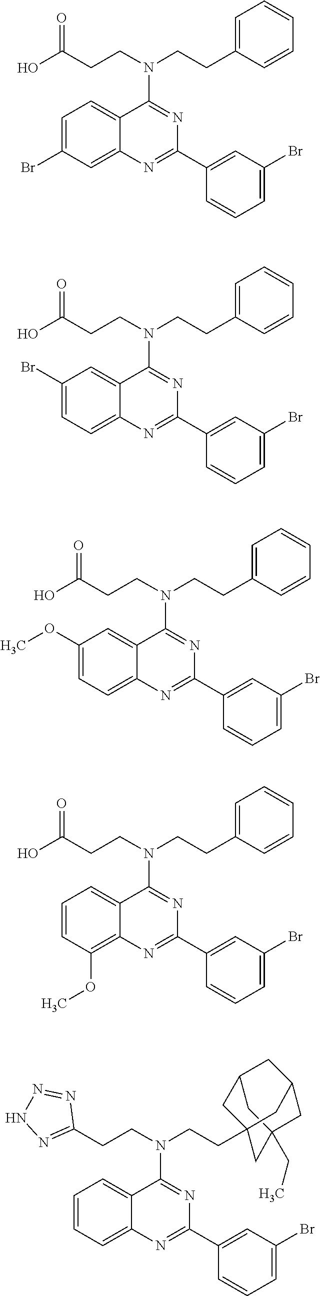

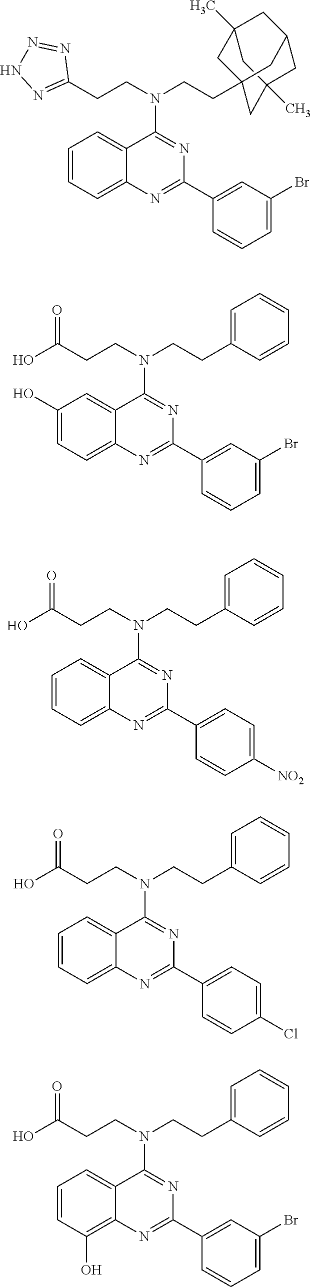

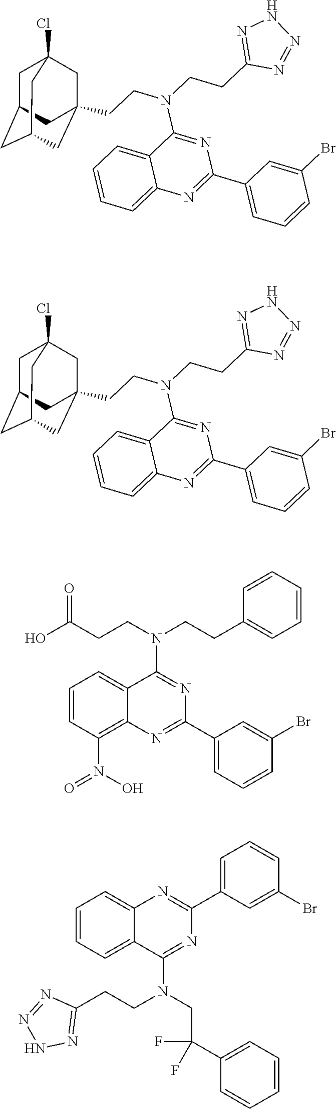

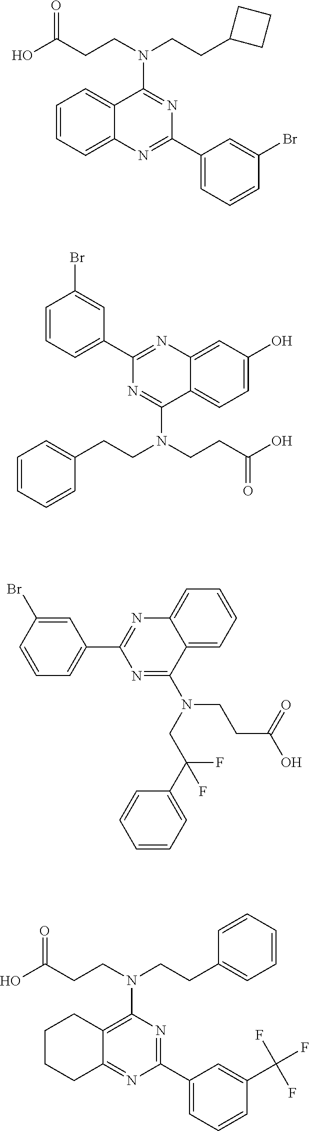 [SMILES: O=C(O)CCN(CCC1CCC1)c1nc(-c2cccc(Br)c2)nc2ccccc12 ; O=C(O)CCN(CCc1ccccc1)c1nc(-c2cccc(Br)c2)nc2cc(O)ccc12 ; O=C(O)CCN(CC(F)(F)c1ccccc1)c1nc(-c2cccc(Br)c2)nc2ccccc12 ; O=C(O)CCN(CCc1ccccc1)c1nc(-c2cccc(C(F)(F)F)c2)nc2c1CCCC2]

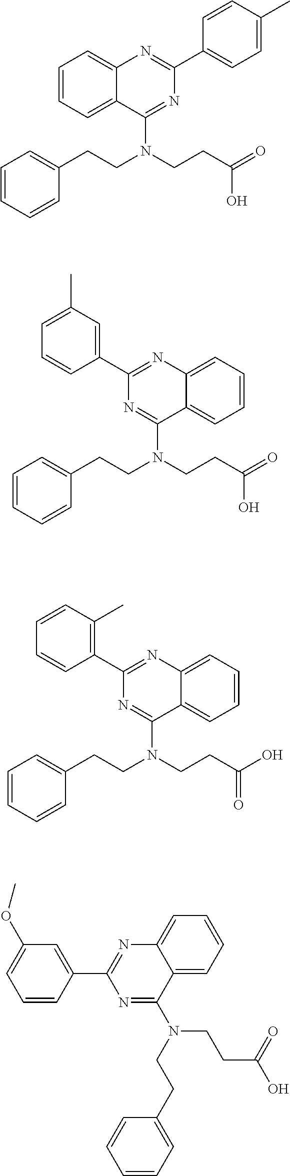 [SMILES: Cc1ccc(-c2nc(N(CCC(=O)O)CCc3ccccc3)c3ccccc3n2)cc1 ; Cc1cccc(-c2nc(N(CCC(=O)O)CCc3ccccc3)c3ccccc3n2)c1 ; Cc1ccccc1-c1nc(N(CCC(=O)O)CCc2ccccc2)c2ccccc2n1 ; COc1cccc(-c2nc(N(CCC(=O)O)CCc3ccccc3)c3ccccc3n2)c1]

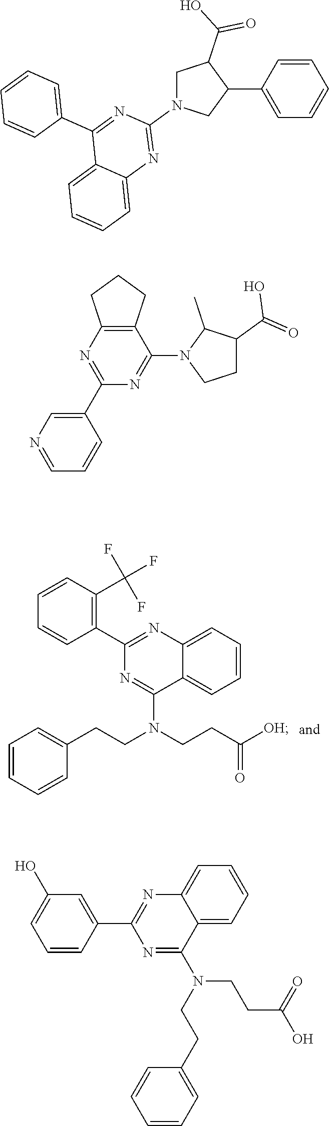 [SMILES: O=C(O)C1CN(c2nc(-c3ccccc3)c3ccccc3n2)CC1c1ccccc1 ; CC1C(C(=O)O)CCN1c1nc(-c2cccnc2)nc2c1CCC2 ; O=C(O)CCN(CCc1ccccc1)c1nc(-c2ccccc2C(F)(F)F)nc2ccccc12 ; O=C(O)CCN(CCc1ccccc1)c1nc(-c2cccc(O)c2)nc2ccccc12]

5. The method of treating or ameliorating a proliferative disease in a patient in need thereof comprising administering a bifunctional compound comprising the ALC1i according to claim 1 and a compound which recruits E3 ubiquitin ligase to ALC1 (E3 recruiter), wherein the ALC1i and the E3 recruiter are covalently linked.

6. The method of treating a proliferative disease in a patient in need thereof comprising administering a pharmaceutical composition comprising the ALC1i according to claim 1 or the bifunctional compound of claim 5 and a pharmaceutically acceptable excipient.

7. The method according to claim 1, wherein the treating and ameliorating the proliferative disease comprises the administration of said ALC1i and the administration of a Poly(ADP-ribose)-Polymerase inhibitor (PARPi).

8. (canceled)

9. The method according to claim 7, wherein the PARPi lowers PARP activity and/or inhibits PARP1, PARP2 and/or PARP3, wherein preferably the PARPi
(i) that lowers PARP activity is selected from small interfering RNA, and
(ii) that inhibits PARP1 is selected from the group consisting of a compound of
(a) formula (II) 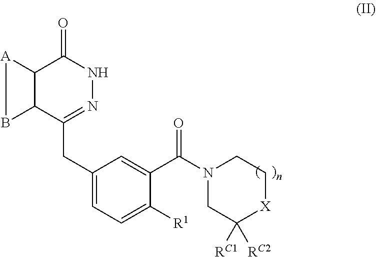
and isomers, salts, solvates, chemically protected forms, and prodrugs thereof wherein:
A and B together represent an optionally substituted, fused aromatic ring;
X is NR^{X} or CR^{X}R^{Y};
if X═NR^{X}then n is 1 or 2 and if X═CR^{X}R^{Y} then n is 1;
R^{X} is selected from the group consisting of H, optionally substituted C₁₋₂₀ alkyl, C₅₋₂₀ aryl, C₃₋₂₀ heterocyclyl, amido, thioamido, ester, acyl, and sulfonyl groups;
R^{Y} is selected from H, hydroxy, amino;
or R^{X} and R^{Y} may together form a spiro-C₃₋₇ cycloalkyl or heterocyclyl group;
R^{C1} and R^{C2} are independently selected from the group consisting of hydrogen and C₁₋₄ alkyl or when X is CR^{X}R^{Y}, R^{C1}, R^{C2}, R^{X} and R^{Y}, together with the carbon atoms to which they are attached, may form an optionally substituted fused aromatic ring; and
R¹ is selected from H and halo;
and
(b) formula (III) 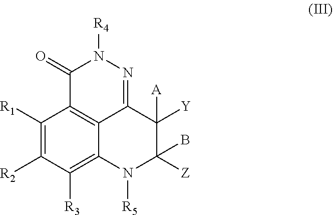
and isomers, salts, solvates, chemically protected forms, and prodrugs thereof wherein:
Y and Z are each independently selected from the group consisting of
1. an aryl group optionally substituted with 1, 2, or 3 R₆;
2. a heteroaryl group optionally substituted with 1, 2, or 3 R₆;
3. a substituent independently selected from the group consisting of hydrogen, alkenyl, alkoxy, alkoxyalkyl, alkoxycarbonyl, alkoxycarbonylalkyl, alkyl, alkynyl, arylalkyl, cycloalkyl, cycloalkylalkyl, haloalkyl, hydroxyalkylene, oxo, heterocycloalkyl, heterocycloalkylalkyl, alkylcarbonyl, arylcarbonyl, heteroarylcarbonyl alkylsulfonyl, arylsulfonyl, heteroarylsulfonyl, (R_{A}R_{B})alkylene, (NR_{A}R_{B})carbonyl, (NR_{A}R_{B})carbonylalkylene, (NR_{A}R_{B})sulfonyl, and (R_{A}R_{B})sulfonylalkylene;
wherein each R₆ is selected from OH, NO₂, CN, Br, Cl, F, I, C₁₋₆-alkyl, C₃₋₈-cycloalkyl, C₂₋₈-heterocycloalkyl; C₂₋₆-alkenyl, alkoxy, alkoxyalkyl, alkoxycarbonyl, alkoxycarbonylalkyl, C₂₋₆-alkynyl, aryl, arylalkyl, C₃₋₈-cycloalkylalkyl, haloalkoxy, haloalkyl, hydroxyalkylene, oxo, heteroaryl, heteroarylalkoxy, heteroaryloxy, heteroarylthio, heteroarylalkylthio, heterocycloalkoxy, C₂₋₈-heterocycloalkylthio, heterocyclooxy, heterocyclothio, NR_{A}R_{B}, (R_{A}R_{B})C₁₋₆-alkylene, (NR_{A}R_{B})carbonyl, (R_{A}R_{B})carbonylalkylene, (NR_{A}R_{B})sulfonyl, and (NR_{A}R_{B})sulfonylalkylene;
R₁, R₂, and R₃ are each independently selected from the group consisting of hydrogen, halogen, alkenyl, alkoxy, alkoxycarbonyl, alkyl, cycloalkyl, alkynyl, cyano, haloalkoxy, haloalkyl, hydroxyl, hydroxyalkylene, nitro, NR_{A}R_{B}, NR_{A}R_{B} alkylene, and (R_{A}R_{B})carbonyl;
A and B are each independently selected from hydrogen, Br, Cl, F, I, OH, C₁₋₆-alkyl, C₃₋₈-cycloalkyl, alkoxy, alkoxyalkyl wherein C₁₋₆-alkyl, C₃₋₈-cycloalkyl, alkoxy, alkoxyalkyl are optionally substituted with at least one substituent selected from OH, NO₂, CN, Br, Cl, F, I, C₁₋₆-alkyl, and C₃₋₈-cycloalkyl, wherein B is not OH;
R_{A}, and R_{B} are independently selected from the group consisting of hydrogen, alkyl, cycloalkyl, and alkylcarbonyl; or R_{A} and R_{B} taken together with the atom to which they are attached form a 3-10 membered heterocycle ring optionally having one to three heteroatoms or hetero functionalities selected from the group consisting of —O—, —NH, —N(C₁₋₆-alkyl)-, —NCO(C₁₋₆-alkyl)-, —N(aryl)-, —N(aryl-C₁₋₆-alkyl-), —N(substituted-aryl-C₁₋₆-alkyl-)-, —N(heteroaryl)-, —N(heteroaryl-C₁-C₆-alkyl-)-, —N(substituted-heteroaryl-C₁₋₆ alkyl-)-, and —S— or S(O)q-, wherein q is 1 or 2 and the 3-10 membered heterocycle ring is optionally substituted with one or more substituents;
R₄ and R₅ are each independently selected from the group consisting of hydrogen, alkyl, cycloalkyl, alkoxyalkyl, haloalkyl, hydroxyalkylene, and (NR_{A}R_{B})alkylene;
and isomers, salts, solvates, chemically protected forms, and prodrugs thereof;
(iii) that inhibits PARP1 and PARP2 is selected from the group consisting of a compound of
(a) formula (IV) 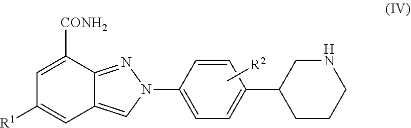
and isomers, salts, solvates, chemically protected forms, and prodrugs thereof wherein:
R₁ is hydrogen or fluorine; and
R₂ is hydrogen or fluorine;
and
(b) formula (V) 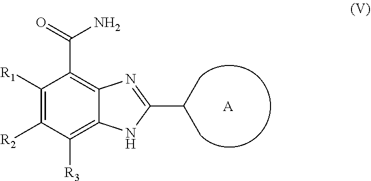
and isomers, salts, solvates, chemically protected forms, and prodrugs thereof wherein:
R₁, R₂, and R₃ are independently selected from the group consisting of hydrogen, alkenyl, alkoxy, alkoxycarbonyl, alkyl, alkynyl, cyano, haloalkoxy, haloalkyl, halogen, hydroxy, hydroxyalkyl, nitro, NR_{A}R_{B}, and (NR_{A}R_{B})carbonyl;
A is a nonaromatic 4, 5, 6, 7, or 8-membered ring that contains 1 or 2 nitrogen atoms and, optionally, one sulfur or oxygen atom, wherein the nonaromatic ring is optionally substituted with 1, 2, or 3 substituents selected from the group consisting of alkenyl, alkoxy, alkoxyalkyl, alkoxycarbonyl, alkoxycarbonylalkyl, alkyl, alkynyl, aryl, arylalkyl, cycloalkyl, cycloalkylalkyl, cyano, haloalkoxy, haloalkyl, halogen, heterocycle, heterocyclealkyl, heteroaryl, heteroarylalkyl, hydroxy, hydroxyalkyl, nitro, NR_{C}R_{D}, (NR_{C}R_{D})alkyl, (NR_{C}R_{D})carbonyl, (NR_{C}R_{D})carbonylalkyl, and (NR_{C}R_{D})sulfonyl; and
R_{A}, R_{B}, R_{C}, and R_{D} are independently selected from the group consisting of hydrogen, alkyl, and alkycarbonyl
(iii) that inhibits PARP1, PARP2 and PARP3 is a compound of formula (VI) 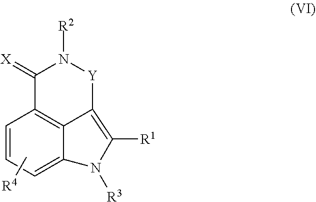
and isomers, salts, solvates, chemically protected forms, and prodrugs thereof wherein:
R₁ is: H; halogen; cyano; an optionally substituted alkyl, alkenyl, alkynyl, cycloalkyl, heterocycloalkyl, aryl, or heteroaryl group (e.g., unsubstituted or substituted with one or more substituents selected from halogen, hydroxy, nitro, and amino, alkoxy, alkyl, and aryl groups unsubstituted or substituted with one or more substituents selected from halogen, hydroxy, nitro, carboxy, and optionally substituted amino and ether groups (such as O-aryl)); or —C(O)—R₁₀, where R₁₀ is: H; an optionally substituted alkyl, alkenyl, alkynyl, cycloalkyl, heterocycloalkyl, aryl, or heteroaryl group (e.g., unsubstituted or substituted with one or more substituents selected from halogen, hydroxy, nitro, amino, and alkyl and aryl groups unsubstituted or substituted with one or more substituents selected from halo, hydroxy, nitro, and amino); or OR₁₀₀ or NR₁₀₀R₁₁₀, where R₁₀₀ and R₁₁₀ are each independently H or an optionally substituted alkyl, alkenyl, alkynyl, cycloalkyl, heterocycloalkyl, aryl, or heteroaryl group (e.g., unsubstituted or substituted with one or more substituents selected from alkyl, alkenyl, alkynyl, cycloalkyl, heterocycloalkyl, aryl, and heteroaryl groups unsubstituted or substituted with one or more substituents selected from halogen, hydroxy, nitro, amino, and alkyl and aryl groups unsubstituted or substituted with one or more substituents selected from halogen, hydroxy, nitro, and optionally substituted amino groups);
R₂ is H or alkyl;
R₃ is H or alkyl;
R₄ is H, halogen or alkyl;
X is O or S;
Y is (CR₅R₆)(CR₇R₈), or N—C(R₅), where:
n is 0 or 1;
R₅ and R₆ are each independently H or an optionally substituted alkyl, alkenyl, alkynyl, cycloalkyl, heterocycloalkyl, aryl, or heteroaryl group (e.g., unsubstituted or substituted with one or more substituents selected from halogen, hydroxy, nitro, amino, and lower alkyl, lower alkoxy, or aryl groups unsubstituted or substituted with one or more substituents selected from halogen, hydroxy, nitro, and amino); and
R₇ and R₈ are each independently H or an optionally substituted alkyl, alkenyl, alkynyl, cycloalkyl, heterocycloalkyl, aryl, or heteroaryl group (e.g., unsubstituted or substituted with one or more substituents selected from halogen, hydroxy, nitro, amino, and lower alkyl, lower alkoxy, and aryl groups unsubstituted or substituted with one or more substituents selected from halogen, hydroxy, nitro, and amino);
where when R₁, R₄, R₅, R₆, and R₇ are each H, R₈ is not unsubstituted phenyl.

10. The method according to claim 7, wherein the PARPi is selected from the group consisting of Olaparib, Talazoparib, Niraparib, Rucaparib, and Veliparib, in particular of Veliparib, Olaparib, and Talazoparib.

11. The method according to claim 7, wherein the proliferative disease is selected from a BRCA-1 and/or BRCA-2-deficient tumor, and a tumor in which expression of PARP-1, PARP-2, PARP-3 and/or ALC1 is increased in comparison to non-tumor cells, and/or wherein the proliferative disease is selected from hepatocellular carcinoma, breast cancer, ovarian cancer, prostate cancer, colorectal cancer.

12. The method according to claim 7, wherein the PARPi and ALC1i or bifunctional compound are administered concomitantly or separately.

13. A kit of parts comprising separately packaged a PARPi and an ALC1i as specified in claim 1 or a composition comprising a PARPi and an ALC1i as specified in claim 1, and instructions for use to treat or ameliorate a proliferative disease.

14. The method according to claim 1, wherein the proliferative disease is selected from a BRCA1 and/or 2-deficient tumor, and a tumor in which expression of PARP1, PARP2, PARP3 and/or ALC1 is increased in comparison to non-tumor cells.

15. A method of treating or ameliorating a proliferative disease in a patient in need thereof comprising administering a bifunctional compound comprising the ALC1i according to claim 1 and a compound which recruits E3 ubiquitin ligase to ALC1 (E3 recruiter), wherein the ALC1i and the E3 recruiter are covalently linked through a linker.

16. A method of treating a proliferative disease in a patient in need thereof comprising administering a pharmaceutical composition comprising the bifunctional compound of claim 5 and a pharmaceutically acceptable excipient.

17. A method of treating a proliferative disease in a patient in need thereof comprising administering a pharmaceutical composition comprising the bifunctional compound of claim 15 and a pharmaceutically acceptable excipient.

18. The method according to claim 9, wherein the PARPi lowers PARP2 activity and/or inhibits PARP2 on chromatin.

19. A kit of parts comprising separately packaged a PARPi and a bifunctional compound according to claim 5 or a composition comprising a PARPi and a bifunctional compound according to claim 5, and instructions for use to treat or ameliorate a proliferative disease.
